(19) United States
(12) Patent Application Publication
(10) Pub. No.: US 2025/0257341 A1
(43) Pub. Date: Aug. 14, 2025

(54) COMPOSITIONS AND METHODS FOR EPIGENOME EDITING

(71) Applicant: Duke University, Durham, NC (US)

(72) Inventors: Charles A. Gersbach, Chapel Hill, NC (US); Isaac Hilton, Houston, TX (US)

(21) Appl. No.: 18/952,981

(22) Filed: Nov. 19, 2024

Related U.S. Application Data
(63) Continuation of application No. 17/471,935, filed on Sep. 10, 2021, now Pat. No. 12,215,366.
Application No. 17/471,935 is a division of application No. 16/865,151, filed on May 1, 2020, now Pat. No. 11,155,796.
Application No. 16/865,151 is a division of application No. 15/549,842, filed on Aug. 9, 2017, now Pat. No. 10,676,726.
(60) Provisional application No. 62/113,569, filed on Feb. 9, 2015.

(51) Int. Cl. C12N 9/22; C12N 9/10; C12N 15/11; C12N 15/63; C12N 15/85

(52) U.S. Cl. CPC C12N 9/22; C12N 9/1029; C12N 15/11; C12N 15/63; C12N 15/85; C12Y 203/01048; C07K 2319/80; C12N 2310/20

(57) ABSTRACT

Disclosed herein are CRISPR/Cas9-based gene activation systems that include a fusion protein of a Cas9 protein and a protein having histone acetyltransferase activity, and methods of using said systems.

### CROSS REFERENCE TO RELATED APPLICATIONS

This patent application is a continuation of Ser. No. 17/471,935, filed Sep. 10, 2021, which is a divisional of Ser. No. 16/865,151, filed May 1, 2020, which is a divisional of U.S. patent application Ser. No. 15/549,842, filed Aug. 9, 2017, which is the U.S. national stage entry, under 35 U.S.C. § 371, of International Application Number PCT/US2016/017221, filed Feb. 9, 2016, which claims priority to U.S. Provisional Application No. 62/113,569, filed Feb. 9, 2015, the entire contents of each of which are incorporated herein by reference.

### STATEMENT OF GOVERNMENT INTEREST

This invention was made with Government support under Federal Grant No. 1R01DA036865 awarded by the National Institutes of Health. The Government has certain rights to this invention.

### SEQUENCE LISTING

The sequence listing is filed with the application in electronic format only and is incorporated by reference herein. The sequence listing XML file named “028193-9190-US04_Sequence Listing” was created on Apr. 4, 2025, and is 407,760 bytes in size.

### TECHNICAL FIELD

The present disclosure is directed to CRISPR/Cas9-based gene activation systems and methods of using said systems.

### BACKGROUND

The Human Genome Project was funded and pursued based on the premise that the sequencing of the human genome would reveal the genetic basis for complex diseases that have a strong inheritable component, including cardiovascular disease, neurodegenerative conditions, and metabolic diseases such as diabetes. It was believed that this information would lead to new drug targets for these widespread diseases. However, thousands of genome-wide association studies (GWAS) have shown that the genetic variation associated with these complex diseases does not occur within genes, but rather in intergenic regulatory regions that control the levels of particular genes. Similarly, approximately 20% of Mendelian disorders do not have a detectable coding mutation, suggesting that the causal mutation is in a gene regulatory element. Importantly, it is very difficult to assign functional roles to these regulatory elements as they often are located in distant locations from their target genes. Moreover, many genes and regulatory elements fall into each positive hit of each GWAS study. In fact, follow-up projects to the Human Genome Project, such as the NIH-funded Encyclopedia of DNA Elements (ENCODE) and the Roadmap Epigenomics Project, have identified millions of putative regulatory elements across the human genome for many human cell types and tissues.

A primary challenge of functional genomics is to develop technologies that directly and precisely manipulate genome function at individual loci. Projects such as ENCODE and the Roadmap Epigenomics Project have identified millions of epigenetic marks across the human genome for many human cell types and tissues. Studying the function of those marks, however, has been largely limited to statistical associations with gene expression. Technologies for targeted direct manipulation of these epigenetic properties are necessary to transform such association-based findings into mechanistic principles of gene regulation. Such advances have the potential to benefit human health, as they could lead to gene therapies that modify the epigenetic code at targeted regions of the genome, strategies for regenerative medicine and disease modeling based on the epigenetic reprogramming of cell lineage specification, and the engineering of epigenome-specific drug screening platforms.

Manipulation of the epigenome is possible by treating cells with small molecule drugs, such as inhibitors of histone deacetylases or DNA methyltransferases, or differentiating cells into specific lineages. However, small molecule-based methods globally alter the epigenome and transcriptome, and are not suitable for targeting individual loci. Epigenome editing technologies, including the fusion of epigenome-modifying enzymes to programmable DNA-binding proteins such as zinc finger proteins and transcription activator-like effectors (TALEs), have been effective at achieving targeted DNA methylation, DNA hydroxymethylation, and histone demethylation, methylation, and deacetylation.

Fused to activation domains, such as oligomers of the herpes simplex viral protein 16 (VP16), dCas9 can function as a synthetic transcriptional regulator. However, limitations in the use of dCas9 activators remain, including the need for multiple activation domains or combinations of gRNAs to achieve high levels of gene induction by synergistic effects between activation domains. The conventional activator domains used in these engineered transcriptional factors, such as the VP16 tetramer VP64, function as a scaffold for recruiting multiple components of the preinitiation complex and do not have direct enzymatic function to specifically modulate the chromatin state. This indirect method of epigenetic remodeling does not allow for testing the role of specific epigenetic marks and may not be as potent as the direct programming of epigenetic states. There remains a need for the ability to target direct manipulation of epigenetic properties.

### SUMMARY

The present invention is directed to a fusion protein comprising two heterologous polypeptide domains, wherein the first polypeptide domain comprises a Clustered Regularly Interspaced Short Palindromic Repeats associated (Cas) protein and the second polypeptide domain

The present invention is directed to a DNA targeting system comprising the fusion protein, described above, and at least one guide RNA (gRNA).

The present invention is directed to a method of activating gene expression of a target gene in a cell, the method comprising contacting the cell with a polynucleotide encoding a DNA targeting system, wherein the DNA targeting system comprises the fusion protein, described above, and at least one guide RNA (gRNA).

### BRIEF DESCRIPTION OF THE DRAWINGS

FIGS. 1A-1C show that dCas9^{p300 Core} fusion protein activates transcription of endogenous genes from proximal promoter regions. FIG. 1A shows a schematic of dCas9 fusion proteins dCas9^{VP64} dCas9^{FL p300}, and dCas9^{p300 Core} Streptococcus pyogenes dCas9 contains nuclease inactivating mutations D10A and H840A. The D1399 catalytic residue in the p300 HAT domain is indicated. FIG. 1B shows Western blot showing expression levels of dCas9 fusion proteins and GAPDH in co-transfected cells (full blot shown in FIG. 7C). FIG. 1C shows relative mRNA expression of IL1RN, MYOD, and OCT4, determined by qRT-PCR, by the indicated dCas9 fusion protein co-transfected with four gRNAs targeted to each promoter region (Tukey-test, *P-value <0.05, n=3 independent experiments each, error bars: s.e.m.). Numbers above bars indicate mean expression. FLAG, epitope tag; NLS, nuclear localization signal; HA, hemagglutinin epitope tag; CH, cysteine-histidine-rich region; Bd, bromodomain; HAT, histone acetyltransferase domain.

FIGS. 2A-2C show that dCas9^{p300 Core} fusion protein activates transcription of endogenous genes from distal enhancer regions. FIG. 2A shows relative MYOD mRNA production in cells co-transfected with a pool of gRNAs targeted to either the proximal or distal regulatory regions and dCas9^{VP64} or dCas9^{p300 Core}; promoter data from FIG. 1C (Tukey-test, *P-value <0.05 compared to mock-transfected cells, Tukey test †P-value <0.05 between dCas9^{p300 Core} and dCas9^{VP64}, n=3 independent experiments, error bars: s.e.m.). The human MYOD locus is schematically depicted with corresponding gRNA locations in red. CE, MyoD core enhancer; DRR, MyoD distal regulatory region. FIG. 2B shows relative OCT4mRNA production in cells co-transfected with a pool of gRNAs targeted to the proximal and distal regulatory regions and dCas9^{VP64} or dCas9^{p300 Core}; promoter data from FIG. 1C (Tukey-test, *P-value <0.05 compared to mock-transfected cells, Tukey test †P-value <0.05 between dCas9^{p300 Core} and dCas9^{VP64}, n=3 independent experiments, error bars: s.e.m.). The human OCT4 locus is schematically depicted with corresponding gRNA locations in red. DE, Oct4 distal enhancer; PE, Oct4 proximal enhancer. FIG. 2C shows the human β-globin locus is schematically depicted with approximate locations of the hypersensitive site 2 (HS2) enhancer region and downstream genes (HBE, HBG, HBD, and HBB). Corresponding HS2 gRNA locations are shown in red. Relative mRNA production from distal genes in cells co-transfected with four gRNAs targeted to the HS2 enhancer and the indicated dCas9 proteins. Note logarithmic y-axis and dashed red line indicating background expression (Tukey test among conditions for each β-globin gene, †P-value <0.05, n=3 independent experiments, error bars: s.e.m.). n.s., not significant.

FIGS. 3A-3C show that dCas9^{p300 Core} targeted transcriptional activation is specific and robust. FIGS. 3A-3C show MA plots generated from DEseq2 analysis of genome-wide RNA-seq data from HEK293T cells transiently co-transfected with dCas9^{VP64} (FIG. 3A) dCas9^{p300 Core}(FIG. 3B) or dCas9^{p300 Core (D1399Y)} (FIG. 3C) and four IL1RN promoter-targeting gRNAs compared to HEK293T cells transiently co-transfected with dCas9 and four IL1RN promoter-targeting gRNAs. mRNAs corresponding to IL1RN isoforms are shown in blue and circled in each of FIGS. 3A-3C. Red labeled points in FIGS. 3B and 3C correspond to off-target transcripts significantly enriched after multiple hypothesis testing (KDR, (FDR=1.4×10⁻³); FAM49A, (FDR=0.04); p300, (FDR=1.7×10⁻⁴) in FIG. 3B; and p300, (FDR=4.4×10⁻¹⁰) in FIG. 3C.

FIGS. 4A-4D show that dCas9^{p300 Core} fusion protein acetylates chromatin at a targeted enhancer and corresponding downstream genes. FIG. 4A shows the region encompassing the human β-globin locus on chromosome 11 (5,304,000-5,268,000; GRCh37/hg19 assembly) is shown. HS2 gRNA target locations are indicated in red and ChIP-qPCR amplicon regions are depicted in black with corresponding green numbers. ENCODE/Broad Institute H3K27ac enrichment signal in K562 cells is shown for comparison. Magnified insets for the HS2 enhancer, HBE, and HBG1/2promoter regions are displayed below. FIGS. 4B-4D show H3K27ac ChIP-qPCR enrichment (relative to dCas9; red dotted line) at the HS2 enhancer, HBE promoter, and HBG1/2 promoters in cells co-transfected with four gRNAs targeted to the HS2 enhancer and the indicated dCas9 fusion protein. HBG ChIP amplicons 1 and 2 amplify redundant sequences at the HBG1 and HBG2 promoters (denoted by ‡). Tukey test among conditions for each ChIP-qPCR region, *P-value <0.05 (n=3 independent experiments, error bars: s.e.m.).

FIGS. 5A-5G show that dCas9^{p300 Core} fusion protein activates transcription of endogenous genes from regulatory regions with a single gRNA. Relative IL1RN (FIG. 5A), MYOD (FIG. 5B) or OCT4 (FIG. 5C) mRNA produced from cells co-transfected with dCas9^{p300 Core} or dCas9^{VP64} and gRNAs targeting respective promoters (n=3 independent experiments, error bars: s.e.m.). Relative MYOD (FIG. 5D) or OCT4 (FIG. 5E) mRNA produced from cells co-transfected with dCas9^{p300 Core} and indicated gRNAs targeting the indicated MYOD or OCT4 enhancers (n=3 independent experiments, error bars: s.e.m.). DRR, MYOD distal regulatory region; CE, MYOD core enhancer; PE, OCT4 proximal enhancer; DE, OCT4 distal enhancer. (Tukey test between dCas9^{p300 Core} and single OCT4 DE gRNAs compared to mock-transfected cells, *P-value <0.05, Tukey test among dCas9^{p300 Core} and OCT4 DE gRNAs compared to All, †P-value <0.05,). Relative HBE (FIG. 5F) or HBG (FIG. 5G) mRNA production in cells co-transfected with dCas9^{p300 Core} and the indicated gRNAs targeted to the HS2 enhancer (Tukey test between dCas9^{p300 Core} and single HS2 gRNAs compared to mock-transfected cells, *P-value <0.05, Tukey test among dCas9^{p300 Core} and HS2 single gRNAs compared to All, \P<0.05, n=3 independent experiments, error bars: s.e.m.). HS2, β-globin locus control region hypersensitive site 2; n.s., not significant using Tukey test.

FIGS. 6A-6H show that the p300 Core can be targeted to genomic loci by diverse programmable DNA-binding proteins. FIG. 6A shows schematic of the Neisseria meningitidis (Nm) dCas9 fusion proteins Nm-dCas9^{VP64} and Nm-dCas9^{p300 Core} Neisseria meningitidis dCas9 contains nuclease-inactivating mutations D16A, D587A, H588A, and N611A. FIG. 6B shows relative HBE mRNA in cells co-transfected with five individual or pooled (A-E) Nm gRNAs targeted to the HBE promoter and Nm-dCas9^{VP64} or Nm-dCas9^{p300 Core} FIGS. 6C-6D Relative HBE (FIG. 6C) or HBG (FIG. 6D) mRNA in cells co-transfected with five individual or pooled (A-E) Nm gRNAs targeted to the HS2 enhancer and Nm-dCas9^{VP64} or Nm-dCas9^{p300 Core} FIG. 6E shows schematic of TALEs with domains containing IL1RN-targeted repeat variable diresidues (Repeat Domain). FIG. 6F shows relative IL1RN mRNA in cells transfected with individual or pooled (A-D) IL1RN TALE^{VP64} or IL1RN TALE^{p300 Core} encoding plasmids. FIG. 6G shows schematic of ZF fusion proteins with zinc finger helices 1-6 (F1-F6) targeting the ICAM1 promoter. FIG. 6H shows relative ICAM1 mRNA in cells transfected with ICAM1 ZF^{VP64} or ICAM1 ZFP^{p300 Core} Tukey-test, *P-value <0.05 compared to mock-transfected control, n=3 independent experiments each, error bars: s.e.m. NLS, nuclear localization signal; HA, hemagglutinin tag; Bd, bromodomain; CH, cysteine-histidine-rich region; HAT, histone acetyltransferase domain.

FIGS. 7A-7C show dCas9^{p300 Core} mutant fusion protein activities. FIG. 7A shows schematic depiction of the WT dCas9^{p300 Core} fusion protein and p300 Core mutant derivatives. Relative locations of mutated amino acids are displayed as yellow bars within the p300 Core effector domain. FIG. 7B shows dCas9^{p300 Core} variants were transiently co-transfected with four IL1RN promoter gRNAs and were screened for hyperactivity¹ (amino acid 1645/1646 RR/EE and C1204R mutations) or hypoactivity (denoted by $) via mRNA production from the IL1RN locus (top panel, n=2 independent experiments, error bars: s.e.m.). Experiments were performed in duplicate with one well used for RNA isolation and the other for western blotting to validate expression (bottom panels). The nitrocellulose membrane was cut and incubated with α-FLAG primary antibody (top, Sigma-Aldrich cat. #F7425) or α-GAPDH (bottom, Cell Signaling Technology cat. #14C10) then α-Rabbit HRP secondary antibody (Sigma-Aldrich cat. #A6154). FIG. 7C shows full membranes from western blot shown in main text (FIG. 1). The nitrocellulose membrane was cut and incubated with α-FLAG primary antibody (top, Sigma-Aldrich cat. #F7425) or α-GAPDH (bottom, Cell Signaling Technology cat. #14C10) then α-Rabbit HRP secondary antibody (Sigma-Aldrich cat. #A6154). Membrane was imaged for the indicated durations after careful re-alignment of trimmed pieces.

FIG. 8 shows target gene activation is unaffected by overexpression of synthetic dCas9 fusion proteins.

FIGS. 9A-9E show a comparison of Sp. dCas9 and Nm. dCas9 gene induction from the HS2 enhancer with individual and pooled gRNAs. FIG. 9A shows schematic display of the human β-globin locus including Streptococcus pyogenes dCas9 (Sp. dCas9) and Neisseria meningitidis dCas9 (Nm. dCas9) gRNA locations at the HS2 enhancer. Layered transcription profiles scaled to a vertical viewing range of 8 from nine ENCODE cell lines (GM12878, H1-hESC, HeLa-S3, HepG2, HSMM, HUVEC, K562, NHEK, and NHLF) is shown in addition to ENCODE p300 binding peaks in K562, A549 (EtOH 0.02), HeLA-S3, and SKN_SH_RA cell lines. An ENCODE HEK293T DNase hypersensitive site (HEK293T DHS) is shown in the HS2 Enhancer inset. FIGS. 9B-9E shows relative transcriptional induction of HBE, HBG, HBD, and HBD transcripts from single and pooled Sp. dCas9 gRNAs (A-D) or single and pooled Nm. dCas9 gRNAs (A-E) in response to co-transfection with Sp. dCas9^{p300 Core} or Nm. dCas9^{p300 Core} respectively. gRNAs are tiled for each dCas9 ortholog corresponding to their location in GRCh37/hg19. Gray dashed line indicates background expression level in transiently co-transfected HEK293T cells. Note shared logarithmic scale among FIGS. 9B-9E. Numbers above bars in FIGS. 9B-9E indicate mean expression (n=at least 3 independent experiments, error bars. s.e.m.).

FIG. 10 shows that dCas9^{VP64} and dCas9^{p300 Core} induce H3K27ac enrichment at IL1RN gRNA-targeted chromatin.

FIGS. 11A-11C show a direct comparison of VP64 and p300 Core effector domains between TALE and dCas9 programmable DNA binding proteins. FIG. 11A shows the GRCh37/hg19 region encompassing the IL1RN transcription start site is shown schematically along with IL1RN TALE binding sites and dCas9 IL1RN gRNA target sites. FIG. 11B shows direct comparison of IL1RN activation in HEK293T cells when transfected with individual or pooled (A-D) IL1RN TALE^{VP64} fusion proteins or when co-transfected with dCas9^{VP64} and individual or pooled (A-D) IL1RN-targeting gRNAs. FIG. 11C shows direct comparison of IL1RN activation in HEK293T cells when transfected with individual or pooled (A-D) IL1RN TALE^{p300 Core} fusion proteins or when co-transfected with dCas9^{p300 Core} and individual or pooled (A-D) IL1RN-targeting gRNAs. Note shared logarithmic scale between FIG. 11B and FIG. 11C. Numbers above bars in FIGS. 11B and 11C indicate mean values. Tukey test, *P-value <0.05, n=at least 3 independent experiments, error bars: s.e.m.

FIGS. 12A-12B show TALE and ZF fusion protein expression. FIG. 12A shows Western blotting was carried out on cells transiently transfected with individual or pooled IL1RN TALE proteins. Nitrocellulose membranes were cut and probed with α-HA primary antibody (1:1000 dilution in TBST+5% Milk, top, Covance cat. #MMS-101P) or α-GAPDH (bottom, Cell Signaling Technology cat. #14C10) then α-Mouse HRP (Santa Cruz, sc-2005) or α-Rabbit HRP (Sigma-Aldrich cat. #A6154) secondary antibody, respectively. FIG. 12B shows Western blotting was carried out on cells transiently transfected with ICAM1 ZF-effector proteins and nitrocellulose membranes were cut and probed with α-FLAG primary antibody (top, Sigma-Aldrich cat. #F7425) or α-GAPDH (bottom, Cell Signaling Technology cat. #14C10) then α-Rabbit HRP secondary antibody (Sigma-Aldrich cat. #A6154). Red asterisk indicates non-specific band.

FIGS. 13A-13B show that dCas9^{p300 Core} and dCas9^{VP64} do not display synergy in transactivation. FIG. 13A shows dCas9^{p300 Core} was co-transfected at a 1:1 mass ratio to PL-SIN-EF1α-EGFP³ (GFP), dCas9, or dCas9^{VP64} with four IL1RN promoter gRNAs as indicated (n=2 independent experiments, error bars: s.e.m.). FIG. 13B shows dCas9^{p300 Core} was co-transfected at a 1:1 mass ratio to GFP, dCas9, or dCas9^{VP64} with four MYOD promoter gRNAs as indicated (n=2 independent experiments, error bars: s.e.m.). No significant differences were observed using Tukey's test (n.s.).

FIGS. 14A-14D show the underlying chromatin context of dCas9^{p300 Core} target loci. FIGS. 14A-14D show indicated loci along with associated Streptococcus pyogenes gRNAs used in this study at corresponding genomic locations in GRCh37/hg19. ENCODE HEK293T DNase hypersensitivity enrichment is shown (note changes in scale) along with regions of significant DNase hypersensitivity in HEK293T cells (“DHS”). In addition ENCODE master DNase clusters across 125 cell types are shown. Layered ENCODE H3K27ac and H3K4me3 enrichment across seven cell lines (GM12878, H1-hESC, HSMM, HUVEC, K562, NHEK, and NHLF) is also displayed and scaled to a vertical viewing range of 50 and 150 respectively. Endogenous p300 binding profiles are also indicated for each locus and respective cell line.

FIG. 14E shows an overview of the information provided in FIGS. 14A-14D.

FIGS. 15A-15J show the amino acid sequences of dCas9 constructs.

FIG. 16 shows the amino acid sequences of ICAM1 Zinc Finger¹⁰ effectors.

FIG. 17 shows gRNA design and screening.

FIG. 18 shows gRNA combination activation.

FIG. 19 shows Pax7 guide screening in 293 Ts.

FIG. 20 shows that gRNA19 localizes to a DHS.

FIG. 21 shows the relative quantity of FGF1A mRNA in 293 Ts with or without dCas9^{p300 Core}.

FIG. 22 shows expression levels of FGF1B and FGF1C in 293 Ts with dCas9^{p300 Core} dCas9^{VP64} or dCas9 alone.

FIG. 23 shows expression levels of FGF1A, FGF1B, and FGF1C in 293 Ts with dCas9^{p300 Core} dCas9^{VP64} or dCas9 alone.

### DETAILED DESCRIPTION

Disclosed herein are CRISPR/Cas9-based gene activation systems and methods of using said systems. The systems provide an easily programmable approach to facilitate robust control of the epigenome and downstream gene expression. The CRISPR/Cas9-based gene activation system includes a CRISPR/Cas9-based acetyltransferase, which is a fusion protein of a Cas9 protein and a protein having histone acetyltransferase activity, such as the catalytic histone acetyltransferase (HAT) core domain of the human E1A-associated protein p300. The Cas9 protein may not have nuclease activity. An example of a Cas9 protein where the nuclease activity has been abolished is dCas9. Recruitment of the acetyltransferase function by dCas9 and a gRNA to the genomic target site allow direct modulation of epigenetic structure, and thus provide an effective means of gene activation.

The disclosed CRISPR/Cas9-based acetyltransferase catalyzes acetylation of histone H3 lysine 27 at its target sites, leading to robust transcriptional activation of target genes from promoters and both proximal and distal enhancers. As disclosed herein, gene activation by these targeted acetyltransferases is highly specific across the genome. The CRISPR/Cas9-based acetyltransferase, which can be targeted to any site in the genome, is uniquely capable of activating distal regulatory elements. In contrast to conventional dCas9-based activators, the CRISPR/Cas9-based acetyltransferase effectively activates genes from enhancer regions and with individual or single guide RNAs.

### 1. Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

“Adeno-associated virus” or “AAV” as used interchangeably herein refers to a small virus belonging to the genus Dependovirus of the Parvoviridae family that infects humans and some other primate species. AAV is not currently known to cause disease and consequently the virus causes a very mild immune response.

“Chromatin” as used herein refers to an organized complex of chromosomal DNA associated with histones.

“Cis-regulatory elements” or “CREs” as used interchangeably herein refers to regions of non-coding DNA which regulate the transcription of nearby genes. CREs are found in the vicinity of the gene, or genes, they regulate. CREs typically regulate gene transcription by functioning as binding sites for transcription factors. Examples of CREs include promoters and enhancers.

“Clustered Regularly Interspaced Short Palindromic Repeats” and “CRISPRs”, as used interchangeably herein refers to loci containing multiple short direct repeats that are found in the genomes of approximately 40% of sequenced bacteria and 90% of sequenced archaea.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The coding sequence may be codon optimize.

“Complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

“Endogenous gene” as used herein refers to a gene that originates from within an organism, tissue, or cell. An endogenous gene is native to a cell, which is in its normal genomic and chromatin context, and which is not heterologous to the cell. Such cellular genes include, e.g., animal genes, plant genes, bacterial genes, protozoal genes, fungal genes, mitochondrial genes, and chloroplastic genes.

“Enhancer” as used herein refers to non-coding DNA sequences containing multiple activator and repressor binding sites. Enhancers range from 200 bp to 1 kb in length and may be either proximal, 5′ upstream to the promoter or within the first intron of the regulated gene, or distal, in introns of neighboring genes or intergenic regions far away from the locus. Through DNA looping, active enhancers contact the promoter dependently of the core DNA binding motif promoter specificity. 4 to 5 enhancers may interact with a promoter. Similarly, enhancers may regulate more than one gene without linkage restriction and may “skip” neighboring genes to regulate more distant ones. Transcriptional regulation may involve elements located in a chromosome different to one where the promoter resides. Proximal enhancers or promoters of neighboring genes may serve as platforms to recruit more distal elements.

“Fusion protein” as used herein refers to a chimeric protein created through the joining of two or more genes that originally coded for separate proteins. The translation of the fusion gene results in a single polypeptide with functional properties derived from each of the original proteins.

“Genetic construct” as used herein refers to the DNA or RNA molecules that comprise a nucleotide sequence that encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Histone acetyltransferases” or “HATs” are used interchangeably herein refers to enzymes that acetylate conserved lysine amino acids on histone proteins by transferring an acetyl group from acetyl CoA to form ε-N-acetyllysine. DNA is wrapped around histones, and, by transferring an acetyl group to the histones, genes can be turned on and off. In general, histone acetylation increases gene expression as it is linked to transcriptional activation and associated with euchromatin. Histone acetyltransferases can also acetylate non-histone proteins, such as nuclear receptors and other transcription factors to facilitate gene expression.

“Identical” or “identity” as used herein in the context of two or more nucleic acids or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

“Operably linked” as used herein means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function. [0056]“p300 protein,” “EP300,” or “E1A binding protein p300” as used interchangeably herein refers to the adenovirus E1A-associated cellular p300 transcriptional co-activator protein encoded by the EP300 gene. p300 is a highly conserved acetyltransferase involved in a wide range of cellular processes. p300 functions as a histone acetyltransferase that regulates transcription via chromatin remodeling and is involved with the processes of cell proliferation and differentiation.

“Promoter” as used herein means a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

“Target enhancer” as used herein refers to enhancer that is targeted by a gRNA and CRISPR/Cas9-based gene activation system. The target enhancer may be within the target region.

“Target gene” as used herein refers to any nucleotide sequence encoding a known or putative gene product. The target gene includes the regulatory regions, such as the promoter and enhancer regions, the transcribed regions, which include the coding regions, and other function sequence regions.

“Target region” as used herein refers to a cis-regulatory region or a trans-regulatory region of a target gene to which the guide RNA is designed to recruit the CRISPR/Cas9-based gene activation system to modulate the epigenetic structure and allow the activation of gene expression of the target gene.

“Target regulatory element” as used herein refers to a regulatory element that is targeted by a gRNA and CRISPR/Cas9-based gene activation system. The target regulatory element may be within the target region.

“Transcribed region” as used herein refers to the region of DNA that is transcribed into single-stranded RNA molecule, known as messenger RNA, resulting in the transfer of genetic information from the DNA molecule to the messenger RNA. During transcription, RNA polymerase reads the template strand in the 3′ to 5′ direction and synthesizes the RNA from 5′ to 3′. The mRNA sequence is complementary to the DNA strand.

“Transcriptional Start Site” or “TSS” as used interchangeably herein refers to the first nucleotide of a transcribed DNA sequence where RNA polymerase begins synthesizing the RNA transcript.

“Transgene” as used herein refers to a gene or genetic material containing a gene sequence that has been isolated from one organism and is introduced into a different organism. This non-native segment of DNA may retain the ability to produce RNA or protein in the transgenic organism, or it may alter the normal function of the transgenic organism's genetic code. The introduction of a transgene has the potential to change the phenotype of an organism.

“Trans-regulatory elements” as used herein refers to regions of non-coding DNA which regulate the transcription of genes distant from the gene from which they were transcribed. Trans-regulatory elements may be on the same or different chromosome from the target gene.

“Variant” used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes may be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes may be substituted and still retain protein function. In one aspect, amino acids having hydropathic indexes of 2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Vector” as used herein means a nucleic acid sequence containing an origin of replication. A vector may be a viral vector, bacteriophage, bacterial artificial chromosome or yeast artificial chromosome. A vector may be a DNA or RNA vector. A vector may be a self-replicating extrachromosomal vector, and preferably, is a DNA plasmid. For example, the vector may encode a CRISPR/Cas9-based acetyltransferase having an amino acid sequence of SEQ ID NO: 140, 141, or 149 and/or at least one gRNA nucleotide sequence of any one of SEQ ID NOs: 23-73, 188-223, or 224-254.

### 2. CRISPR/Cas9-Based Gene Activation System

Provided herein are CRISPR/Cas9-based gene activation systems for use in activating gene expression of a target gene. The CRISPR/Cas9-based gene activation system includes a fusion protein of a Cas9 protein that does not have nuclease activity, such as dCas9, and a histone acetyltransferase or histone acetyltransferase effector domain. Histone acetylation, carried out by histone acetyltransferases (HATs), plays a fundamental role in regulating chromatin dynamics and transcriptional regulation. The histone acetyltransferase protein releases DNA from its heterochromatin state and allows for continued and robust gene expression by the endogenous cellular machinery. The recruitment of an acetyltransferase by dCas9 to a genomic target site may directly modulate epigenetic structure.

The CRISPR/Cas9-based gene activation system may catalyze acetylation of histone H3 lysine 27 at its target sites, leading to robust transcriptional activation of target genes from promoters and proximal and distal enhancers. The CRISPR/Cas9-based gene activation system is highly specific and may be guided to the target gene using as few as one guide RNA. The CRISPR/Cas9-based gene activation system may activate the expression of one gene or a family of genes by targeting enhancers at distant locations in the genome.

### a) CRISPR System

The CRISPR system is a microbial nuclease system involved in defense against invading phages and plasmids that provides a form of acquired immunity. The CRISPR loci in microbial hosts contain a combination of CRISPR-associated (Cas) genes as well as non-coding RNA elements capable of programming the specificity of the CRISPR-mediated nucleic acid cleavage. Short segments of foreign DNA, called spacers, are incorporated into the genome between CRISPR repeats, and serve as a ‘memory’ of past exposures. Cas9 forms a complex with the 3′ end of the single guide RNA (“sgRNA”), and the protein-RNA pair recognizes its genomic target by complementary base pairing between the 5′ end of the sgRNA sequence and a predefined 20 bp DNA sequence, known as the protospacer. This complex is directed to homologous loci of pathogen DNA via regions encoded within the CRISPR RNA (“crRNA”), i.e., the protospacers, and protospacer-adjacent motifs (PAMs) within the pathogen genome. The non-coding CRISPR array is transcribed and cleaved within direct repeats into short crRNAs containing individual spacer sequences, which direct Cas nucleases to the target site (protospacer). By simply exchanging the 20 bp recognition sequence of the expressed chimeric sgRNA, the Cas9 nuclease can be directed to new genomic targets. CRISPR spacers are used to recognize and silence exogenous genetic elements in a manner analogous to RNAi in eukaryotic organisms.

Three classes of CRISPR systems (Types I, II and III effector systems) are known. The Type II effector system carries out targeted DNA double-strand break in four sequential steps, using a single effector enzyme, Cas9, to cleave dsDNA. Compared to the Type I and Type III effector systems, which require multiple distinct effectors acting as a complex, the Type II effector system may function in alternative contexts such as eukaryotic cells. The Type II effector system consists of a long pre-crRNA, which is transcribed from the spacer-containing CRISPR locus, the Cas9 protein, and a tracrRNA, which is involved in pre-crRNA processing. The tracrRNAs hybridize to the repeat regions separating the spacers of the pre-crRNA, thus initiating dsRNA cleavage by endogenous RNase III. This cleavage is followed by a second cleavage event within each spacer by Cas9, producing mature crRNAs that remain associated with the tracrRNA and Cas9, forming a Cas9:crRNA-tracrRNA complex.

An engineered form of the Type II effector system of Streptococcus pyogenes was shown to function in human cells for genome engineering. In this system, the Cas9 protein was directed to genomic target sites by a synthetically reconstituted “guide RNA” (“gRNA”, also used interchangeably herein as a chimeric sgRNA, which is a crRNA-tracrRNA fusion that obviates the need for RNase III and crRNA processing in general.

The Cas9:crRNA-tracrRNA complex unwinds the DNA duplex and searches for sequences matching the crRNA to cleave. Target recognition occurs upon detection of complementarity between a “protospacer” sequence in the target DNA and the remaining spacer sequence in the crRNA. Cas9 mediates cleavage of target DNA if a correct protospacer-adjacent motif (PAM) is also present at the 3′ end of the protospacer. For protospacer targeting, the sequence must be immediately followed by the protospacer-adjacent motif (PAM), a short sequence recognized by the Cas9 nuclease that is required for DNA cleavage. Different Type II systems have differing PAM requirements. The S. pyogenes CRISPR system may have the PAM sequence for this Cas9 (SpCas9) as 5′-NRG-3′, where R is either A or G, and characterized the specificity of this system in human cells. A unique capability of the CRISPR/Cas9 system is the straightforward ability to simultaneously target multiple distinct genomic loci by co-expressing a single Cas9 protein with two or more sgRNAs. For example, the Streptococcus pyogenes Type II system naturally prefers to use an “NGG” sequence, where “N” can be any nucleotide, but also accepts other PAM sequences, such as “NAG” in engineered systems (Hsu et al., Nature Biotechnology (2013) doi:10.1038/nbt.2647). Similarly, the Cas9 derived from Neisseria meningitidis (NmCas9) normally has a native PAM of NNNNGATT, but has activity across a variety of PAMs, including a highly degenerate NNNNGNNN PAM (Esvelt et al. Nature Methods (2013) doi:10.1038/nmeth.2681).

### b) Cas9

The CRISPR/Cas9-based gene activation system may include a Cas9 protein or a Cas9 fusion protein. Cas9 protein is an endonuclease that cleaves nucleic acid and is encoded by the CRISPR loci and is involved in the Type II CRISPR system. The Cas9 protein may be from any bacterial or archaea species, such as Streptococcus pyogenes, Streptococcus thermophiles, or Neisseria meningitides. The Cas9 protein may be mutated so that the nuclease activity is inactivated. In some embodiments, an inactivated Cas9 protein from Streptococcus pyogenes (iCas9, also referred to as “dCas9”; SEQ ID NO: 1) may be used. As used herein, “iCas9” and “dCas9” both refer to a Cas9 protein that has the amino acid substitutions D10A and H840A and has its nuclease activity inactivated. In some embodiments, an inactivated Cas9 protein from Neisseria meningitides, such as NmCas9 having an amino acid sequence of SEQ ID NO: 10, may be used.

### c) Histone Acetyltransferase (HAT) Protein

The CRISPR/Cas9-based gene activation system may include a histone acetyltransferase protein, such as a p300 protein, CREB binding protein (CBP; an analog of p300), GCN5, or PCAF, or fragment thereof. The p300 protein regulates the activity of many genes in tissues throughout the body. The p300 protein plays a role in regulating cell growth and division, prompting cells to mature and assume specialized functions (differentiate) and preventing the growth of cancerous tumors. The p300 protein may activate transcription by connecting transcription factors with a complex of proteins that carry out transcription in the cell's nucleus. The p300 protein also functions as a histone acetyltransferase that regulates transcription via chromatin remodeling.

The histone acetyltransferase protein may include a human p300 protein or a fragment thereof. The histone acetyltransferase protein may include a wild-type human p300 protein or a mutant human p300 protein, or fragments thereof. The histone acetyltransferase protein may include the core lysine-acetyltransferase domain of the human p300 protein, i.e., the p300 HAT Core (also known as “p300 Core”). In some embodiments, the histone acetyltransferase protein includes an amino acid sequence of SEQ ID NO: 2 or 3.

i) dCas9^{p300 Core}

The CRISPR/Cas9-based gene activation system may include a histone acetylation effector domain. The histone acetylation effector domain may be the catalytic histone acetyltransferase (HAT) core domain of the human E1A-associated protein p300 (also referred to herein as “p300 Core”). In some embodiments, the p300 Core includes amino acids 1048-1664 of SEQ ID NO: 2 (i.e., SEQ ID NO: 3). In some embodiments, the CRISPR/Cas9-based gene activation system includes a dCas9^{p300 Core} fusion protein of SEQ ID NO: 141 or an Nm-dCas9^{p300 Core} fusion protein of SEQ ID NO: 149. The p300 Core acetylates lysine 27 on histone H3 (H3K27ac) and may provide H3K27ac enrichment.

The dCas9^{p300 Core} fusion protein is a potent and easily programmable tool to synthetically manipulate acetylation at targeted endogenous loci, leading to regulation of proximal and distal enhancer-regulated genes. The fusion of the catalytic core domain of p300 to dCas9 may result in substantially higher transactivation of downstream genes than the direct fusion of full-length p300 protein despite robust protein expression. The dCas9^{p300 Core} fusion protein may also exhibit an increased transactivation capacity relative to dCas9^{VP64}, including in the context of the Nm-dCas9 scaffold, especially at distal enhancer regions, at which dCas9^{VP64} displayed little, if any, measurable downstream transcriptional activity. Additionally, the dCas9^{p300 Core} displays precise and robust genome-wide transcriptional specificity. dCas9^{p300 Core} may be capable of potent transcriptional activation and co-enrichment of acetylation at promoters targeted by the epigenetically modified enhancer.

The dCas9^{p300 Core} may activate gene expression through a single gRNA that target and bind a promoters and/or a characterized enhancer. This technology also affords the ability to synthetically transactivate distal genes from putative and known regulatory regions and simplifies transactivation via the application of a single programmable effector and single target site. These capabilities allow multiplexing to target several promoters and/or enhancers simultaneously. The mammalian origin of p300 may provide advantages over virally-derived effector domains for in vivo applications by minimizing potential immunogenicity.

d) gRNA

The CRISPR/Cas9-based gene activation system may include at least one gRNA that targets a nucleic acid sequence. The gRNA provides the targeting of the CRISPR/Cas9-based gene activation system. The gRNA is a fusion of two noncoding RNAs: a crRNA and a tracrRNA. The sgRNA may target any desired DNA sequence by exchanging the sequence encoding a 20 bp protospacer which confers targeting specificity through complementary base pairing with the desired DNA target. gRNA mimics the naturally occurring crRNA:tracrRNA duplex involved in the Type II Effector system. This duplex, which may include, for example, a 42-nucleotide crRNA and a 75-nucleotide tracrRNA, acts as a guide for the Cas9.

The gRNA may target and bind a target region of a target gene. The target region may be a cis-regulatory region or trans-regulatory region of a target gene. In some embodiments, the target region is a distal or proximal cis-regulatory region of the target gene. The gRNA may target and bind a cis-regulatory region or trans-regulatory region of a target gene. In some embodiments, the gRNA may target and bind an enhancer region, a promoter region, or a transcribed region of a target gene. For example, the gRNA may target and bind the target region is at least one of HS2 enhancer of the human β-globin locus, distal regulatory region (DRR) of the MYOD gene, core enhancer (CE) of the MYOD gene, proximal (PE) enhancer region of the OCT4 gene, or distal (DE) enhancer region of the OCT4 gene. In some embodiments, the target region may be a viral promoter, such as an HIV promoter.

The target region may include a target enhancer or a target regulatory element. In some embodiments, the target enhancer or target regulatory element controls the gene expression of several target genes. In some embodiments, the target enhancer or target regulatory element controls a cell phenotype that involves the gene expression of one or more target genes. In some embodiments, the identity of one or more of the target genes is known. In some embodiments, the identity of one or more of the target genes is unknown. The CRISPR/Cas9-based gene activation system allows the determination of the identity of these unknown genes that are involved in a cell phenotype. Examples of cell phenotypes include, but not limited to, T-cell phenotype, cell differentiation, such as hematopoietic cell differentiation, oncogenesis, immunomodulation, cell response to stimuli, cell death, cell growth, drug resistance, or drug sensitivity.

In some embodiments, at least one gRNA may target and bind a target enhancer or target regulatory element, whereby the expression of one or more genes is activated. For example, between 1 gene and 20 genes, between 1 gene and 15 genes, between 1 gene and 10 genes, between 1 gene and 5 genes, between 2 genes and 20 genes, between 2 genes and 15 genes, between 2 genes and 10 genes, between 2 genes and 5 genes, between 5 genes and 20 genes, between 5 genes and 15 genes, or between 5 genes and 10 genes are activated by at least one gRNA. In some embodiments, at least 1 gene, at least 2 genes, at least 3 genes, at least 4 genes, at least 5 gene, at least 6 genes, at least 7 genes, at least 8 genes, at least 9 gene, at least 10 genes, at least 11 genes, at least 12 genes, at least 13 gene, at least 14 genes, at least 15 genes, or at least 20 genes are activated by at least one gRNA.

The CRISPR/Cas9-based gene activation system may activate genes at both proximal and distal locations relative the transcriptional start site (TSS). The CRISPR/Cas9-based gene activation system may target a region that is at least about 1 base pair to about 100,000 base pairs, at least about 100 base pairs to about 100,000 base pairs, at least about 250 base pairs to about 100,000 base pairs, at least about 500 base pairs to about 100,000 base pairs, at least about 1,000 base pairs to about 100,000 base pairs, at least about 2,000 base pairs to about 100,000 base pairs, at least about 5,000 base pairs to about 100,000 base pairs, at least about 10,000 base pairs to about 100,000 base pairs, at least about 20,000 base pairs to about 100,000 base pairs, at least about 50,000 base pairs to about 100,000 base pairs, at least about 75,000 base pairs to about 100,000 base pairs, at least about 1 base pair to about 75,000 base pairs, at least about 100 base pairs to about 75,000 base pairs, at least about 250 base pairs to about 75,000 base pairs, at least about 500 base pairs to about 75,000 base pairs, at least about 1,000 base pairs to about 75,000 base pairs, at least about 2,000 base pairs to about 75,000 base pairs, at least about 5,000 base pairs to about 75,000 base pairs, at least about 10,000 base pairs to about 75,000 base pairs, at least about 20,000 base pairs to about 75,000 base pairs, at least about 50,000 base pairs to about 75,000 base pairs, at least about 1 base pair to about 50,000 base pairs, at least about 100 base pairs to about 50,000 base pairs, at least about 250 base pairs to about 50,000 base pairs, at least about 500 base pairs to about 50,000 base pairs, at least about 1,000 base pairs to about 50,000 base pairs, at least about 2,000 base pairs to about 50,000 base pairs, at least about 5,000 base pairs to about 50,000 base pairs, at least about 10,000 base pairs to about 50,000 base pairs, at least about 20,000 base pairs to about 50,000 base pairs, at least about 1 base pair to about 25,000 base pairs, at least about 100 base pairs to about 25,000 base pairs, at least about 250 base pairs to about 25,000 base pairs, at least about 500 base pairs to about 25,000 base pairs, at least about 1,000 base pairs to about 25,000 base pairs, at least about 2,000 base pairs to about 25,000 base pairs, at least about 5,000 base pairs to about 25,000 base pairs, at least about 10,000 base pairs to about 25,000 base pairs, at least about 20,000 base pairs to about 25,000 base pairs, at least about 1 base pair to about 10,000 base pairs, at least about 100 base pairs to about 10,000 base pairs, at least about 250 base pairs to about 10,000 base pairs, at least about 500 base pairs to about 10,000 base pairs, at least about 1,000 base pairs to about 10,000 base pairs, at least about 2,000 base pairs to about 10,000 base pairs, at least about 5,000 base pairs to about 10,000 base pairs, at least about 1 base pair to about 5,000 base pairs, at least about 100 base pairs to about 5,000 base pairs, at least about 250 base pairs to about 5,000 base pairs, at least about 500 base pairs to about 5,000 base pairs, at least about 1,000 base pairs to about 5,000 base pairs, or at least about 2,000 base pairs to about 5,000 base pairs upstream from the TSS. The CRISPR/Cas9-based gene activation system may target a region that is at least about 1 base pair, at least about 100 base pairs, at least about 500 base pairs, at least about 1,000 base pairs, at least about 1,250 base pairs, at least about 2,000 base pairs, at least about 2,250 base pairs, at least about 2,500 base pairs, at least about 5,000 base pairs, at least about 10,000 base pairs, at least about 11,000 base pairs, at least about 20,000 base pairs, at least about 30,000 base pairs, at least about 46,000 base pairs, at least about 50,000 base pairs, at least about 54,000 base pairs, at least about 75,000 base pairs, or at least about 100,000 base pairs upstream from the TSS.

The CRISPR/Cas9-based gene activation system may target a region that is at least about 1 base pair to at least about 500 base pairs, at least about 1 base pair to at least about 250 base pairs, at least about 1 base pair to at least about 200 base pairs, at least about 1 base pair to at least about 100 base pairs, at least about 50 base pairs to at least about 500 base pairs, at least about 50 base pairs to at least about 250 base pairs at least about 50 base pairs to at least about 200 base pairs, at least about 50 base pairs to at least about 100 base pairs, at least about 100 base pairs to at least about 500 base pairs, at least about 100 base pairs to at least about 250 base pairs, or at least about 100 base pairs to at least about 200 base pairs downstream from the TSS. The CRISPR/Cas9-based gene activation system may target a region that is at least about 1 base pair, at least about 2 base pairs, at least about 3 base pairs, at least about 4 base pairs, at least about 5 base pairs, at least about 10 base pairs, at least about 15 base pairs, at least about 20 base pairs, at least about 25 base pairs, at least about 30 base pairs, at least about 40 base pairs, at least about 50 base pairs, at least about 60 base pairs, at least about 70 base pairs, at least about 80 base pairs, at least about 90 base pairs, at least about 100 base pairs, at least about 110 base pairs, at least about 120, at least about 130, at least about 140 base pairs, at least about 150 base pairs, at least about 160 base pairs, at least about 170 base pairs, at least about 180 base pairs, at least about 190 base pairs, at least about 200 base pairs, at least about 210 base pairs, at least about 220, at least about 230, at least about 240 base pairs, or at least about 250 base pairs downstream from the TSS.

In some embodiments, the CRISPR/Cas9-based gene activation system may target and bind a target region that is on the same chromosome as the target gene but more than 100,000 base pairs upstream or more than 250 base pairs downstream from the TSS. In some embodiments, the CRISPR/Cas9-based gene activation system may target and bind a target region that is on a different chromosome from the target gene.

The CRISPR/Cas9-based gene activation system may use gRNA of varying sequences and lengths. The gRNA may comprise a complementary polynucleotide sequence of the target DNA sequence followed by NGG. The gRNA may comprise a “G” at the 5′ end of the complementary polynucleotide sequence. The gRNA may comprise at least a 10 base pair, at least a 11 base pair, at least a 12 base pair, at least a 13 base pair, at least a 14 base pair, at least a 15 base pair, at least a 16 base pair, at least a 17 base pair, at least a 18 base pair, at least a 19 base pair, at least a 20 base pair, at least a 21 base pair, at least a 22 base pair, at least a 23 base pair, at least a 24 base pair, at least a 25 base pair, at least a 30 base pair, or at least a 35 base pair complementary polynucleotide sequence of the target DNA sequence followed by NGG. The gRNA may target at least one of the promoter region, the enhancer region or the transcribed region of the target gene. The gRNA may include a nucleic acid sequence of at least one of SEQ ID NOs: 23-73, 188-223, or 224-254.

The CRISPR/Cas9-based gene activation system may include at least 1 gRNA, at least 2 different gRNAs, at least 3 different gRNAs at least 4 different gRNAs, at least 5 different gRNAs, at least 6 different gRNAs, at least 7 different gRNAs, at least 8 different gRNAs, at least 9 different gRNAs, or at least 10 different gRNAs. The CRISPR/Cas9-based gene activation system may include between at least 1 gRNA to at least 10 different gRNAs, at least 1 gRNA to at least 8 different gRNAs, at least 1 gRNA to at least 4 different gRNAs, at least 2 gRNA to at least 10 different gRNAs, at least 2 gRNA to at least 8 different gRNAs, at least 2 different gRNAs to at least 4 different gRNAs, at least 4 gRNA to at least 10 different gRNAs, or at least 4 different gRNAs to at least 8 different gRNAs.

### 3. Target Genes

The CRISPR/Cas9-based gene activation system may be designed to target and activate the expression of any target gene. The target gene may be an endogenous gene, a transgene, or a viral gene in a cell line. In some embodiments, the target region is located on a different chromosome as the target gene. In some embodiments, the CRISPR/Cas9-based gene activation system may include more than 1 gRNA. In some embodiments, the CRISPR/Cas9-based gene activation system may include more than 1 different gRNAs. In some embodiments, the different gRNAs bind to different target regions. For example, the different gRNAs may bind to target regions of different target genes and the expression of two or more target genes are activated.

In some embodiments, the CRISPR/Cas9-based gene activation system may activate between about one target gene to about ten target genes, about one target genes to about five target genes, about one target genes to about four target genes, about one target genes to about three target genes, about one target genes to about two target genes, about two target gene to about ten target genes, about two target genes to about five target genes, about two target genes to about four target genes, about two target genes to about three target genes, about three target genes to about ten target genes, about three target genes to about five target genes, or about three target genes to about four target genes. In some embodiments, the CRISPR/Cas9-based gene activation system may activate at least one target gene, at least two target genes, at least three target genes, at least four target genes, at least five target genes, or at least ten target genes. For example, the may target the hypersensitive site 2 (HS2) enhancer region of the human β-globin locus and activate downstream genes (HBE, HBG, HBD and HBB).

In some embodiments, the CRISPR/Cas9-based gene activation system induces the gene expression of a target gene by at least about 1 fold, at least about 2 fold, at least about 3 fold, at least about 4 fold, at least about 5 fold, at least about 6 fold, at least about 7 fold, at least about 8 fold, at least about 9 fold, at least about 10 fold, at least 15 fold, at least 20 fold, at least 30 fold, at least 40 fold, at least 50 fold, at least 60 fold, at least 70 fold, at least 80 fold, at least 90 fold, at least 100 fold, at least about 110 fold, at least 120 fold, at least 130 fold, at least 140 fold, at least 150 fold, at least 160 fold, at least 170 fold, at least 180 fold, at least 190 fold, at least 200 fold, at least about 300 fold, at least 400 fold, at least 500 fold, at least 600 fold, at least 700 fold, at least 800 fold, at least 900 fold, or at least 1000 fold compared to a control level of gene expression. A control level of gene expression of the target gene may be the level of gene expression of the target gene in a cell that is not treated with any CRISPR/Cas9-based gene activation system

The target gene may be a mammalian gene. For example, the CRISPR/Cas9-based gene activation system may target a mammalian gene, such as IL1RN, MYOD1, OCT4, HBE, HBG, HBD, HBB, MYOCD (Myocardin), PAX7 (Paired box protein Pax-7), FGF (fibroblast growth factor-1) genes, such as FGF1A, FGF1B, and FGF1C. Other target genes include, but not limited to, Atf3, Axud1, Btg2, c-Fos, c-Jun, Cxcl1, Cxcl2, Edn1, Ereg, Fos, Gadd45b, Ier2, Ier3, Ifrd1, I11b, Il6, Irf1, Junb, Lif, Nfkbia, Nfkbiz, Ptgs2, Slc25a25, Sqstm1, Tieg, Tnf, Tnfaip3, Zfp36, Birc2, Ccl2, Ccl20, Ccl7, Cebpd, Ch25h, CSF1, Cx3cl1, Cxcl10, Cxcl5, Gch, Icam1, Ifi47, Ifngr2, Mmp10, Nfkbie, Npal1, p21, Relb, Ripk2, Rnd1, Slpr3, Stx11, Tgtp, Tlr2, Tmem140, Tnfaip2, Tnfrsf6, Vcam1, 1110004C05Rik (GenBank accession number BC010291), Abca1, AI561871 (GenBank accession number B1143915), AI882074 (GenBank accession number BB730912), Arts1, AW049765 (GenBank accession number BC026642.1), C3, Casp4, Cel5, Ccl9, Cdsn, Enpp2, Gbp2, H2-D1, H2-K, H2-L, Ifit1, Ii, Il13ra1, Il1rl1, Lcn2, Lhfpl2, LOC677168 (GenBank accession number AK019325), Mmp13, Mmp3, Mt2, Naf1, Ppicap, Prnd, Psmb10, Saa3, Serpina3g, Serpinf1, Sod3, Stat1, Tapbp, U90926 (GenBank accession number NM_020562), Ubd, A2AR (Adenosine A2A receptor), B7-H3 (also called CD276), B7-H4 (also called VTCN1), BTLA (B and T Lymphocyte Attenuator; also called CD272), CTLA-4 (Cytotoxic T-Lymphocyte-Associated protein 4; also called CD152), IDO (Indoleamine 2,3-dioxygenase) KIR (Killer-cell Immunoglobulin-like Receptor), LAG3 (Lymphocyte Activation Gene-3), PD-1 (Programmed Death 1 (PD-1) receptor), TIM-3 (T-cell Immunoglobulin domain and Mucin domain 3), and VISTA (V-domain Ig suppressor of T cell activation).

### 4. Compositions for Gene Activation

The present invention is directed to a composition for activating gene expression of a target gene, target enhancer, or target regulatory element in a cell or subject. The composition may include the CRISPR/Cas9-based gene activation system, as disclosed above. The composition may also include a viral delivery system. For example, the viral delivery system may include an adeno-associated virus vector or a modified lentiviral vector.

Methods of introducing a nucleic acid into a host cell are known in the art, and any known method can be used to introduce a nucleic acid (e.g., an expression construct) into a cell. Suitable methods include, include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery, and the like. In some embodiments, the composition may be delivered by mRNA delivery and ribonucleoprotein (RNP) complex delivery.

### a) Constructs and Plasmids

The compositions, as described above, may comprise genetic constructs that encodes the CRISPR/Cas9-based gene activation system, as disclosed herein. The genetic construct, such as a plasmid or expression vector, may comprise a nucleic acid that encodes the CRISPR/Cas9-based gene activation system, such as the CRISPR/Cas9-based acetyltransferase and/or at least one of the gRNAs. The compositions, as described above, may comprise genetic constructs that encodes the modified AAV vector and a nucleic acid sequence that encodes the CRISPR/Cas9-based gene activation system, as disclosed herein. The genetic construct, such as a plasmid, may comprise a nucleic acid that encodes the CRISPR/Cas9-based gene activation system. The compositions, as described above, may comprise genetic constructs that encodes a modified lentiviral vector. The genetic construct, such as a plasmid, may comprise a nucleic acid that encodes the CRISPR/Cas9-based acetyltransferase and at least one sgRNA. The genetic construct may be present in the cell as a functioning extrachromosomal molecule. The genetic construct may be a linear minichromosome including centromere, telomeres or plasmids or cosmids.

The genetic construct may also be part of a genome of a recombinant viral vector, including recombinant lentivirus, recombinant adenovirus, and recombinant adenovirus associated virus. The genetic construct may be part of the genetic material in attenuated live microorganisms or recombinant microbial vectors which live in cells. The genetic constructs may comprise regulatory elements for gene expression of the coding sequences of the nucleic acid. The regulatory elements may be a promoter, an enhancer, an initiation codon, a stop codon, or a polyadenylation signal.

The nucleic acid sequences may make up a genetic construct that may be a vector. The vector may be capable of expressing the fusion protein, such as the CRISPR/Cas9-based gene activation system, in the cell of a mammal. The vector may be recombinant. The vector may comprise heterologous nucleic acid encoding the fusion protein, such as the CRISPR/Cas9-based gene activation system. The vector may be a plasmid. The vector may be useful for transfecting cells with nucleic acid encoding the CRISPR/Cas9-based gene activation system, which the transformed host cell is cultured and maintained under conditions wherein expression of the CRISPR/Cas9-based gene activation system takes place.

Coding sequences may be optimized for stability and high levels of expression. In some instances, codons are selected to reduce secondary structure formation of the RNA such as that formed due to intramolecular bonding.

The vector may comprise heterologous nucleic acid encoding the CRISPR/Cas9-based gene activation system and may further comprise an initiation codon, which may be upstream of the CRISPR/Cas9-based gene activation system coding sequence, and a stop codon, which may be downstream of the CRISPR/Cas9-based gene activation system coding sequence. The initiation and termination codon may be in frame with the CRISPR/Cas9-based gene activation system coding sequence. The vector may also comprise a promoter that is operably linked to the CRISPR/Cas9-based gene activation system coding sequence. The CRISPR/Cas9-based gene activation system may be under the light-inducible or chemically inducible control to enable the dynamic control of gene activation in space and time. The promoter operably linked to the CRISPR/Cas9-based gene activation system coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human ubiquitin C (hUbC), human actin, human myosin, human hemoglobin, human muscle creatine, or human metalothionein. The promoter may also be a tissue specific promoter, such as a muscle or skin specific promoter, natural or synthetic. Examples of such promoters are described in US Patent Application Publication No. US20040175727, the contents of which are incorporated herein in its entirety.

The vector may also comprise a polyadenylation signal, which may be downstream of the CRISPR/Cas9-based gene activation system. The polyadenylation signal may be a SV40 polyadenylation signal, LTR polyadenylation signal, bovine growth hormone (bGH) polyadenylation signal, human growth hormone (hGH) polyadenylation signal, or human (3-globin polyadenylation signal. The SV40 polyadenylation signal may be a polyadenylation signal from a pCEP4 vector (Invitrogen, San Diego, CA).

The vector may also comprise an enhancer upstream of the CRISPR/Cas9-based gene activation system, i.e., the CRISPR/Cas9-based acetyltransferase coding sequence or sgRNAs. The enhancer may be necessary for DNA expression. The enhancer may be human actin, human myosin, human hemoglobin, human muscle creatine or a viral enhancer such as one from CMV, HA, RSV or EBV. Polynucleotide function enhancers are described in U.S. Pat. Nos. 5,593,972, 5,962,428, and WO94/016737, the contents of each are fully incorporated by reference. The vector may also comprise a mammalian origin of replication in order to maintain the vector extrachromosomally and produce multiple copies of the vector in a cell. The vector may also comprise a regulatory sequence, which may be well suited for gene expression in a mammalian or human cell into which the vector is administered. The vector may also comprise a reporter gene, such as green fluorescent protein (“GFP”) and/or a selectable marker, such as hygromycin (“Hygro”).

The vector may be expression vectors or systems to produce protein by routine techniques and readily available starting materials including Sambrook et al., Molecular Cloning and Laboratory Manual, Second Ed., Cold Spring Harbor (1989), which is incorporated fully by reference. In some embodiments the vector may comprise the nucleic acid sequence encoding the CRISPR/Cas9-based gene activation system, including the nucleic acid sequence encoding the CRISPR/Cas9-based acetyltransferase and the nucleic acid sequence encoding the at least one gRNA comprising the nucleic acid sequence of at least one of SEQ ID NOs: 23-73, 188-223, or 224-254.

### b) Combinations

The CRISPR/Cas9-based gene activation system composition may be combined with orthogonal dCas9s, TALEs, and zinc finger proteins to facilitate studies of independent targeting of particular effector functions to distinct loci. In some embodiments, the CRISPR/Cas9-based gene activation system composition may be multiplexed with various activators, repressors, and epigenetic modifiers to precisely control cell phenotype or decipher complex networks of gene regulation.

### 5. Methods of Use

Potential applications of the CRISPR/Cas9-based gene activation system are diverse across many areas of science and biotechnology. The CRISPR/Cas9-based gene activation system may be used to activate gene expression of a target gene or target a target enhancer or target regulatory element. The CRISPR/Cas9-based gene activation system may be used to transdifferentiate a cell and/or activate genes related to cell and gene therapy, genetic reprogramming, and regenerative medicine. The CRISPR/Cas9-based gene activation system may be used to reprogram cell lineage specification. Activation of endogenous genes encoding the key regulators of cell fate, rather than forced overexpression of these factors, may potentially lead to more rapid, efficient, stable, or specific methods for genetic reprogramming and transdifferentiation. The CRISPR/Cas9-based gene activation system could provide a greater diversity of transcriptional activators to complement other tools for modulating mammalian gene expression. The CRISPR/Cas9-based gene activation system may be used to compensate for genetic defects, suppress angiogenesis, inactivate oncogenes, activate silenced tumor suppressors, regenerate tissue or reprogram genes.

### 6. Methods of Activating Gene Expression

The present disclosure provides a mechanism for activating the expression of target genes based on targeting a histone acetyltransferase to a target region via a CRISPR/Cas9-based gene activation system, as described above. The CRISPR/Cas9-based gene activation system may activate silenced genes. The CRISPR/Cas9-based gene activation system target regions upstream of the TSS of the target gene and substantially induced gene expression of the target gene. The polynucleotide encoding the CRISPR/Cas9-based gene activation system can also be transfected directly to cells.

The method may include administering to a cell or subject a CRISPR/Cas9-based gene activation system, compositions of CRISPR/Cas9-based gene activation system, or one or more polynucleotides or vectors encoding said CRISPR/Cas9-based gene activation system, as described above. The method may include administering a CRISPR/Cas9-based gene activation system, compositions of CRISPR/Cas9-based gene activation system, or one or more polynucleotides or vectors encoding said CRISPR/Cas9-based gene activation system, as described above, to a mammalian cell or subject.

### 7. Pharmaceutical Compositions

The CRISPR/Cas9-based gene activation system may be in a pharmaceutical composition. The pharmaceutical composition may comprise about 1 ng to about 10 mg of DNA encoding the CRISPR/Cas9-based gene activation system. The pharmaceutical compositions according to the present invention are formulated according to the mode of administration to be used. In cases where pharmaceutical compositions are injectable pharmaceutical compositions, they are sterile, pyrogen free and particulate free. An isotonic formulation is preferably used. Generally, additives for isotonicity may include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

The pharmaceutical composition containing the CRISPR/Cas9-based gene activation system may further comprise a pharmaceutically acceptable excipient. The pharmaceutically acceptable excipient may be functional molecules as vehicles, adjuvants, carriers, or diluents. The pharmaceutically acceptable excipient may be a transfection facilitating agent, which may include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and more preferably, the poly-L-glutamate is present in the pharmaceutical composition containing the CRISPR/Cas9-based gene activation system at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the genetic construct. In some embodiments, the DNA vector encoding the CRISPR/Cas9-based gene activation system may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. Preferably, the transfection facilitating agent is a polyanion, polycation, including poly-L-glutamate (LGS), or lipid.

### 8. Methods of Delivery

Provided herein is a method for delivering the pharmaceutical formulations of the CRISPR/Cas9-based gene activation system for providing genetic constructs and/or proteins of the CRISPR/Cas9-based gene activation system. The delivery of the CRISPR/Cas9-based gene activation system may be the transfection or electroporation of the CRISPR/Cas9-based gene activation system as one or more nucleic acid molecules that is expressed in the cell and delivered to the surface of the cell. The CRISPR/Cas9-based gene activation system protein may be delivered to the cell. The nucleic acid molecules may be electroporated using BioRad Gene Pulser Xcell or Amaxa Nucleofector IIb devices or other electroporation device. Several different buffers may be used, including BioRad electroporation solution, Sigma phosphate-buffered saline product #D8537 (PBS), Invitrogen OptiMEM I (OM), or Amaxa Nucleofector solution V (N.V.). Transfections may include a transfection reagent, such as Lipofectamine 2000.

The vector encoding a CRISPR/Cas9-based gene activation system protein may be delivered to the mammal by DNA injection (also referred to as DNA vaccination) with and without in vivo electroporation, liposome mediated, nanoparticle facilitated, and/or recombinant vectors. The recombinant vector may be delivered by any viral mode. The viral mode may be recombinant lentivirus, recombinant adenovirus, and/or recombinant adeno-associated virus.

The nucleotide encoding a CRISPR/Cas9-based gene activation system protein may be introduced into a cell to induce gene expression of the target gene. For example, one or more nucleotide sequences encoding the CRISPR/Cas9-based gene activation system directed towards a target gene may be introduced into a mammalian cell. Upon delivery of the CRISPR/Cas9-based gene activation system to the cell, and thereupon the vector into the cells of the mammal, the transfected cells will express the CRISPR/Cas9-based gene activation system. The CRISPR/Cas9-based gene activation system may be administered to a mammal to induce or modulate gene expression of the target gene in a mammal. The mammal may be human, non-human primate, cow, pig, sheep, goat, antelope, bison, water buffalo, bovids, deer, hedgehogs, elephants, llama, alpaca, mice, rats, or chicken, and preferably human, cow, pig, or chicken.

### 9. Routes of Administration

The CRISPR/Cas9-based gene activation system and compositions thereof may be administered to a subject by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian may readily determine the dosing regimen and route of administration that is most appropriate for a particular animal. The CRISPR/Cas9-based gene activation system and compositions thereof may be administered by traditional syringes, needleless injection devices, “microprojectile bombardment gone guns”, or other physical methods such as electroporation (“EP”), “hydrodynamic method”, or ultrasound. The composition may be delivered to the mammal by several technologies including DNA injection (also referred to as DNA vaccination) with and without in vivo electroporation, liposome mediated, nanoparticle facilitated, recombinant vectors such as recombinant lentivirus, recombinant adenovirus, and recombinant adenovirus associated virus.

### 10. Cell Types

The CRISPR/Cas9-based gene activation system may be used with any type of cell. In some embodiments, the cell is a bacterial cell, a fungal cell, an archaea cell, a plant cell or an animal cell. In some embodiments, the cell may be an ENCODE cell line, including but not limited to, GM12878, K562, H1 human embryonic stem cells, HeLa-S3, HepG2, HUVEC, SK-N-SH, IMR90, A549, MCF7, HMEC or LHCM, CD14+, CD20+, primary heart or liver cells, differentiated H1 cells, 8988T, Adult_CD4_naive, Adult_CD4_Th0, Adult_CD4_Th1, AG04449, AG04450, AG09309, AG09319, AG10803, AoAF, AoSMC, BC_Adipose_UHN00001, BC_Adrenal_Gland_H12803N, BC_Bladder_01-11002, BC_Brain_H11058N, BC_Breast_02-03015, BC_Colon 01-11002, BC_Colon_H12817N, BC_Esophagus_01-11002, BC_Esophagus_H12817N, BC_Jejunum_H12817N, BC_Kidney_01-11002, BC_Kidney_H12817N, BC_Left_Ventricle_N41, BC_Leukocyte_UHN00204, BC_Liver_01-11002, BC_Lung_01-11002, BC_Lung_H12817N, BC_Pancreas_H12817N, BC_Penis_H12817N, BC_Pericardium_H12529N, BC_Placenta_UHN00189, BC_Prostate_Gland_H12817N, BC_Rectum_N29, BC_Skeletal_Muscle_01-11002, BC_Skeletal_Muscle_H12817N, BC_Skin_01-11002, BC_Small_Intestine_01-11002, BC_Spleen_H12817N, BC_Stomach_01-11002, BC_Stomach_H12817N, BC_Testis_N30, BC_Uterus_BN0765, BE2_C, BG02ES, BG02ES-EBD, BJ, bone_marrow_HS27a, bone_marrow_HS5, bone_marrow_MSC, Breast_OC, Caco-2, CD20+_RO01778, CD20+_RO01794, CD34+_Mobilized, CD4+_Naive_Wb11970640, CD4+_Naive_Wb78495824, Cerebellum_OC, Cerebrum_frontal_OC, Chorion, CLL, CMK, Colo829, Colon_BC, Colon_OC, Cord_CD4_naive, Cord_CD4_Th0, Cord_CD4_Th1, Decidua, Dnd41, ECC-1, Endometrium_OC, Esophagus_BC, Fibrobl, Fibrobl_GM03348, FibroP, FibroP_AG08395, FibroP_AG08396, FibroP_AG20443, Frontal_cortex_OC, GC_B_cell, Gliobla, GM04503, GM04504, GM06990, GM08714, GM10248, GM10266, GM10847, GM12801, GM12812, GM12813, GM12864, GM12865, GM12866, GM12867, GM12868, GM12869, GM12870, GM12871, GM12872, GM12873, GM12874, GM12875, GM12878-XiMat, GM12891, GM12892, GM13976, GM13977, GM15510, GM18505, GM18507, GM18526, GM18951, GM19099, GM19193, GM19238, GM19239, GM19240, GM20000, H0287, H1-neurons, H7-hESC, H9ES, H9ES-AFP−, H9ES-AFP+, H9ES-CM, H9ES-E, H9ES-EB, H9ES-EBD, HAc, HAEpiC, HA-h, HAL, HAoAF, HAoAF_6090101.11, HAoAF_6111301.9, HAoEC, HAoEC_7071706.1, HAoEC_8061102.1, HA-sp, HBMEC, HBVP, HBVSMC, HCF, HCFaa, HCH, HCH_0011308.2P, HCH_8100808.2, HCM, HConF, HCPEpiC, HCT-116, Heart_OC, Heart_STL003, HEEpiC, HEK293, HEK293T, HEK293-T-REx, Hepatocytes, HFDPC, HFDPC_0100503.2, HFDPC_0102703.3, HFF, HFF-Myc, HFL11W, HFL24W, HGF, HHSEC, HIPEpiC, HL-60, HMEpC, HMEpC_6022801.3, HMF, hMNC-CB, hMNC-CB_8072802.6, hMNC-CB_9111701.6, hMNC-PB, hMNC-PB_0022330.9, hMNC-PB_0082430.9, hMSC-AT, hMSC-AT_0102604.12, hMSC-AT_9061601.12, hMSC-BM, hMSC-BM_0050602.11, hMSC-BM_0051105.11, hMSC-UC, hMSC-UC_0052501.7, hMSC-UC_0081101.7, HMVEC-dAd, HMVEC-dBI-Ad, HMVEC-dBl-Neo, HMVEC-dLy-Ad, HMVEC-dLy-Neo, HMVEC-dNeo, HMVEC-LBI, HMVEC-LLy, HNPCEpiC, HOB, HOB_0090202.1, HOB_0091301, HPAEC, HPAEpiC, HPAF, HPC-PL, HPC-PL_0032601.13, HPC-PL_0101504.13, HPDE6-E6E7, HPdLF, HPF, HPIEpC, HPIEpC_9012801.2, HPIEpC_9041503.2, HRCEpiC, HRE, HRGEC, HRPEpiC, HSaVEC, HSaVEC_0022202.16, HSaVEC_9100101.15, HSMM, HSMM_emb, HSMM_FSHD, HSMMtube, HSMMtube_emb, HSMMtube_FSHD, HT-1080, HTR8svn, Huh-7, Huh-7.5, HVMF, HVMF_6091203.3, HVMF_6100401.3, HWP, HWP_0092205, HWP_8120201.5, iPS, iPS_CWRU1, iPS_hFib2_iPS4, iPS_hFib2_iPS5, iPS_NIHil 1, iPS_NIHi7, Ishikawa, Jurkat, Kidney BC, Kidney_OC, LHCN-M2, LHSR, Liver_OC, Liver_STL004, Liver_STLO11, LNCaP, Loucy, Lung_BC, Lung_OC, Lymphoblastoid_cell_line, M059J, MCF10A-Er-Src, MCF-7, MDA-MB-231, Medullo, Medullo_D341, Mel 2183, Melano, Monocytes-CD14+, Monocytes-CD14+_RO01746, Monocytes-CD14+_RO01826, MRT_A204, MRT_G401, MRT_TTC549, Myometr, Naive_B_cell, NB4, NH-A, NHBE, NHBE_RA, NHDF, NHDF_0060801.3, NHDF_7071701.2, NHDF-Ad, NHDF-neo, NHEK, NHEM.f_M2, NHEM.f_M2_5071302.2, NHEM.f_M2_6022001, NHEM_M2, NHEM_M2_7011001.2, NHEM_M2_7012303, NHLF, NT2-D1, Olf_neurosphere, Osteobl, ovcar-3, PANC-1, Pancreas_OC, PanIsletD, PanIslets, PBDE, PBDEFetal, PBMC, PFSK-1, pHTE, Pons_OC, PrEC, ProgFib, Prostate, Prostate_OC, Psoas_muscle_OC, Raji, RCC_7860, RPMI-7951, RPTEC, RWPE1, SAEC, SH-SY5Y, Skeletal_Muscle_BC, SkMC, SKMC, SkMC_8121902.17, SkMC_9011302, SK-N-MC, SK-N-SH_RA, Small_intestine_OC, Spleen_OC, Stellate, Stomach_BC, T_cells_CD4+, T-47D, T98G, TBEC, Th1, Th1_Wb33676984, Th1_Wb54553204, Th17, Th2, Th2_Wb33676984, Th2_Wb54553204, Treg_Wb78495824, Treg_Wb83319432, U2OS, U87, UCH-1, Urothelia, WERI-Rb-1, and WI-38.

### 11. Kits

Provided herein is a kit, which may be used to activate gene expression of a target gene. The kit comprises a composition for activating gene expression, as described above, and instructions for using said composition. Instructions included in kits may be affixed to packaging material or may be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

The composition for activating gene expression may include a modified AAV vector and a nucleotide sequence encoding a CRISPR/Cas9-based gene activation system, as described above. The CRISPR/Cas9-based gene activation system may include CRISPR/Cas9-based acetyltransferase, as described above, that specifically binds and targets a cis-regulatory region or trans-regulatory region of a target gene. The CRISPR/Cas9-based acetyltransferase, as described above, may be included in the kit to specifically bind and target a particular regulatory region of the target gene.

### 12. Examples

The foregoing may be better understood by reference to the following examples, which are presented for purposes of illustration and are not intended to limit the scope of the invention.

### Example 1

### Methods and Materials—Activator

Cell lines and transfection. HEK293T cells were procured from the American Tissue Collection Center (ATCC, Manassas VA) through the Duke University Cell Culture Facility. Cells were cultured in Dulbecco's modified Eagle's medium (DMEM) supplemented with 10% FBS and 1% penicillin/streptomycin and maintained at 37° C. and 5% CO₂. Transfections were performed in 24-well plates using 375 ng of respective dCas9 expression vector and 125 ng of equimolar pooled or individual gRNA expression vectors mixed with Lipofectamine 2000 (Life Technologies, cat. #11668019) as per manufacturer's instruction. For ChIP-qPCR experiments, HEK293T cells were transfected in 15 cm dishes with Lipofectamine 2000 and 30 μg of respective dCas9 expression vector and 10 μg of equimolar pooled gRNA expression vectors as per manufacturer's instruction.

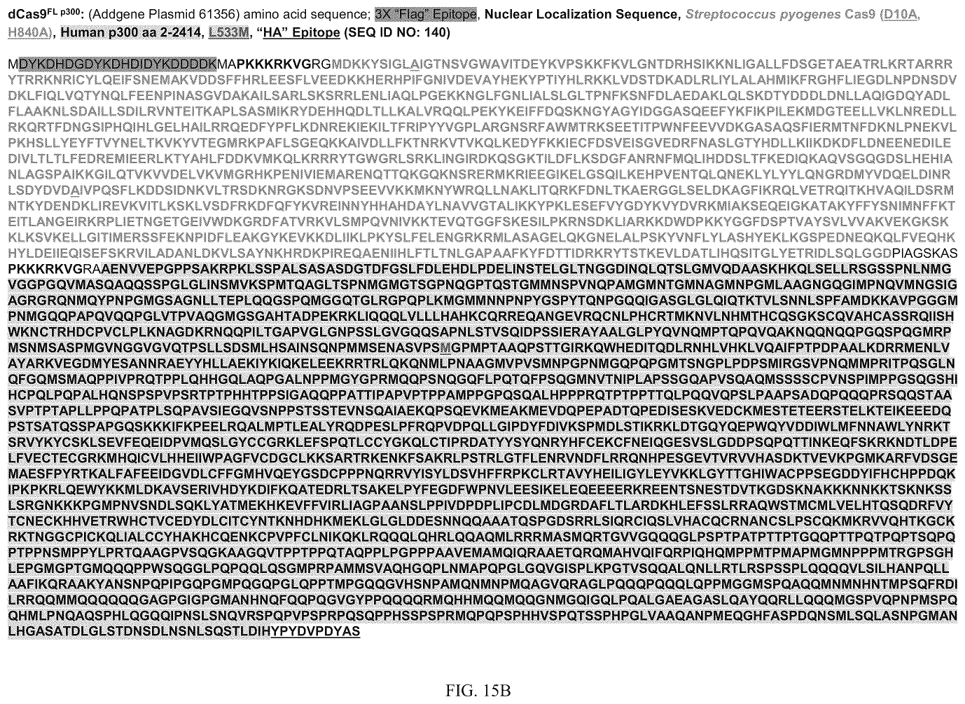

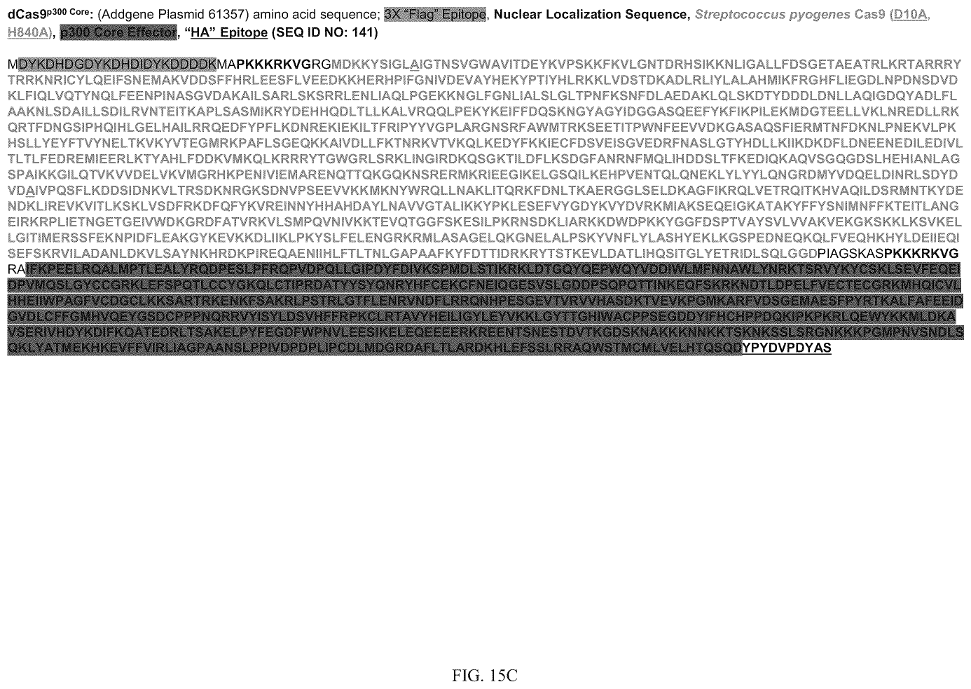

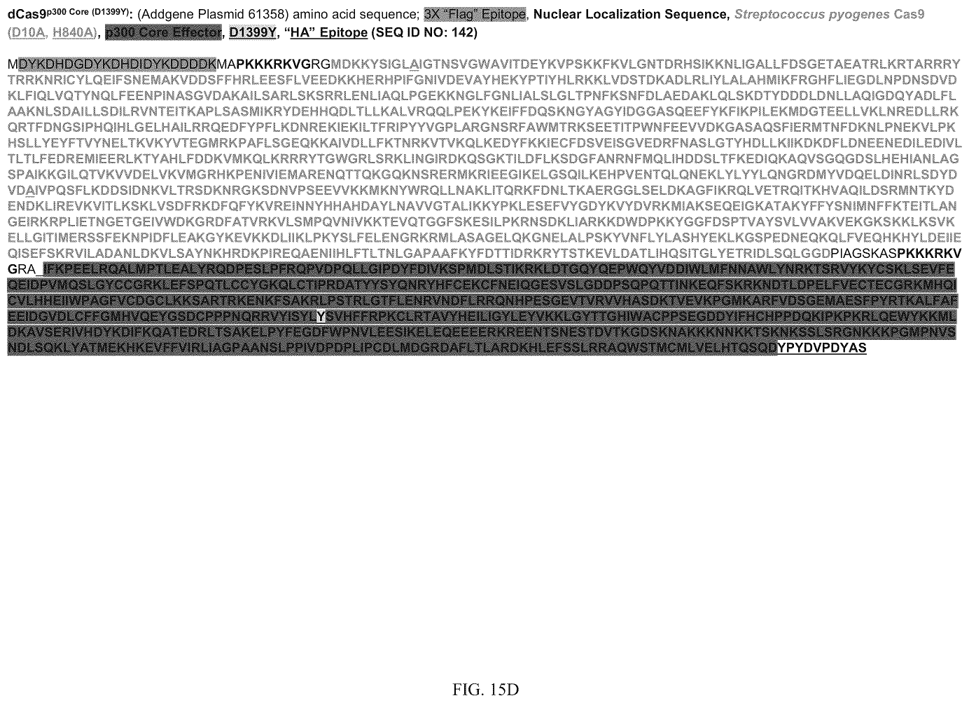

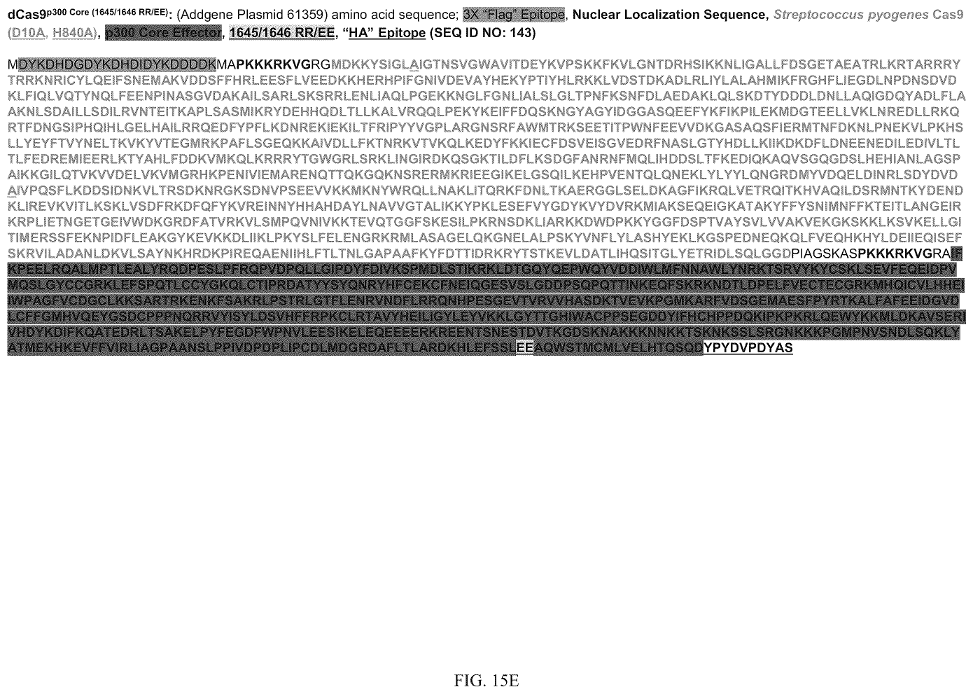

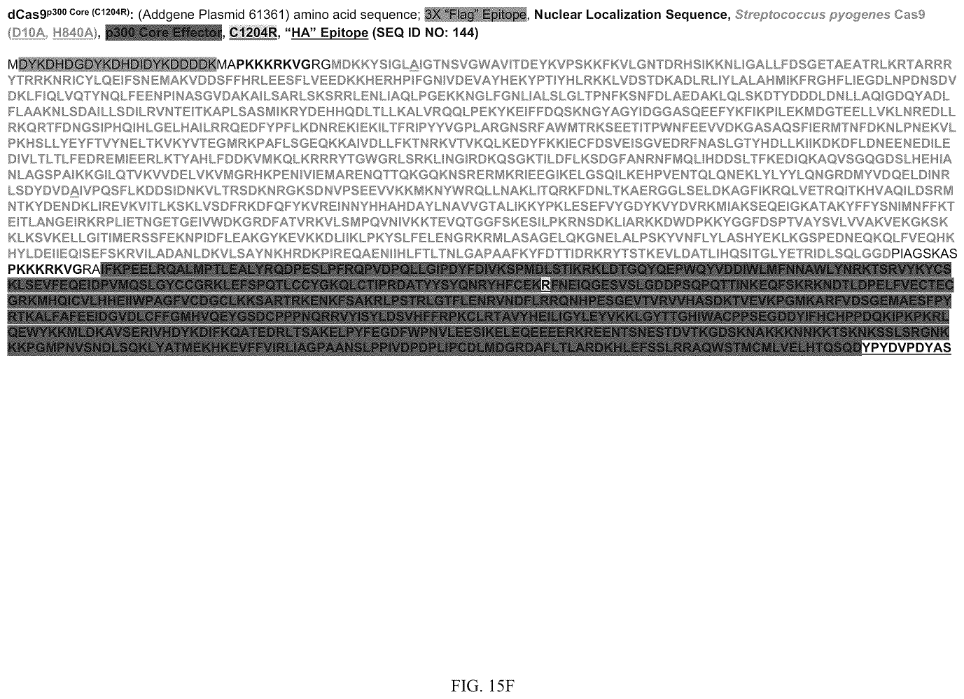

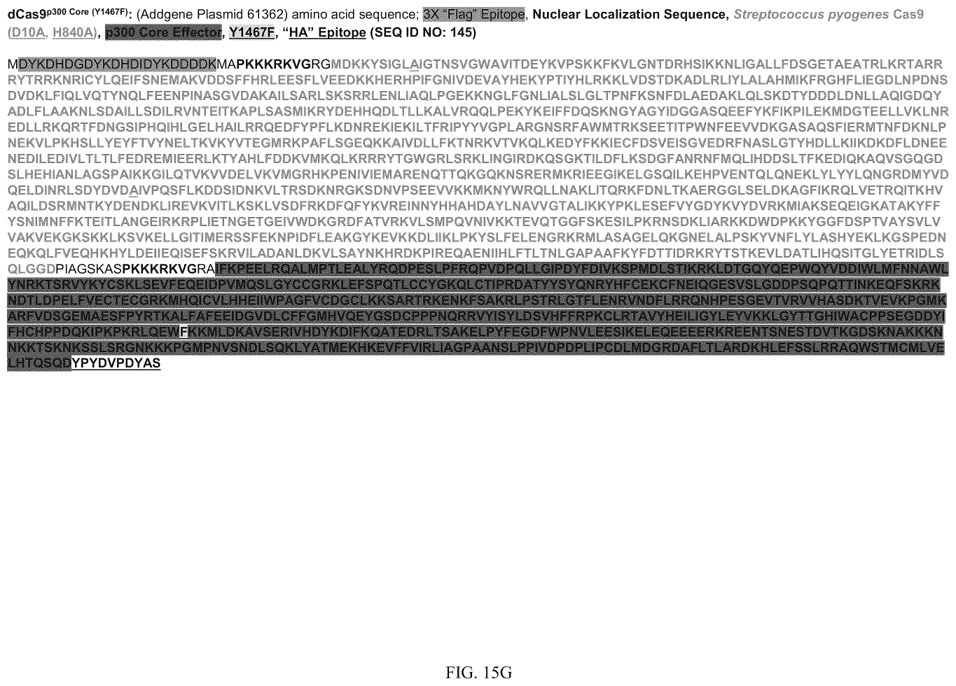

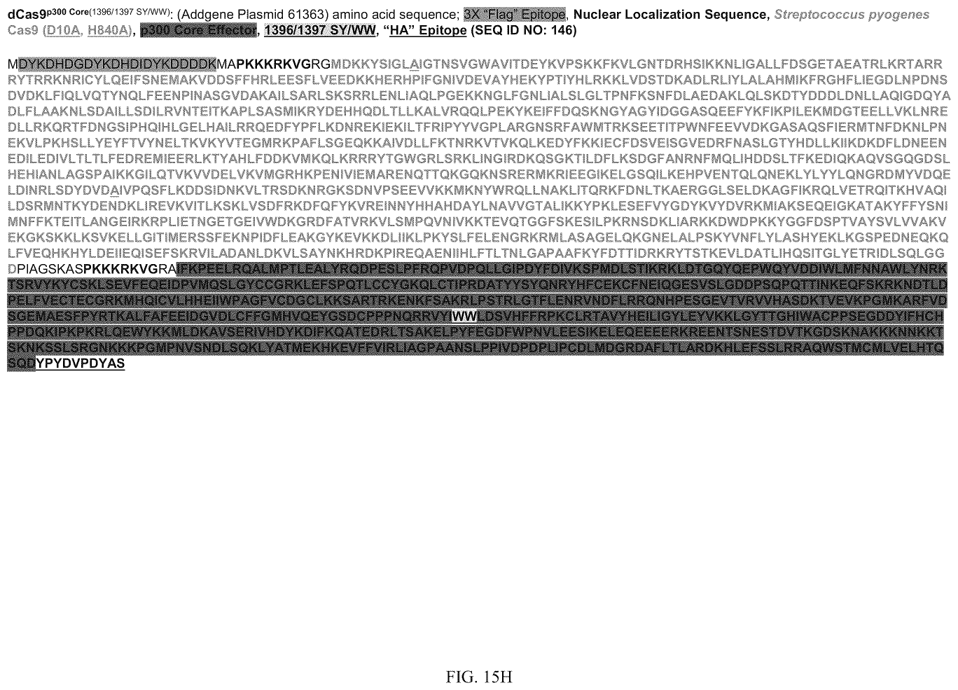

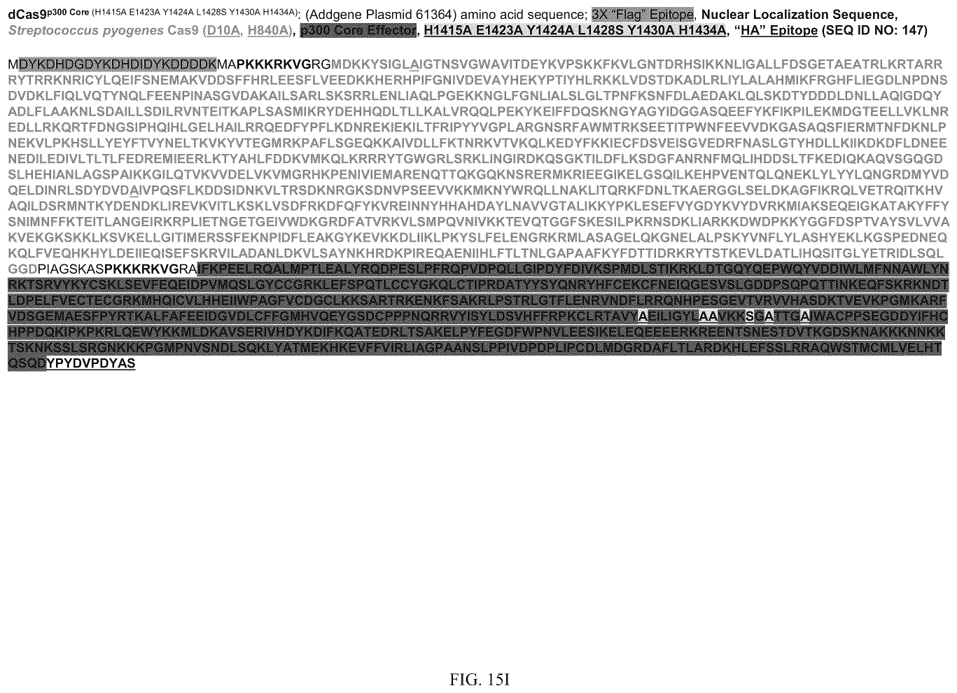

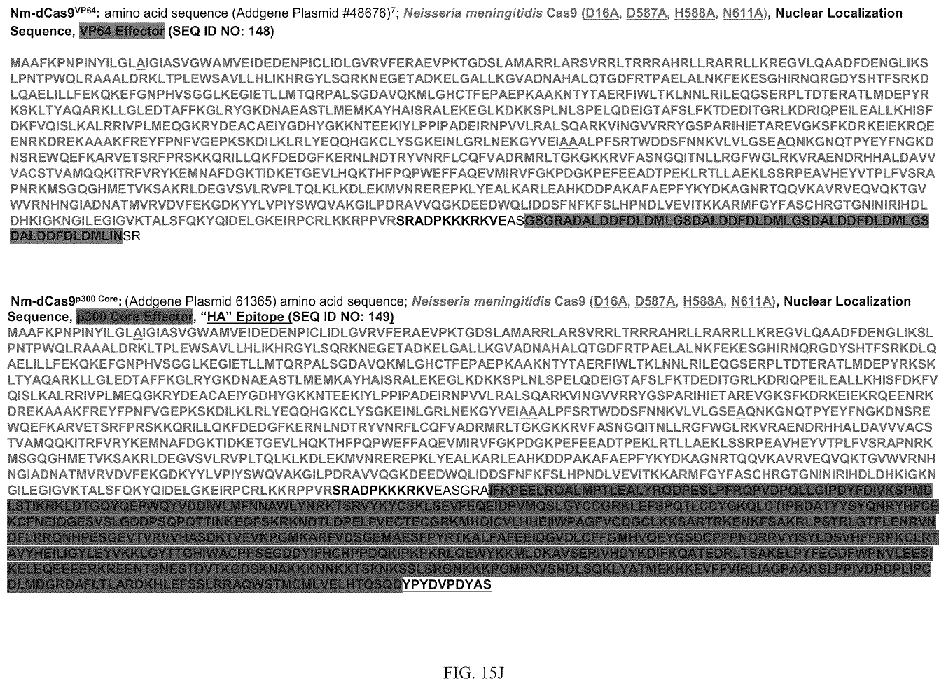

Plasmid constructs. pcDNA-dCas9^{VP64} (dCas9^{VP64}; Addgene, plasmid #47107) was used (Perez-Pinera, P. et al, Nature methods 10:973-976 (2013)). An HA epitope tag was added to dCas9 (no effector) by removing the VP64 effector domain from dCas9^{VP64} via AscI/PacI restriction sites and using isothermal assembly (Gibson et al. Nat. Methods 6:343-345 (2009)) to include an annealed set of oligos containing the appropriate sequence as per manufacturers instruction (NEB cat. #2611). pcDNA-dCas9^{FLp300} (dCas9^{FLp300}) was created by amplifying full-length p300 from pcDNA3.1-p300 (Addgene, plasmid #23252) (Chen et al. EMBO J. 21:6539-6548 (2002)) in two separate fragments and cloning these fragments into the dCas9^{VP64} backbone via isothermal assembly. A substitution in the full-length p300 protein (L553M), located outside of the HAT Core region, was identified in dCas9^{FLp300} and in the precursor pcDNA3.1-p300 during sequence validation. pcDNA-dCas9^{p300 Core} (dCas9^{p300 Core}) was generated by first amplifying amino acids 1048-1664 of human p300 from cDNA and then subcloning the resulting amplicon into pCR-Blunt (pCR-Blunt^{p300 Core}) (Life Technologies cat. #K2700). An AscI site, HA-epitope tag, and a PmeI site were added by PCR amplification of the p300 Core from pCR-Blunt^{p300 Core} and subsequently this amplicon was cloned into pCR-Blunt (pCR-Blunt^{p300 Core+HA}) (Life Technologies cat. #K2700). The HA-tagged p300 Core was cloned from pCR-Blunt^{p300 Core+HA} into the dCas9^{VP64} backbone via shared AscI/PmeI restriction sites. pcDNA-dCas9^{p300 Core (D1399Y)} (dCas9^{p300 Core (D1399Y)}) was generated by amplification of the p300 Core from dCas9^{p300 Core} in overlapping fragments with primer sets including the specified nucleic acid mutations, with a subsequent round of linkage PCR and cloning into the dCas9^{p300 Core} backbone using shared AscI/PmeI restriction sites. All PCR amplifications were carried out using Q5 high-fidelity DNA polymerase (NEB cat. #M0491). Protein sequences of all dCas9 constructs are shown in FIGS. 15A-15J.

IL1RN, MYOD, and OCT4 promoter gRNA protospacers have been described previously (Perez-Pinera, P. et al, Nature methods 10:973-976 (2013); Hu, J. et al., Nucleic Acids Res 42:4375-4390 (2014)). Neisseria meningitidis dCas9^{VP64} (Nm-dCas9^{VP64}) was obtained from Addgene (plasmid #48676). Nm-dCas9^{p300 Core} was created by amplifying the HA-tagged p300 Core from dCas9^{p300 Core} with primers to facilitate subcloning into the AleI/AgeI-digested Nm-dCas9^{VP64} backbone using isothermal assembly (NEB cat. #2611). IL1RN TALE^{p300 Core} TALEs were generated by subcloning the HA-tagged p300 Core domain from dCas9^{p300 Core} into previously published (Perez-Pinera, P. et al, Nature methods 10:973-976 (2013)) IL1RN TALE^{VP64} constructs via shared AscI/PmeI restriction sites. IL1RN TALE target sites are shown in Table 1.

TABLE 1
IL1RN TAL effector information.

| | | SEQ | Location (GRCh37/hg19 |
|---|---|---|---|
| Name | Target Site | ID NO | assembly) |
| IL1RN TALE^{VP64 A} | GGGCTCCTCCTTGTACT | 15 | chr2: 113875431-113875447 |
| IL1RN TALE^{VP64 B} | ACGCAGATAAGAACCAGT | 16 | chr2: 113875291-113875308 |
| IL1RN TALE^{VP64 C} | GGCATCAAGTCAGCCAT | 17 | chr2: 113875356-113875372 |
| IL1RN TALE^{VP64 D} | AGCCTGAGTCACCCTCCT | 18 | chr2: 113875321-113875338 |
| IL1RN TALE^{p300 Core A} | GGGCTCCTCCTTGTACT | 19 | chr2: 113875431-113875447 |
| IL1RN TALE^{p300 Core B} | ACGCAGATAAGAACCAGT | 20 | chr2: 113875291-113875308 |
| IL1RN TALE^{p300 Core C} | GGCATCAAGTCAGCCAT | 21 | chr2: 113875356-113875372 |
| IL1RN TALE^{p300 Core D} | AGCCTGAGTCACCCTCCT | 22 | chr2: 113875321-113875338 |

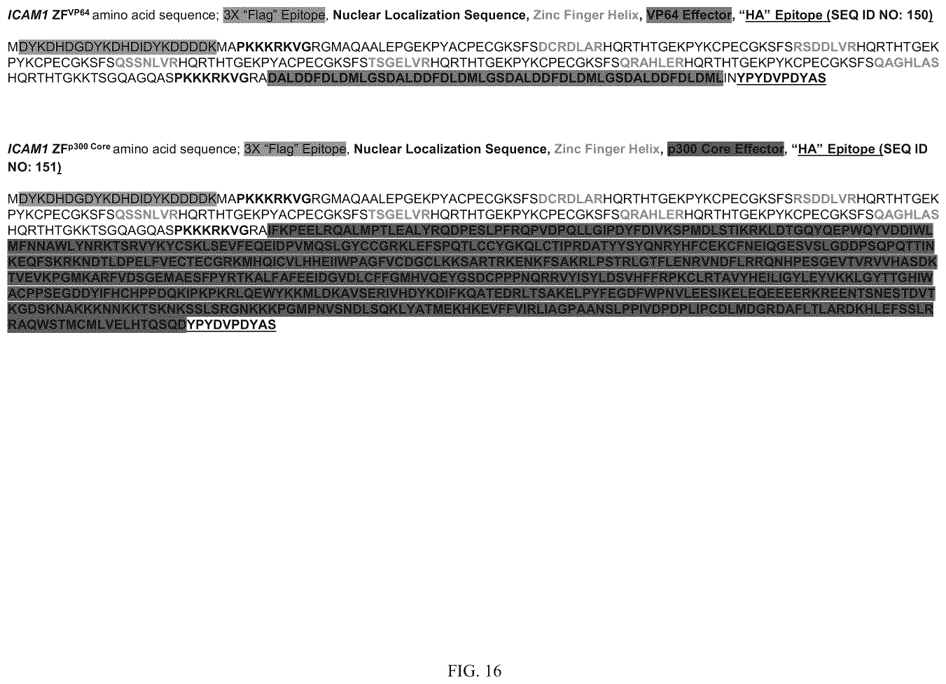

ICAM1 ZF^{VP64} and ICAM1 ZF^{p300 Core} were constructed by subcloning the ICAM1 ZF from pMX-CD54-31Opt-VP64⁵⁴ into dCas9^{VP64} and dCas9^{p300 Core} backbones, respectively, using isothermal assembly (NEB cat. #2611). Protein sequences of ICAM1 ZF constructs are shown in FIG. 16. Transfection efficiency was routinely above 90% as assayed by co-transfection of PL-SIN-EF1α-EGFP (Addgene plasmid #21320) and gRNA empty vector in all experiments. All Streptococcus pyogenes gRNAs were annealed and cloned into pZdonor-pSPgRNA (Addgene plasmid #47108) for expression (Cong, L. et al., Science 339:819-823 (2013)) with slight modifications using NEB BbsI and T4 ligase (Cat. #s R0539 and M0202). Nm-dCas9 gRNA oligos were rationally designed using published PAM requirements (Esvelt, K. M. et al., Nature Methods 10:1116-1121 (2013)), and then cloned into pZDonor-Nm-Cas9-gRNA-hU6 (Addgene, plasmid #61366) via BbsI sites. Plasmids are available through Addgene (Table 2).

TABLE 2
Referenced plasmids in this study available at Addgene.

| Plasmid Name | Addgene Plasmid # |
|---|---|
| pcDNA-dCas9^{VP64} (SEQ ID NO: 139) | 47107 |
| pcDNA-dCas9-HA (SEQ ID NO: 138) | 61355 |
| pcDNA3.1-p300 | 23252 |
| pcDNA-dCas9^{FLp300} (SEQ ID NO: 140) | 61356 |
| pcDNA-dCas9^{p300 Core} (SEQ ID NO: 141) | 61357 |
| pcDNA-dCas9^{p300 Core (D1399Y)} (SEQ ID NO: 142) | 61358 |
| pcDNA-dCas9^{p300 Core (1645/1646 RR/EE)} (SEQ ID NO: 143) | 61359 |
| pcDNA-dCas9^{p300 Core (C1204R)} (SEQ ID NO: 144) | 61361 |
| pcDNA-dCas9^{p300 Core (Y1467F)} (SEQ ID NO: 145) | 61362 |
| pcDNA-dCas9^{p300 Core (1396/1397 SY/WW)} (SEQ ID NO: 146) | 61363 |
| pcDNA-dCas9^{p300 Core (H1415A/E1423A/Y1424A/L1428S/Y1430A/H1434A)} | 61364 |
| (SEQ ID NO: 147) | |
| pZdonor-pSPgRNA | 47108 |
| pcDNA3.1-300(HAT-) | 23254 |
| pcDNA3.3-Nm-dCas9^{VP64} (SEQ ID NO: 148) | 48676 |
| pcDNA3.3-Nm-dCas9^{p300 Core} (SEQ ID NO: 149) | 61365 |
| pZDonor-NmCas9-gRNA-hU6 | 61366 |
| PL-SIN-EF1α-EGFP | 21320 |

All gRNA protospacer targets are listed in Tables 3 and 4.

TABLE 3
gRNA information.

| | | SEQ | |
|---|---|---|---|
| Target | Protospacer Sequence | ID | Genomic Location |
| Location | (5′-3′) | NO | (GRCh37/hg19 Assembly) |
| IL1RN | TGTACTCTCTGAGGTGCTC | 23 | chr2: 113875442-113875460 |
| Promoter A | | | |
| IL1RN | ACGCAGATAAGAACCAGTT | 24 | chr2: 113875291-113875309 |
| Promoter B | | | |
| IL1RN | CATCAAGTCAGCCATCAGC | 25 | chr2: 113875358-113875376 |
| Promoter C | | | |
| IL1RN | GAGTCACCCTCCTGGAAAC | 26 | chr2: 113875326-113875344 |
| Promoter D | | | |
| MYOD | CCTGGGCTCCGGGGCGTTT | 27 | chr11: 17741056-17741074 |
| Promoter A | | | |
| MYOD | GGCCCCTGCGGCCACCCCG | 28 | chr11: 17740969-17740987 |
| Promoter B | | | |
| MYOD | CTCCCTCCCTGCCCGGTAG | 29 | chr11: 17740897-17740915 |
| Promoter C | | | |
| MYOD | AGGTTTGGAAAGGGCGTGC | 30 | chr11: 17740837-17740855 |
| Promoter D | | | |
| OCT4 | ACTCCACTGCACTCCAGTCT | 31 | chr6: 31138711-31138730 |
| Promoter A | | | |
| OCT4 | TCTGTGGGGGACCTGCACTG | 32 | chr6: 31138643-31138662 |
| Promoter B | | | |
| OCT4 | GGGGCGCCAGTTGTGTCTCC | 33 | chr6: 31138613-31138632 |
| Promoter C | | | |
| OCT4 | ACACCATTGCCACCACCATT | 34 | chr6: 31138574-31138593 |
| Promoter D | | | |
| MYOD DRR A | TGTTTTCAGCTTCCAAACT | 35 | chr11: 17736528-17736546 |
| MYOD DRR B | CATGAAGACAGCAGAAGCC | 36 | chr11: 17736311-17736329 |
| MYOD DRR C | GGCCCACATTCCTTTCCAG | 37 | chr11: 17736158-17736176 |
| MYOD DRR D | GGCTGGATTGGGTTTCCAG | 38 | chr11: 17736065-17736083 |
| MYOD CE A | CAACTGAGTCCTGAGGTTT | 39 | chr11: 17721347-17721365 |
| MYOD CE B | CTCACAGCACAGCCAGTGT | 40 | chr11: 17721257-17721275 |
| MYOD CE C | CAGCAGCTGGTCACAAAGC | 41 | chr11: 17721200-17721218 |
| MYOD CE D | CTTCCTATAAACTTCTGAG | 42 | chr11: 17721139-17721157 |
| OCT4 PE A | AGTGATAAGACACCCGCTTT | 43 | chr6: 31139524-31139543 |
| OCT4 PE B | CAGACATCTAATACCACGGT | 44 | chr6: 31139604-31139623 |
| OCT4 PE C | AGGGAGAACGGGGCCTACCG | 45 | chr6: 31139620-31139639 |
| OCT4 PE D | ACTTCAGGTTCAAAGAAGCC | 46 | chr6: 31139725-31139744 |
| OCT4 PE E | TTTTCCCCACCCAGGGCCTA | 47 | chr6: 31139671-31139690 |
| OCT4 PE F | CCCTGGGTGGGGAAAACCAG | 48 | chr6: 31139675-31139694 |
| OCT4 DE A | GGAGGAACATGCTTCGGAAC | 49 | chr6: 31140809-31140828 |
| OCT4 DE B | GTGCCGTGATGGTTCTGTCC | 50 | chr6: 31140864-31140883 |
| OCT4 DE C | GGTCTGCCGGAAGGTCTACA | 51 | chr6: 31140707-31140726 |
| OCT4 DE D | TCGGCCTTTAACTGCCCAAA | 52 | chr6: 31140757-31140776 |
| OCT4 DE E | GCATGACAAAGGTGCCGTGA | 53 | chr6: 31140875-31140894 |
| OCT4 DE F | CCTGCCTTTTGGGCAGTTAA | 54 | chr6: 31140764-31140783 |
| HS2 A | AATATGTCACATTCTGTCTC | 55 | chr11: 5301800-5301819 |
| HS2 B | GGACTATGGGAGGTCACTAA | 56 | chr11: 5302108-5302127 |
| HS2 C | GAAGGTTACACAGAACCAGA | 57 | chr11: 5302033-5302052 |
| HS2 D | GCCCTGTAAGCATCCTGCTG | 58 | chr11: 5301898-5301917 |

TABLE 4

| | | | Genomic Location |
|---|---|---|---|
| | Protospacer Sequence | SEQ | (GRCh37/hg19 |
| Target Location | (5′-3′) | ID NO | Assembly) |
| HBG Promoter A | CCACTGCTAACTGAAAGAGA | 59 | chr11: 5271570-5271589 |
| HBG Promoter B | AGCCACAGTTTCAGCGCAGT | 60 | chr11: 5271692-5271711 |
| HBG Promoter C | CTGTTTCATCTTAGAAAAAT | 61 | chr11: 5271793-5271812 |
| HBG Promoter D | GAATGTTCTTTGGCAGGTAC | 62 | chr11: 5271942-5271961 |
| HBG Promoter E | CGCACATCTTATGTCTTAGA | 63 | chr11: 5272021-5272040 |
| HBE Promoter A | CTTAAGAGAGCTAGAACTGG | 64 | chr11: 5291618-5291637 |
| HBE Promoter B | TCCCAAAGTACAGTACCTTG | 65 | chr11: 5291758-5291777 |
| HBE Promoter C | TCCCTAGAGAGGACAGACAG | 66 | chr11: 5291785-5291804 |
| HBE Promoter D | TCATAGAGAAATGAAAAGAG | 67 | chr11: 5291840-5291859 |
| HBE Promoter E | ATAATATACCCTGACTCCTA | 68 | chr11: 5292038-5292057 |
| HS2 A | AGGCCACCTGCAAGATAAAT | 69 | chr11: 5301662-5301681 |
| HS2 B | TGTTGTTATCAATTGCCATA | 70 | chr11: 5301708-5301727 |
| HS2 C | ATCCCTTCCAGCATCCTCAT | 71 | chr11: 5302187-5302206 |
| HS2 D | GTGCTTCAAAACCATTTGCT | 72 | chr11: 5302245-5302264 |
| HS2 E | GATACATGTTTTATTCTTAT | 73 | chr11: 5302306-5302325 |

Western Blotting. 20 μg of protein was loaded for SDS PAGE and transferred onto a nitrocellulose membrane for western blots. Primary antibodies (as-FLAG; Sigma-Aldrich cat. #F7425 and α-GAPDH; Cell Signaling Technology cat. #14C10) were used at a 1:1000 dilution in TBST+500 Milk. Secondary α-Rabbit HRP (Sigma-Aldrich cat. #A6154) was used at a 1:5000 dilution in TBST+500 Milk. Membranes were exposed after addition of ECL (Bio-Rad cat. #170-5060).

Quantitative reverse-transcription PCR. RNA was isolated from transfected cells using the RNeasy Plus mini kit (Qiagen cat. #74136) and 500 ng of purified RNA was used as template for cDNA synthesis (Life Technologies, cat. #11754). Real-time PCR was performed using PerfeCTa SYBR Green FastMix (Quanta Biosciences, cat. #95072) and a CFX96 Real-Time PCR Detection System with a C1000 Thermal Cycler (Bio-Rad). Baselines were subtracted using the baseline subtraction curve fit analysis mode and thresholds were automatically calculated using the Bio-Rad CFX Manager software version 2.1. Results are expressed as fold change above control mock transfected cells (No DNA) after normalization to GAPDH expression using the ΔΔCt method (Schmittgen et al., Nat. Protoc. 3:1101-1108 (2008)). All qPCR primers and conditions are listed in Table 5.

TABLE 5
Quantitative reverse transcription PCR and ChIP-qPCR primers and
conditions.

| | | SEQ | | SEQ | | |
|---|---|---|---|---|---|---|
| Target | Primer (5′-3′) | ID NO | Reverse Primer (5′-3′) | ID NO | Cycling Parameters | |
| GAPDH | CAATGACCCCTTCATT | 74 | TTGATTTTGGAGGGA | 75 | 95° C. 30 sec | 45X |
| | GACC | | TCTCG | | 95° C. 5 sec | |
| | | | | | 53° C. 20 sec | |
| IL1RN | GGAATCCATGGAGGG | 76 | TGTTCTCGCTCAGGTC | 77 | 95° C. 30 sec | 45X |
| | AAGAT | | AGTG | | 95° C. 5 sec | |
| | | | | | 58° C. 20 sec | |
| MYOD | TCCCTCTTTCACGGTC | 78 | AACACCCGACTGCTG | 79 | 95° C. 30 sec | 45X |
| | TCAC | | TATCC | | 95° C. 5 sec | |
| | | | | | 53° C. 20 sec | |
| OCT4 | CGAAAGAGAAAGCGA | 80 | CGTTGTGCATAGTCG | 81 | 95° C. 30 sec | 45X |
| | ACCAGTATCGAGAAC | | CTGCTTGATCGC | | 95° C. 5 sec | |
| | | | | | 53° C. 20 sec | |
| HBB | GCACGTGGATCCTGAG | 82 | ATTGGACAGCAAGAA | 83 | 95° C. 30 sec | 45X |
| | AACT | | AGCGAG | | 95° C. 5 sec | |
| | | | | | 58° C. 20 sec | |
| HBD | GCACGTGGATCCTGAG | 84 | CAGGAAACAGTCCAG | 85 | 95° C. 30 sec | 45X |
| | AACT | | GATCTCA | | 95° C. 5 sec | |
| | | | | | 58° C. 20 sec | |
| HBG | GCTGAGTGAACTGCAC | 86 | GAATTCTTTGCCGAA | 87 | 95° C. 30 sec | 45X |
| | TGTGA | | ATGGA | | 95° C. 5 sec | |
| | | | | | 58° C. 20 sec | |
| HBE | TCACTAGCAAGCTCTC | 88 | AACAACGAGGAGTCT | 89 | 95° C. 30 sec | 45X |
| | AGGC | | GCCC | | 95° C. 5 sec | |
| | | | | | 62° C. 20 sec | |
| ICAM1 | GCAGACAGTGACCATC | 90 | CAATCCCTCTCGTCC | 91 | 95° C. 30 sec | 45X |
| | TACAGCTT | | AGTCG | | 95° C. 5 sec | |
| | | | | | 58° C. 20 sec | |
| HS2 | TGCTTGGACTATGGGA | 92 | GCAGGTGCTTCAAAA | 93 | 95° C. 30 sec | 45X |
| ChIP | GGTC | | CCATT | | 95° C. 5 sec | |
| Region 1 | | | | | 60° C. 20 sec | |
| HS2 | TCAGGTGGTCAGCTTC | 94 | AAGCAAACCTTCTGG | 95 | 95° C. 30 sec | 45X |
| ChIP | TCCT | | CTCAA | | 95° C. 5 sec | |
| Region 2 | | | | | 60° C. 20 sec | |
| HS2 | CCACACAGGTGAACCC | 96 | GGACACATGCTCACA | 97 | 95° C. 30 sec | 45X |
| ChIP | TTTT | | TACGG | | 95° C. 5 sec | |
| Region 3 | | | | | 60° C. 20 sec | |
| HBE | ATTCGATCCATGTGCC | 98 | CAATGCTGGAATTTG | 99 | 95° C. 30 sec | 45X |
| ChIP | TGA | | TGGAA | | 95° C. 5 sec | |
| Region 1 | | | | | 60° C. 20 sec | |
| HBE | GGGGTGATTCCCTAGA | 100 | AAGCAGGACAGACA | 101 | 95° C. 30 sec | 45X |
| ChIP | GAGG | | GGCAAG | | 95° C. 5 sec | |
| Region 2 | | | | | 60° C. 20 sec | |
| HBE | GAGGGTCAGCAGTGA | 102 | TGGAAAAGGAGAATG | 103 | 95° C. 30 sec | 45X |
| ChIP | TGGAT | | GGAGA | | 95° C. 5 sec | |
| Region 3 | | | | | 60° C. 20 sec | |
| HBG1/2 | TGGTCAAGTTTGCCTT | 104 | GGAATGACTGAATCG | 105 | 95° C. 30 sec | 45X |
| ChIP | GTCA | | GAACAA | | 95° C. 5 sec | |
| Region 1 | | | | | 60° C. 20 sec | |
| HBG1/2 | CCTCCAGCATCTTCCA | 106 | GAAGCACCCTTCAGC | 107 | 95° C. 30 sec | 45X |
| ChIP | CATT | | AGTTC | | 95° C. 5 sec | |
| Region 2 | | | | | 60° C. 20 sec | |
| HBG1/2 | CCACAGTTTCAGCGCA | 108 | ATCAGCCAGCACACA | 109 | 95° C. 30 sec | 45X |
| ChIP | GTAATA | | CACTT | | 95° C. 5 sec | |
| Region 3 | | | | | 60° C. 20 sec | |
| IL1RN | CCCTGTCAGGAGGGAC | 110 | GGCTCACCGGAAGCA | 111 | 95° C. 30 sec | 45X |
| ChIP | AGAT | | TGAAT | | 95° C. 5 sec | |
| Region 1 | | | | | 60° C. 20 sec | |
| IL1RN | AAGCTACAAGCAGGTT | 112 | AATAACAGGGTCCAT | 113 | 95° C. 30 sec | 45X |
| ChIP | CGCT | | CCCGC | | 95° C. 5 sec | |
| Region 2 | | | | | 60° C. 20 sec | |
| IL1RN | TGTTCCCTCCACCTGG | 114 | GGGAAAATCCAAAGC | 115 | 95° C. 30 sec | 45X |
| ChIP | AATA | | AGGAT | | 95° C. 5 sec | |
| Region 3 | | | | | 60° C. 20 sec | |
| IL1RN | TCCTAGGTCCCTCAAA | 116 | GTCCCCAACGCTCTA | 117 | 95° C. 30 sec | 45X |
| ChIP | AGCA | | ACAAA | | 95° C. 5 sec | |
| Region 4 | | | | | 60° C. 20 sec | |
| IL1RN | GTTAGAGCGTTGGGGA | 118 | CACATGCAGAGAACT | 119 | 95° C. 30 sec | 45X |
| ChIP | CCTT | | GAGCTG | | 95° C. 5 sec | |
| Region 5 | | | | | 60° C. 20 sec | |
| IL1RN | GTTGGGGTAAGCACG | 120 | TTTCCAGGAGGGTGA | 121 | 95° C. 30 sec | 45X |
| ChIP | AAGG | | CTCAG | | 95° C. 5 sec | |
| Region 6 | | | | | 60° C. 20 sec | |
| IL1RN | TTCTCTGCATGTGACC | 122 | ACACACTCACAGAGG | 123 | 95° C. 30 sec | 45X |
| ChIP | TCCC | | GTTGG | | 95° C. 5 sec | |
| Region 7 | | | | | 60° C. 20 sec | |
| IL1RN | TGAGTCACCCTCCTGG | 124 | CTCCTTCCAGAGCAC | 125 | 95° C. 30 sec | 45X |
| ChIP | AAAC | | CTCAG | | 95° C. 5 sec | |
| Region 8 | | | | | 60° C. 20 sec | |
| IL1RN | GCTGGGCTCCTCCTTG | 126 | GCTGCTGCCCATAAA | 127 | 95° C. 30 sec | 45X |
| ChIP | TACT | | GTAGC | | 95° C. 5 sec | |
| Region 9 | | | | | 60° C. 20 sec | |
| IL1RN | GGACTGTGGCCCAGGT | 128 | GGCCTCATAGGACAG | 129 | 95° C. 30 sec | 45X |
| ChIP | ACT | | GAGGT | | 95° C. 5 sec | |
| Region | | | | | 60° C. 20 sec | |
| 10 | | | | | | |
| IL1RN | TTATGGGCAGCAGCTC | 130 | GACATTTTCCTGGAC | 131 | 95° C. 30 sec | 45X |
| ChIP | AGTT | | GCTTG | | 95° C. 5 sec | |
| Region | | | | | 60° C. 20 sec | |
| 11 | | | | | | |
| IL1RN | CCCTCCCCATGGCTTT | 132 | AGCTCCATGCGCTTG | 133 | 95° C. 30 sec | 45X |
| ChIP | AGGT | | ACATT | | 95° C. 5 sec | |
| Region | | | | | 60° C. 20 sec | |
| 12 | | | | | | |
| IL1RN | AGCGTCCAGGAAAAT | 134 | ATGACCCTCACACTC | 135 | 95° C. 30 sec | 45X |
| ChIP | GTCAA | | CAAGG | | 95° C. 5 sec | |
| Region | | | | | 60° C. 20 sec | |
| 13 | | | | | | |
| Upstream | GTTGGGTGCTCCAGCT | 136 | CCTCAAAACTCCTGG | 137 | 95° C. 30 sec | 45X |
| β-actin | TTTA | | ACTCG | | 95° C. 5 sec | |
| ChIP | | | | | 60° C. 20 sec | |
| NEG | | | | | | |
| CTRL | | | | | | |

RNA-seq. RNA-seq was performed using three replicates per experimental condition. RNA was isolated from transfected cells using the RNeasy Plus mini kit (Qiagen cat. #74136) and 1 μg of purified mRNA was used as template for cDNA synthesis and library construction using the PrepX RNA-Seq Library Kit (Wafergen Biosystems, cat. #400039). Libraries were prepared using the Apollo 324 liquid handling platform, as per manufacturer's instruction. Indexed libraries were validated for quality and size distribution using the Tapestation 2200 (Agilent) and quantified by qPCR using the KAPA Library Quantification Kit (KAPA Biosystems; KK4835) prior to multiplex pooling and sequencing at the Duke University Genome Sequencing Shared Resource facility. Libraries were pooled and then 50 bp single-end reads were sequenced on a Hiseq 2500 (Illumina), de-multiplexed and then aligned to the HG19 transcriptome using Bowtie 2 (Langmead et al. Nat. Methods 9:357-359 (2012)). Transcript abundance was calculated using the SAMtools (Li et al. Bioinformatics 25:2078:2079 (2009)) suite and differential expression was determined in R using the DESeq2 analysis package. Multiple hypothesis correction was performed using the method of Benjamini and Hochberg with a FDR of <5%. RNA-seq data is deposited in the NCBI's Gene Expression Omnibus and is accessible through GEO Series accession number GSE66742.

ChIP-qPCR. HEK293T cells were co-transfected with four HS2 enhancer gRNA constructs and indicated dCas9 fusion expression vectors in 15 cm plates in biological triplicate for each condition tested. Cells were cross-linked with 1% Formaldehyde (final concentration; Sigma F8775-25ML) for 10 min at RT and then the reaction was stopped by the addition of glycine to a final concentration of 125 mM. From each plate ˜2.5e7 cells were used for H3K27ac ChIP-enrichment. Chromatin was sheared to a median fragment size of 250 bp using a Bioruptor XL (Diagenode). H3K27ac enrichment was performed by incubation with 5 μg of Abcam ab4729 and 200 μl of sheep anti-rabbit IgG magnetic beads (Life Technologies 11203D) for 16 hrs at 4° C. Cross-links were reversed via overnight incubation at 65° C. with sodium dodecyl sulfate, and DNA was purified using MinElute DNA purification columns (Qiagen). 10 ng of DNA was used for subsequent qPCR reactions using a CFX96 Real-Time PCR Detection System with a C1000 Thermal Cycler (Bio-Rad). Baselines were subtracted using the baseline subtraction curve fit analysis mode and thresholds were automatically calculated using the Bio-Rad CFX Manager software version 2.1. Results are expressed as fold change above cells co-transfected with dCas9 and four HS2 gRNAs after normalization to β-actin enrichment using the ΔΔCt method (Schmittgen et al., Nat. Protoc. 3:1101-1108 (2008)). All ChIP-qPCR primers and conditions are listed in Table 5.

### Example 2

### A dCas9 fusion to the p300 HAT domain activates target genes

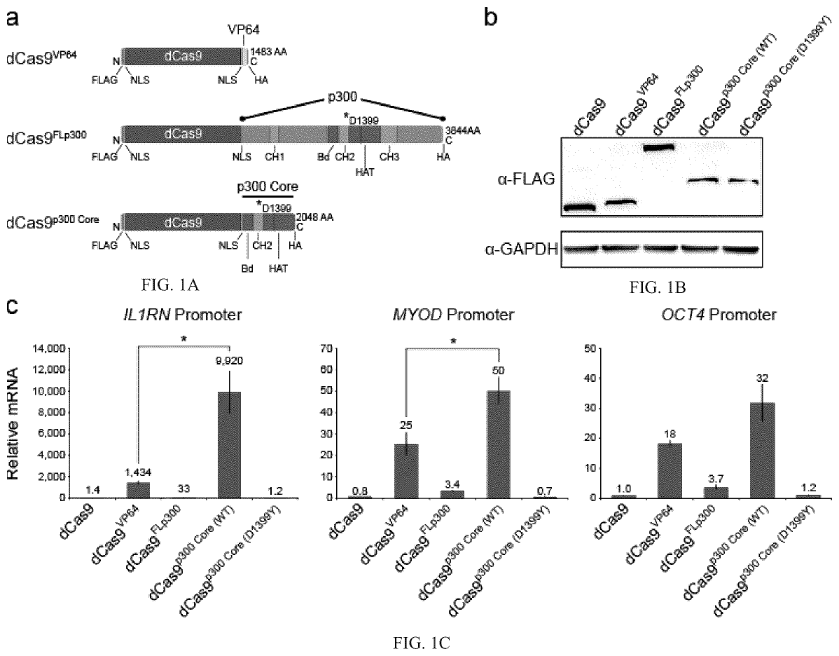

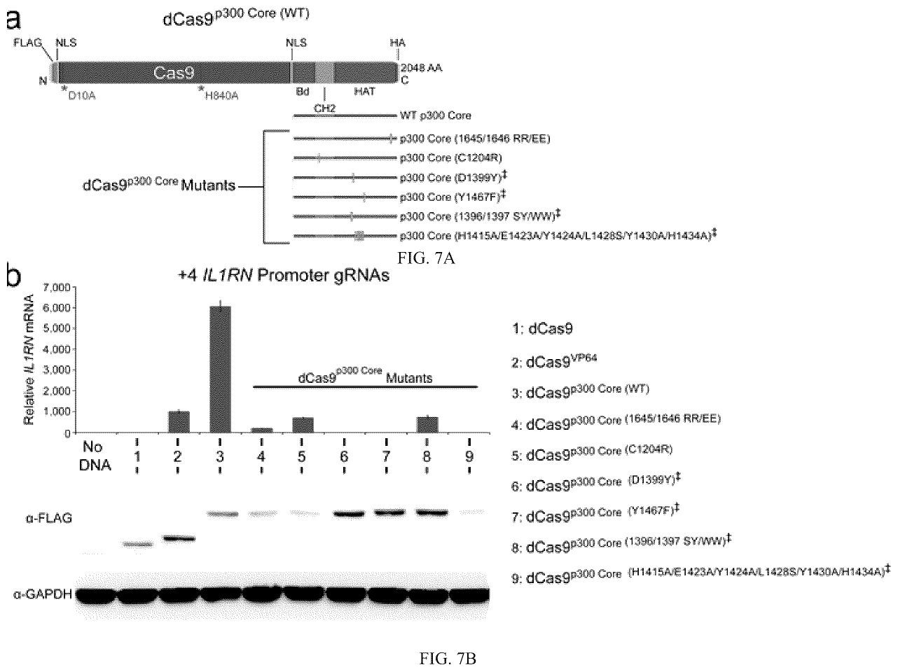

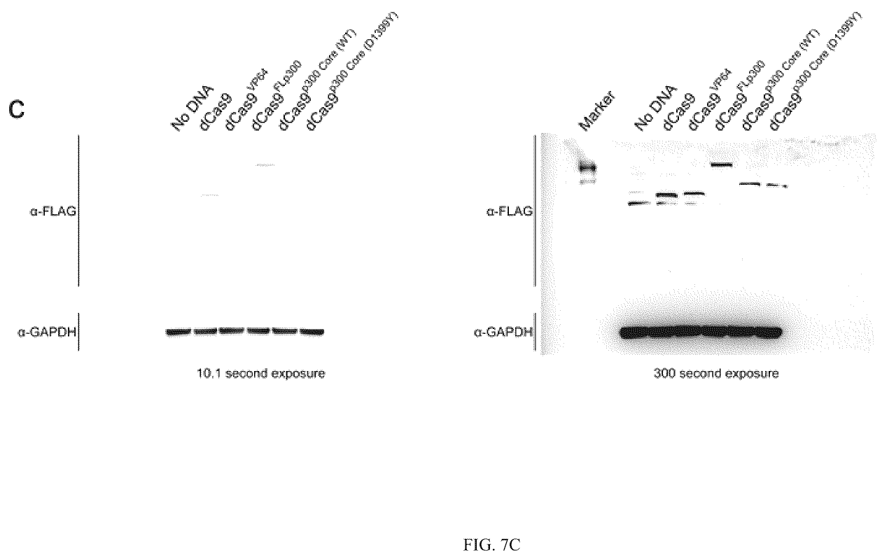

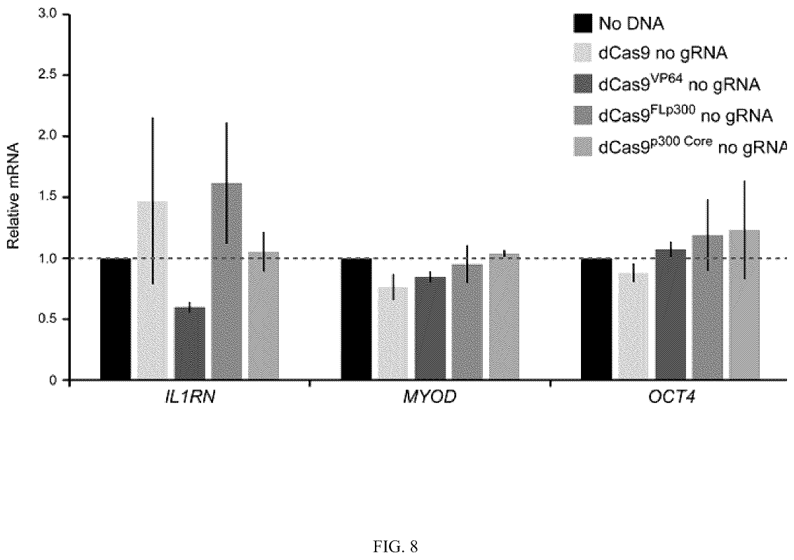

The full-length p300 protein was fused to dCas9 (dCas9^{FLp300}; FIGS. 1A-1i) and assayed for its capacity for transactivation by transient co-transfection of human HEK293T cells with four gRNAs targeting the endogenous promoters of IL1RN, MYOD1 (MYOD), and POU5F1/OCT4 (OCT4) (FIG. 1C). A combination of four gRNAs targeting each promoter was used. dCas9^{FLp300} was well expressed and induced modest activation above background compared to the canonical dCas9 activator fused to the VP64 acidic activation domain (dCas9^{VP64}) (FIGS. 1A-1C). The full-length p300 protein is a promiscuous acetyltransferase which interacts with a multitude of endogenous proteins, largely via its termini. In order to mitigate these interactions the contiguous region of full-length p300 (2414 aa) solely required for inherent HAT activity (amino acids 1048-1664), known as the p300 HAT core domain (p300 Core) was isolated. When fused to the C-terminus of dCas9 (dCas9^{p300 Core}, FIGS. 1A-1) the p300 Core domain induced high levels of transcription from endogenous gRNA-targeted promoters (FIG. 1C). When targeted to the IL1RN and MYOD promoters, the dCas9^{p300 Core} fusion displayed significantly higher levels of transactivation than dCas9^{VP64} (P-value 0.01924 and 0.0324 respectively; FIGS. 1A-1C). These dCas9-effector fusion proteins were expressed at similar levels (FIG. 1B, FIGS. 7A-7C) indicating that the observed differences were due to differences to transactivation capacity. Additionally, no changes to target gene expression were observed when the effector fusions were transfected without gRNAs (FIG. 8). For FIG. 8, dCas9 fusion proteins were transiently co-transfected with an empty gRNA vector backbone and mRNA expression of IL1RN, MYOD, and OCT4 was assayed as in the main text. Red dashed line indicates background expression level from No DNA-transfected cells. n=2 independent experiments, error bars: s.e.m., no significant activation was observed for any target gene assayed.

To ensure that the p300 Core acetyltransferase activity was responsible for gene transactivation using the dCas9^{p300 Core} fusion, a panel of dCas9^{p300 Core} HAT-domain mutant fusion proteins was screened (FIGS. 7A-7C). A single inactivating amino acid substitution within the HAT core domain (WT residue D1399 of full-length p300) of dCas9^{p300 Core} (dCas9^{p300 Core (D1399Y)} (FIG. 1A) abolished the transactivation capacity of the fusion protein (FIG. 1C), demonstrating that intact p300 Core acetyltransferase activity was required for dCas9^{p300 Core}-mediated transactivation.

### Example 3

### dCas9^{p300 Core} Activates Genes from Proximal and Distal Enhancers

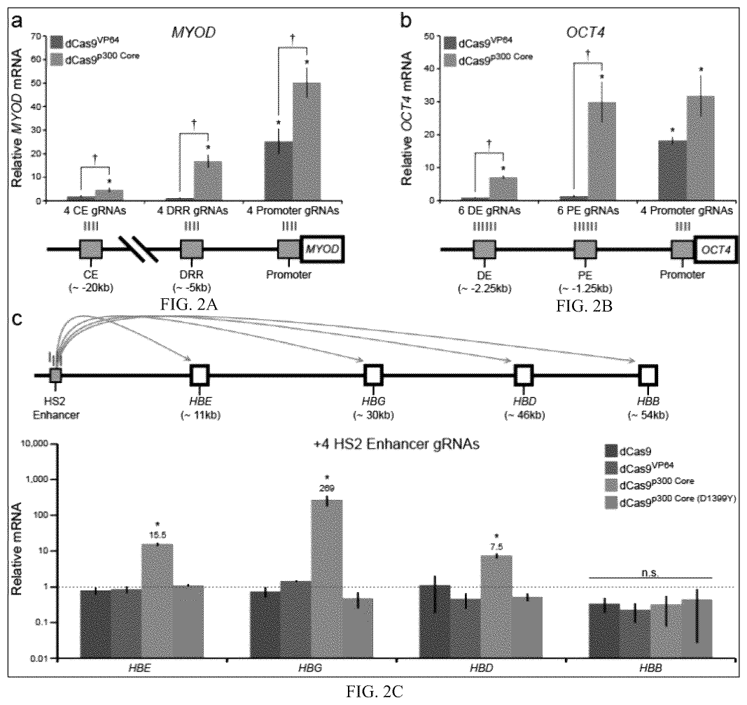

As p300 plays a role and is localized at endogenous enhancers, the dCas9^{p300 Core} may effectively induce transcription from distal regulatory regions with appropriately targeted gRNAs. The distal regulatory region (DRR) and core enhancer (CE) of the human MYOD locus was targeted through co-transfection of four gRNAs targeted to each region and either dCas9^{VP64}or dCas9^{p300 Core} (FIG. 2A). Compared to a mock-transfected control, dCas9^{VP64} did not show any induction when targeted to the MYOD DRR or CE region. In contrast, dCas9^{p300 Core} induced significant transcription when targeted to either MYOD regulatory element with corresponding gRNAs (P-value 0.0115 and 0.0009 for the CE and DRR regions respectively). The upstream proximal (PE) and distal (DE) enhancer regions of the human OCT4 gene were also targeted by co-transfection of six gRNAs and either dCas9^{VP64} or dCas9^{p300 Core} (FIG. 2B). dCas9^{p300 Core} induced significant transcription from these regions (P-value <0.0001 and P-value <0.003 for the DE and PE, respectively), whereas dCas9^{VP64} was unable to activate OCT4 above background levels when targeted to either the PE or DE regions.

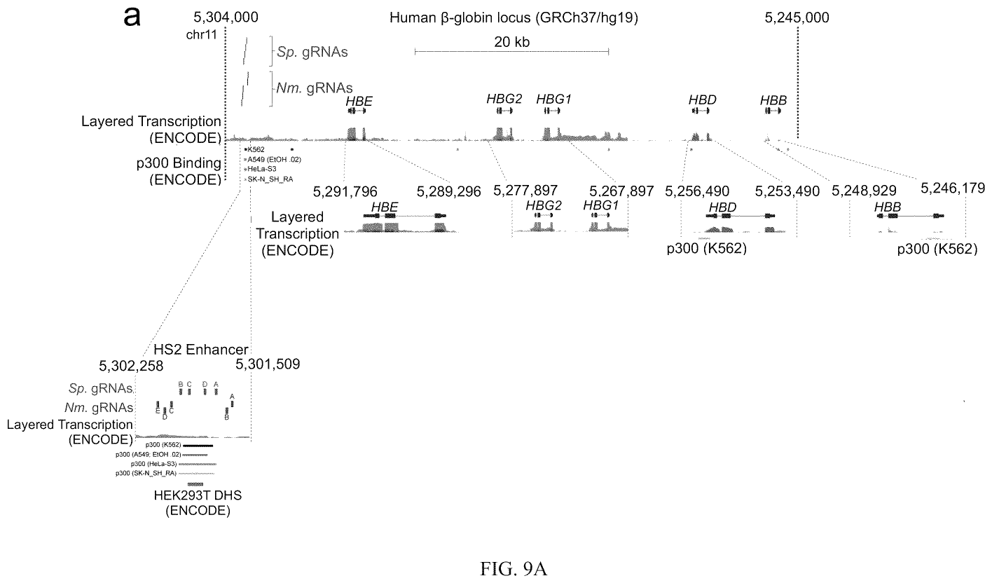

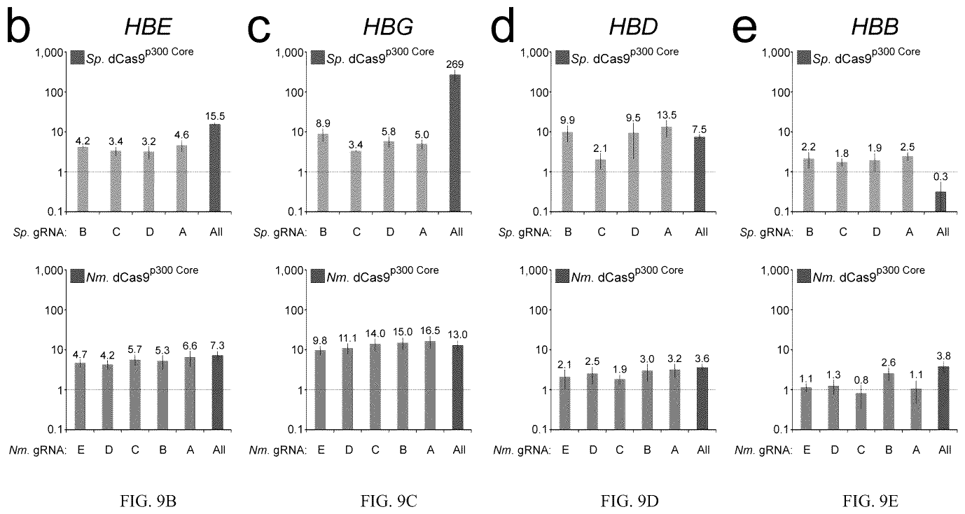

The well-characterized mammalian β-globin locus control region (LCR) orchestrates transcription of the downstream hemoglobin genes; hemoglobin epsilon 1 (HBE, from ˜11 kb), hemoglobin gamma 1 and 2 (HBG, from ˜30 kb), hemoglobin delta (HBD, from ˜46 kb), and hemoglobin beta (HBB, from −54 kb) (FIG. 2C). DNase hypersensitive sites within the β-globin LCR serve as docking sites for transcriptional and chromatin modifiers, including p300, which coordinate distal target gene expression. Four gRNAs targeting the DNase hypersensitive site 2 within the LCR enhancer region (HS2 enhancer) were generated. These four HS2-targeted gRNAs were co-transfected with dCas9, dCas9^{VP64} dCas9^{p300 Core}, or dCas9^{p300 Core (D1399Y)} and the resulting mRNA production from HBE, HBG, HBD, and HBB was assayed (FIG. 2C). dCas9, dCas9^{VP64} and dCas9^{p300 Core (D1399Y)} were unable to transactivate any downstream genes when targeted to the HS2 enhancer. In contrast, targeting of dCas9^{p300 Core} to the HS2 enhancer led to significant expression of the downstream HBE, HBG, and HBD genes (P-value <0.0001, 0.0056, and 0.0003 between dCas9^{p300 Core} and mock-transfected cells for HBE, HBG, and HBD respectively). Overall, HBD and HBE appeared relatively less responsive to synthetic p300 Core-mediated activation from the HS2 enhancer; a finding consistent with lower rates of general transcription from these two genes across several cell lines (FIGS. 9A-9E).

Nevertheless, with the exception of the most distal HBB gene, dCas9^{p300 Core} exhibited a capacity to activate transcription from downstream genes when targeted to all characterized enhancer regions assayed, a capability not observed for dCas9^{VP64} Together, these results demonstrate that dCas9^{p300 Core} is a potent programmable transcription factor that can be used to regulate gene expression from a variety of promoter-proximal and promoter-distal locations.

### Example 4

### Gene Activation by dCas9^{p300 Core} is Highly Specific

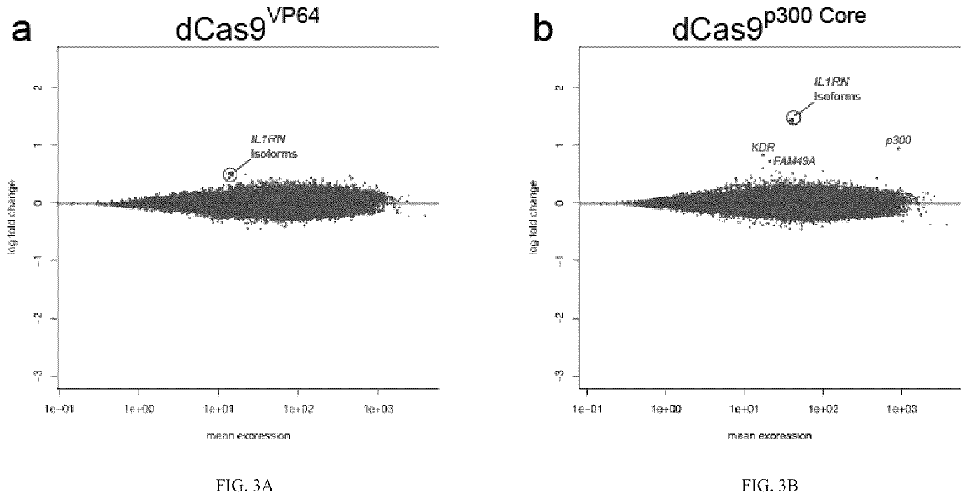

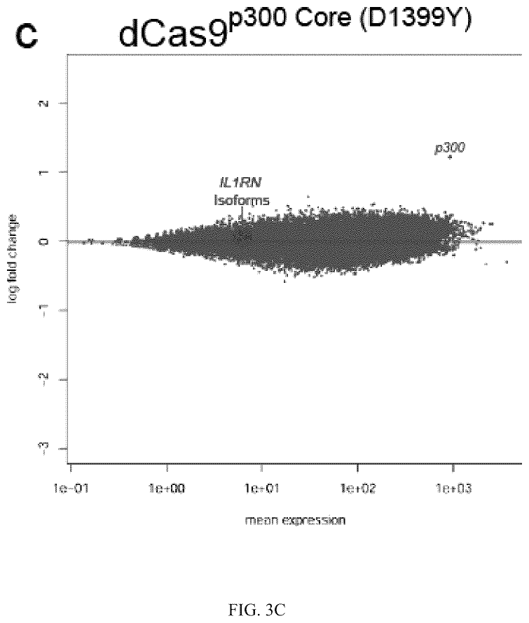

Recent reports indicate that dCas9 may have widespread off-target binding events in mammalian cells in combination with some gRNAs, which could potentially lead to off-target changes in gene expression. In order to assess the transcriptional specificity of the dCas9^{p300 Core} fusion protein, transcriptome was performed profiling by RNA-seq in cells co-transfected with four IIL1RN-targeted gRNAs and either dCas9, dCas9^{VP64}, dCas9^{p300 Core}, or dCas9^{p300 Core (D1399Y)} Genome-wide transcriptional changes were compared between dCas9 with no fused effector domain and either dCas9^{VP64}, dCas9^{p300 Core} or dCas9^{p300} Core (D1399Y) (FIGS. 3A-3C). While both dCas9^{VP64} and dCas9^{p300 Core} upregulated all four IL1RN isoforms, only the effects of dCas9^{p300 Core} reached genome-wide significance (FIGS. 3A-3B, Table 6; P-value 1.0×10⁻³-5.3×10⁻⁴ for dCas9^{VP64}; P-value 1.8×10⁻¹⁷-1.5×10⁻¹⁹ for dCas9^{p300 Core})

TABLE 6
Ten most enriched mRNAs for dCas9 IL1RN-targeted RNA-seq experiments
dCas9^{VP64} + 4 IL1RN gRNAs compared to dCas9 + 4 IL1RN gRNAs

| | | | | log2 Fold | | | | |
|---|---|---|---|---|---|---|---|---|
| | Refseq ID | Gene | Base Mean | Change | lfcSE | stat | pvalue | padj |
| 1 | NM_173842 | IL1RN (transcript variant 1) | 14.764 | 0.529 | 0.152 | 3.48 | 0.000494857 | 0.99992134 |
| 2 | NM_173843 | IL1RN (transcript variant 4) | 13.606 | 0.517 | 0.149 | 3.47 | 0.000530109 | 0.99992134 |
| 3 | NR_073102 | ZNF551 | 21.505 | 0.505 | 0.159 | 3.17 | 0.00152863 | 0.99992134 |
| 4 | NM_000577 | IL1RN (transcript variant 3) | 14.890 | 0.497 | 0.152 | 3.28 | 0.001039353 | 0.99992134 |
| 5 | NM_001077441 | BCLAF1 (transcript variant 3) | 437.814 | 0.482 | 0.153 | 3.14 | 0.001665925 | 0.99992134 |
| 6 | NM_173841 | IL1RN (transcript variant 2) | 13.711 | 0.448 | 0.15 | 3.00 | 0.002716294 | 0.99992134 |
| 7 | NM_001268 | RCBTB2 | 46.265 | 0.440 | 0.167 | 2.64 | 0.008335513 | 0.99992134 |
| 8 | NM_000922 | PDE3B | 143.947 | 0.439 | 0.167 | 2.63 | 0.008471891 | 0.99992134 |
| 9 | NM_001077440 | BCLAF1 (transcript variant 2) | 463.743 | 0.439 | 0.156 | 2.82 | 0.004790762 | 0.99992134 |
| 10 | NM_014739 | BCLAF1 (transcript variant 1) | 474.598 | 0.432 | 0.158 | 2.74 | 0.006232218 | 0.99992134 |

dCas9^{p300 Core} + 4 IL1RN gRNAs compared to dCas9 + 4 IL1RN gRNAs

| | | | | log2 Fold | | | | |
|---|---|---|---|---|---|---|---|---|
| | Refseq ID | Gene | Base Mean | Change | lfcSE | stat | pvalue | padj |
| 1 | NM_173843 | IL1RN (transcript variant 4) | 45.517 | 1.548 | 0.171 | 9.04 | 1.52E−19 | 5.24E−15 |
| 2 | NM_173841 | IL1RN (transcript variant 2) | 40.690 | 1.457 | 0.171 | 8.50 | 1.83E−17 | 3.16E−13 |
| 3 | NM_173842 | IL1RN (transcript variant 1) | 39.568 | 1.448 | 0.171 | 8.45 | 2.88E−17 | 3.30E−13 |
| 4 | NM_000577 | IL1RN (transcript variant 3) | 41.821 | 1.437 | 0.171 | 8.39 | 4.88E−17 | 4.20E−13 |
| 5 | NM_001429 | p300 | 928.435 | 0.955 | 0.171 | 5.57 | 2.50E−08 | 0.000171838 |
| 6 | NM_002253 | KDR | 17.477 | 0.842 | 0.163 | 5.17 | 2.36E−07 | 0.00135472 |
| 7 | NM_030797 | FAM49A | 21.286 | 0.736 | 0.166 | 4.44 | 8.91E−06 | 0.043823927 |
| 8 | NM_012074 | DPF3 | 17.111 | 0.609 | 0.164 | 3.72 | 0.000202676 | 0.871938986 |
| 9 | NM_031476 | CRISPLD2 | 25.148 | 0.569 | 0.167 | 3.41 | 0.000653132 | 0.999954424 |
| 10 | NM_007365 | PADI2 | 99.012 | 0.554 | 0.162 | 3.41 | 0.000641145 | 0.999954424 |

dCas9^{p300 Core (D1399Y)} + 4 IL1RN gRNAs compared to dCas9 + 4 IL1RN gRNAs

| | | | | log2 Fold | | | | |
|---|---|---|---|---|---|---|---|---|
| | Refseq ID | Gene | Base Mean | Change | lfcSE | stat | pvalue | padj |
| 1 | NM_001429 | p300 | 935.659 | 1.234 | 0.198 | 6.24 | 4.36E−10 | 1.49E−05 |
| 2 | NM_001270493 | SREK1 (transcript variant 4) | 30.118 | 0.651 | 0.203 | 3.20 | 0.001388089 | 0.999938051 |
| 3 | NM_001079802 | FKTN (transcript variant 1) | 148.558 | 0.546 | 0.203 | 2.69 | 0.007212168 | 0.999938051 |
| 4 | NM_000922 | PDE3B | 140.122 | 0.535 | 0.201 | 2.66 | 0.007805491 | 0.999938051 |
| 5 | NM_206937 | LIG4 (transcript variant 2) | 30.589 | 0.521 | 0.203 | 2.56 | 0.010513626 | 0.999938051 |
| 6 | NM_001136116 | ZNF879 | 18.421 | 0.520 | 0.201 | 2.59 | 0.009600802 | 0.999938051 |
| 7 | NM_018374 | TMEM106B (transcript | 280.758 | 0.516 | 0.196 | 2.64 | 0.008329592 | 0.999938051 |
| | | variant 1) | | | | | | |
| 8 | NM_019863 | F8 (transcript variant 2) | 8.048 | 0.515 | 0.178 | 2.89 | 0.003827553 | 0.999938051 |
| 9 | NM_001193349 | MEF2C (transcript variant 5) | 18.934 | 0.510 | 0.202 | 2.53 | 0.011492452 | 0.999938051 |
| 10 | NM_183245 | INVS (transcript variant 2) | 38.545 | 0.497 | 0.203 | 2.45 | 0.014125973 | 0.999938051 |

In contrast, dCas9^{p300 Core (D1399Y)} did not significantly induce any IL1RN expression (FIG. 3C; P-value >0.5 for all 4 IL1RN Visoforms). Comparative analysis to dCas9 revealed limited dCas9^{p300 Core} off-target gene induction, with only two transcripts induced significantly above background at a false discovery rate (FDR)<5%: KDR (FDR=1.4×10⁻³); and FAM49A (FDR=0.04) (FIG. 3B, Table 6). Increased expression of p300 mRNA was observed in cells transfected with dCas9^{p300 Core} and dCas9^{p300 Core (D1399Y)}. This finding is most likely explained by RNA-seq reads mapping to mRNA from the transiently transfected p300 core fusion domains. Thus the dCas9^{p300 Core} fusion displayed high genome-wide targeted transcriptional specificity and robust gene induction of all four targeted IL1RN isoforms.

### Example 5

### dCas9^{p300 Core} Acetylates H3K27 at Enhancers and Promoters

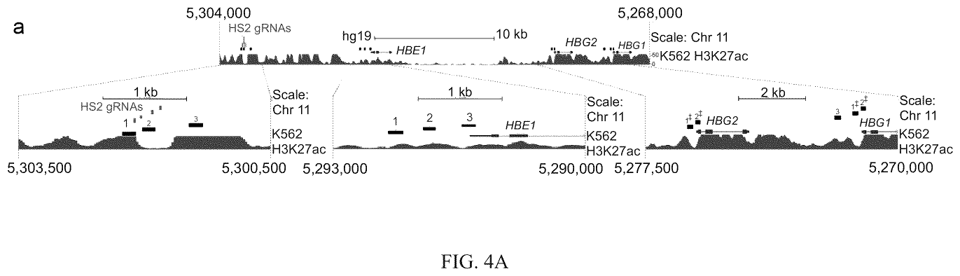

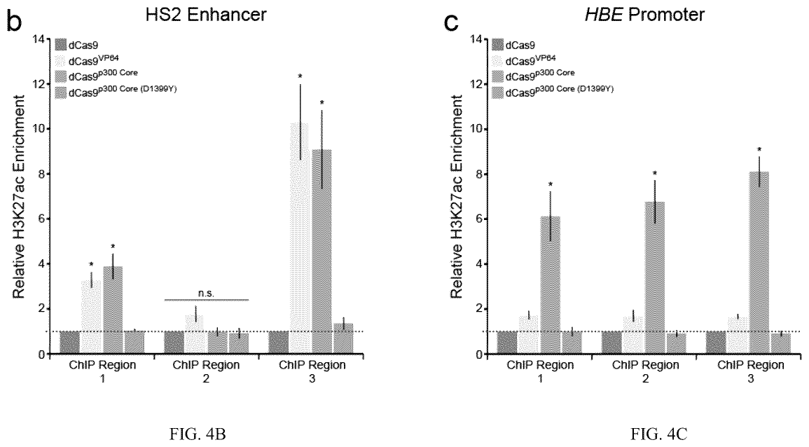

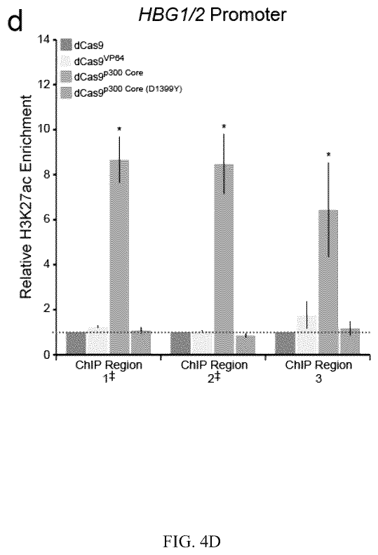

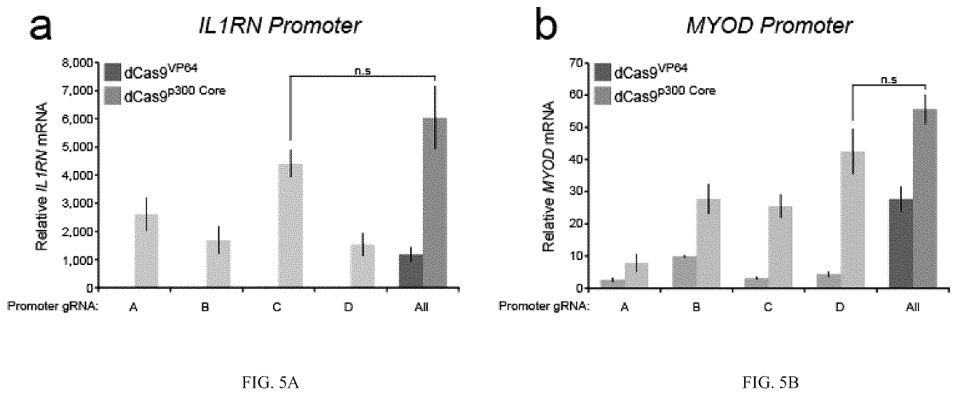

Activity of regulatory elements correlates with covalent histone modifications such as acetylation and methylation. Of those histone modifications, acetylation of lysine 27 on histone H3 (H3K27ac) is one of the most widely documented indicators of enhancer activity. Acetylation of H3K27 is catalyzed by p300 and is also correlated with endogenous p300 binding profiles. Therefore H3K27ac enrichment was used as a measurement of relative dCas9^{p300 Core}-mediated acetylation at the genomic target site. To quantify targeted H3K27 acetylation by dCas9^{p300 Core} chromatin immuno-precipitation was performed with an anti-H3K27ac antibody followed by quantitative PCR (ChIP-qPCR) in HEK293T cells co-transfected with four HS2 enhancer-targeted gRNAs and either dCas9, dCas9^{VP64} dCas9^{p300 Core} or dCas9^{p300 Core (D1399Y)} (FIGS. 4A-4D). Three amplicons were analyzed at or around the target site in the HS2 enhancer or within the promoter regions of the HBE and HBG genes (FIG. 4A). Notably, H3K27ac is enriched in each of these regions in the human K562 erythroid cell line that has a high level of globin gene expression (FIG. 4A). Significant H3K27ac enrichment was observed at the HS2 enhancer target locus compared to treatment with dCas9 in both the dCas9^{VP64} (P-value 0.0056 for ChIP Region land P-value 0.0029 for ChIP Region 3) and dCas9^{p300 Core} (P-value 0.0013 for ChIP Region land P-value 0.0069 for ChIP Region 3) co-transfected samples (FIG. 4B).

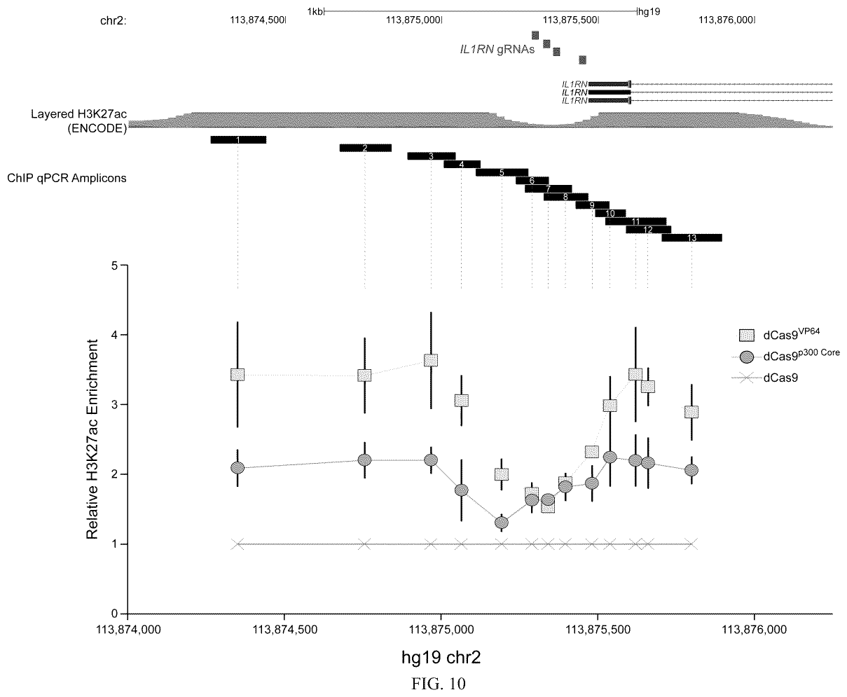

A similar trend of H3K27ac enrichment was also observed when targeting the IL1RN promoter with dCas9^{VP64} or dCas9^{p300 Core} (FIG. 10). FIG. 10 shows the IL1RN locus on GRCh37/hg19 along with IL1RN gRNA target sites. In addition, layered ENCODE H3K27ac enrichment from seven cell lines (GM12878, H1-hESC, HSMM, HUVEC, K562, NHEK, and NHLF) is indicated with the vertical range setting set to 50. Tiled IL1RN ChIP qPCR amplicons (1-13) are also shown in corresponding locations on GRCh37/hg19. H3K27ac enrichment for dCas9^{VP64} and dCas9^{p300 Core} co-transfected with four IL1RN-targeted gRNAs and normalized to dCas9 co-transfected with four IL1RN gRNAs is indicated for each ChIP qPCR locus assayed. 5ng of ChIP-prepared DNA was used for each reaction (n=3 independent experiments, error bars: s.e.m.).

In contrast to these increases in H3K27ac at the target sites by both dCas9^{VP64} or dCas9^{p300 Core} robust enrichment in H3K27ac at the HS2-regulated HBE and HBG promoters was observed only with dCas9^{p300 Core} treatment (FIGS. 4C-4D). Together these results demonstrate that dCas9^{p300 Core} uniquely catalyzes H3K27ac enrichment at gRNA-targeted loci and at enhancer-targeted distal promoters. Therefore the acetylation established by dCas9^{p300 Core} at HS2 may catalyze enhancer activity in a manner distinct from direct recruitment of preinitiation complex components by dCas9^{VP64}, as indicated by the distal activation of the HBE, HBG, and HBD genes from the HS2 enhancer by dCas9^{p300 Core} but not by dCas9^{VP64} (FIG. 2C, FIGS. 9A-9E).

### Example 6

### dCas9^{p300 Core} Activates Genes with a Single gRNA

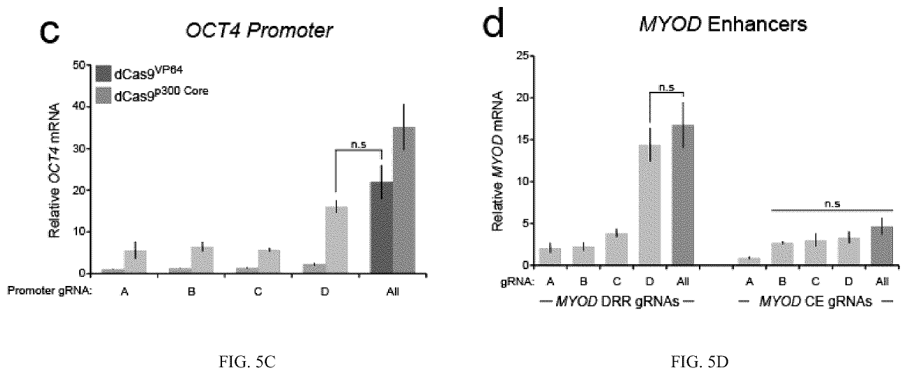

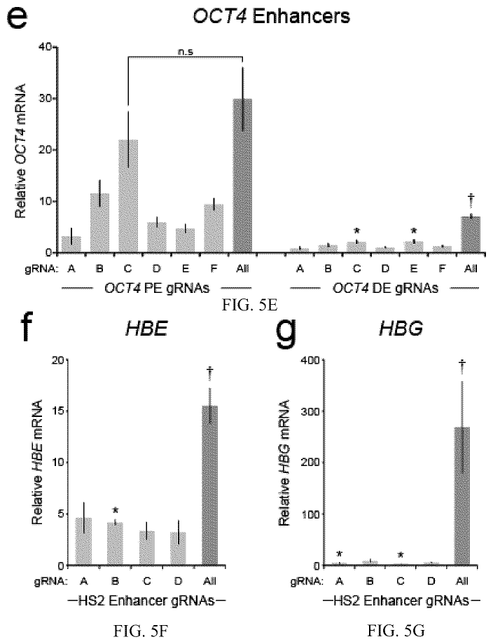

Robust transactivation using dCas9-effector fusion proteins currently relies upon the application of multiple gRNAs, multiple effector domains, or both. Transcriptional activation could be simplified with the use of single gRNAs in tandem with a single dCas9-effector fusion. This would also facilitate multiplexing distinct target genes and the incorporation of additional functionalities into the system. The transactivation potential of dCas9^{p300 Core} with single gRNAs was compared to that of dCas9^{p300 Core} with four pooled gRNAs targeting the IL1RN, MYOD and OCT4 promoters (FIGS. 5A-5B). Substantial activation was observed upon co-transfection of the dCas9^{p300 Core} and a single gRNA at each promoter tested. For the IL1RN and MYOD promoters, there was no significant difference between the pooled gRNAs and the best individual gRNA (FIGS. 5A-5B; IL1RN gRNA “C”, P-value 0.78; MYOD gRNA “D”, P-value 0.26). Although activation of the OCT4 promoter produced additive effects when four gRNAs were pooled with dCas9^{p300 Core} the most potent single gRNA (gRNA “D”) induced a statistically comparable amount of gene expression to that observed upon co-transfection of dCas9^{VP64} with an equimolar pool of all four promoter gRNAs (P-value 0.73; FIG. 5C). Compared to dCas9^{p300 Core} levels of gene activation with dCas9^{VP64} and single gRNAs were substantially lower. Also, in contrast to dCas9^{p300 Core}, dCas9^{VP64} demonstrated synergistic effects with combinations of gRNAs in every case (FIGS. 5A-5C).

Based on the transactivation ability of dCas9^{p300 Core} at enhancer regions and with single gRNAs at promoter regions, it was hypothesized that dCas9^{p300 Core} might also be able to transactivate enhancers via a single targeted gRNA. The MYOD (DRR and CE), OCT4 (PE and DE), and HS2 enhancer regions were tested with equimolar concentrations of pools or single gRNAs (FIGS. 5D-5G). For both MYOD enhancer regions, co-transfection of dCas9^{p300 Core} and a single enhancer-targeting gRNA was sufficient to activate gene expression to levels similar to cells co-transfected with dCas9^{p300 Core} and the four pooled enhancer gRNAs (FIG. 5D). Similarly, OCT4 gene expression was activated from the PE via dCas9^{p300 Core} localization with a single gRNA to similar levels as dCas9^{p300 Core} localized with a pool of six PE-targeted gRNAs (FIG. 5E). dCas9^{p300 Core}-mediated induction of OCT4 from the DE (FIG. 5E) and HBE and HBG genes from the HS2 enhancer (FIGS. 5F-5G) showed increased expression with the pooled gRNAs relative to single gRNAs. Nevertheless, there was activation of target gene expression above control for several single gRNAs at these enhancers (FIGS. 5E-5G).

### Example 7

### The p300 HAT Domain is Portable to Other DNA-Binding Proteins

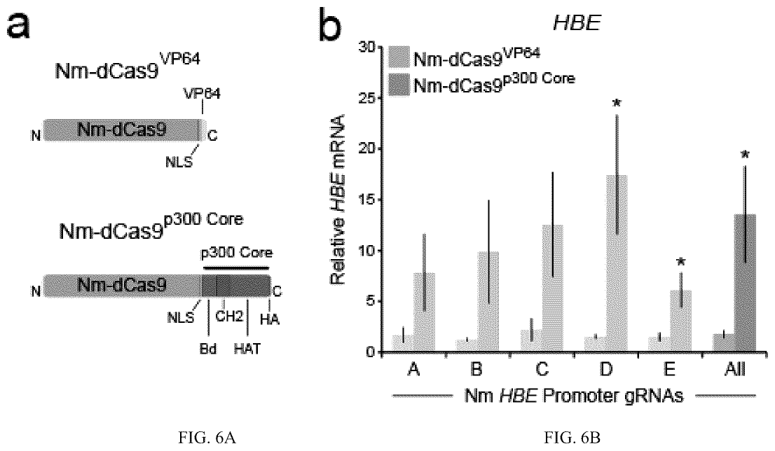

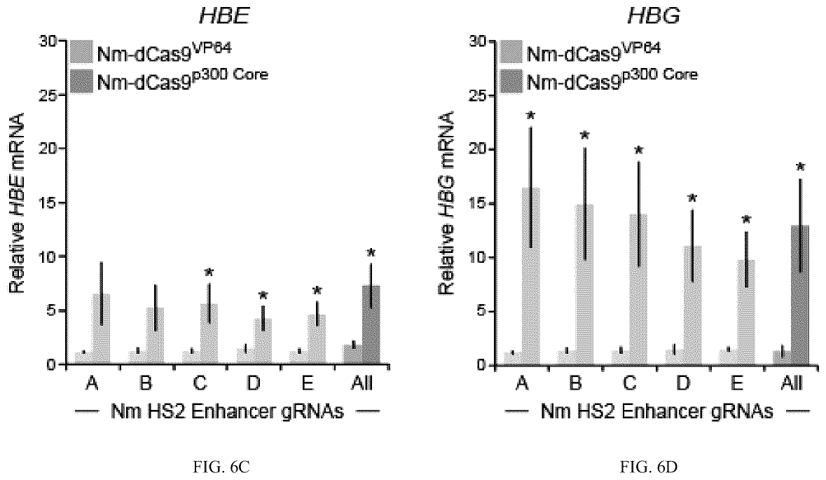

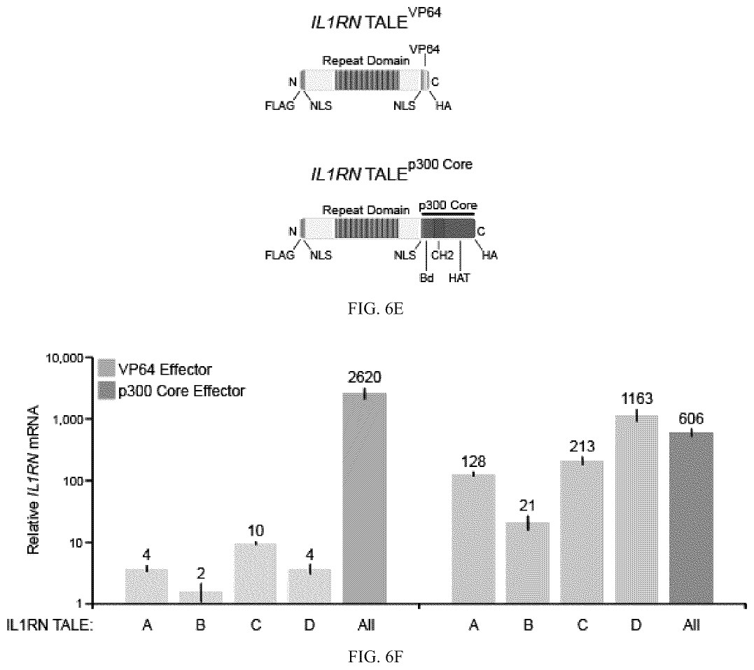

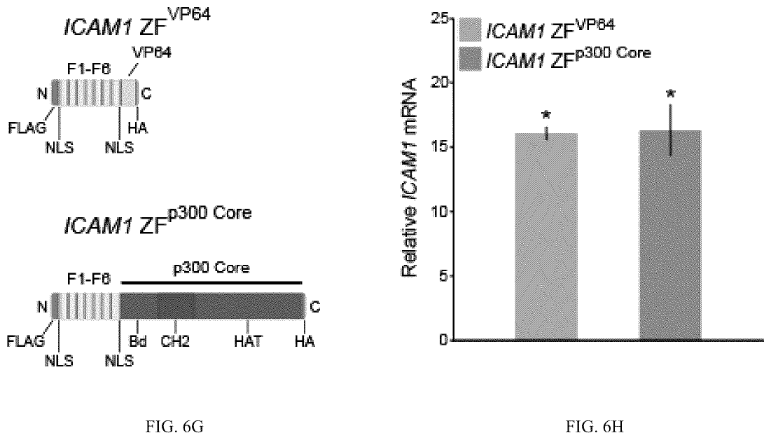

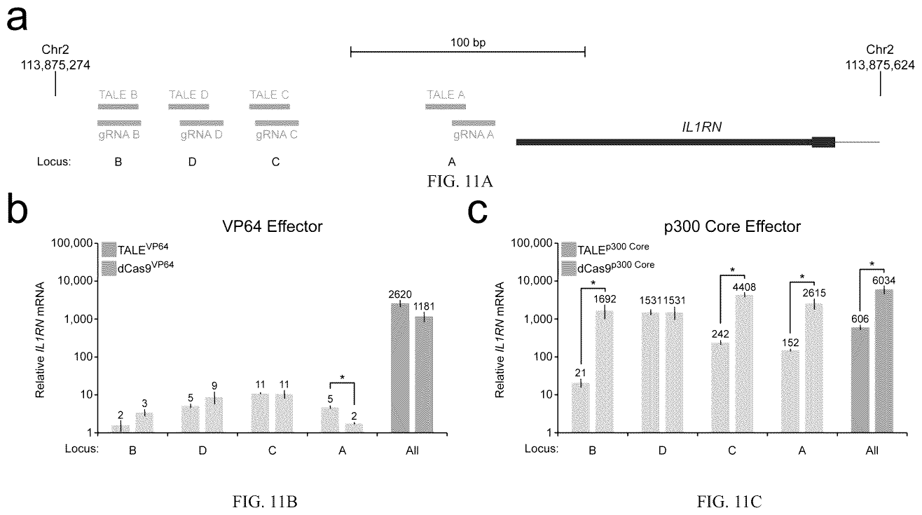

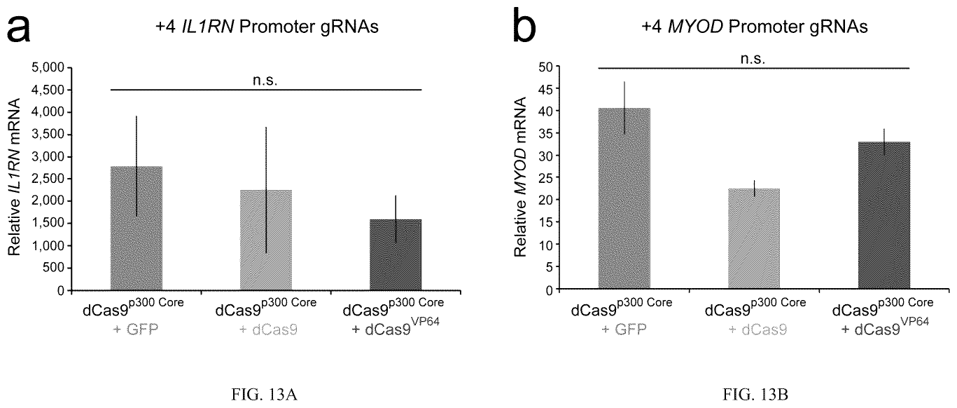

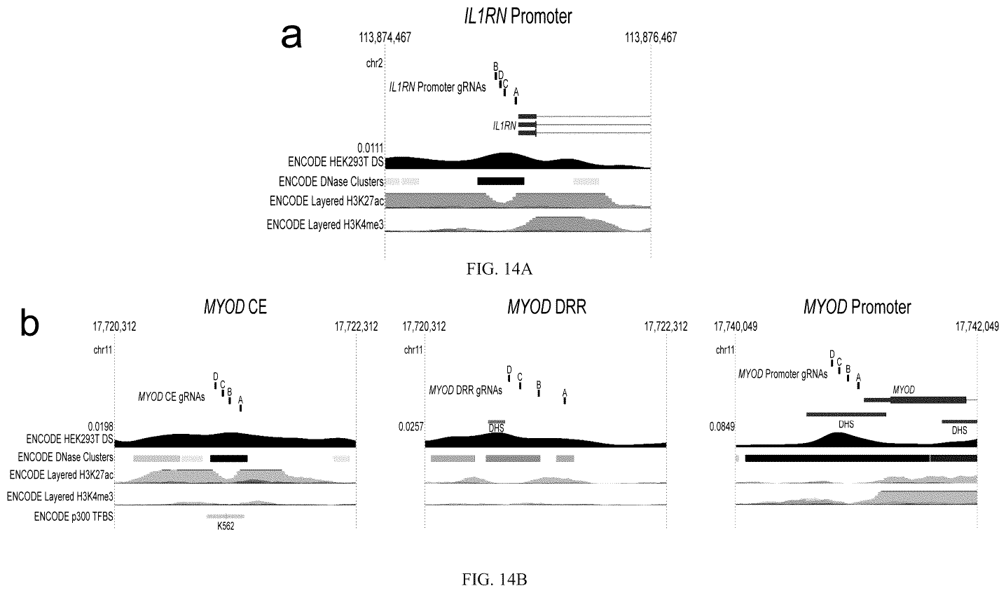

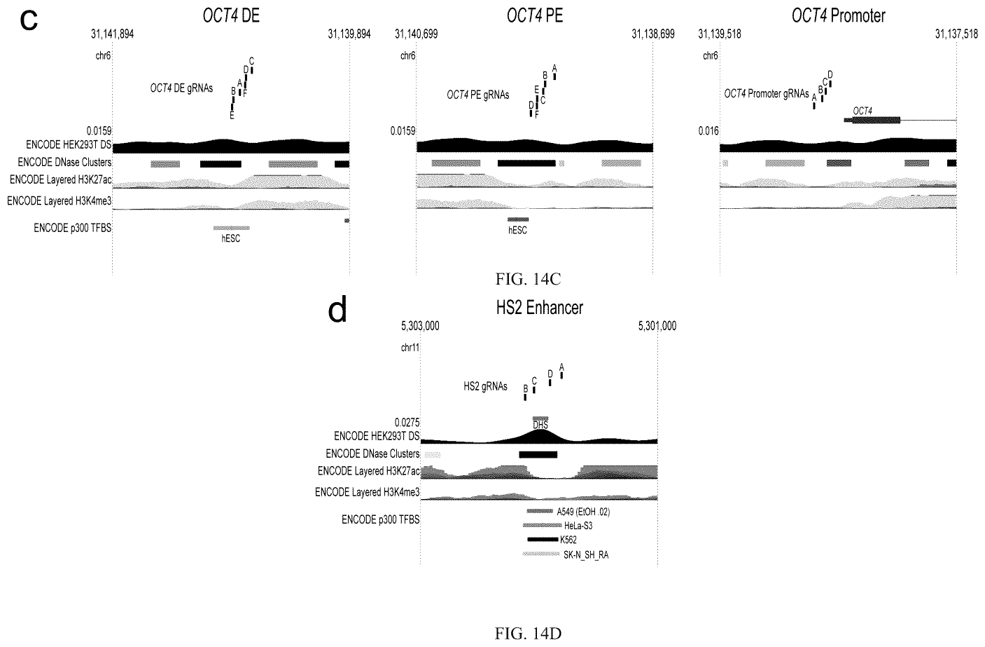

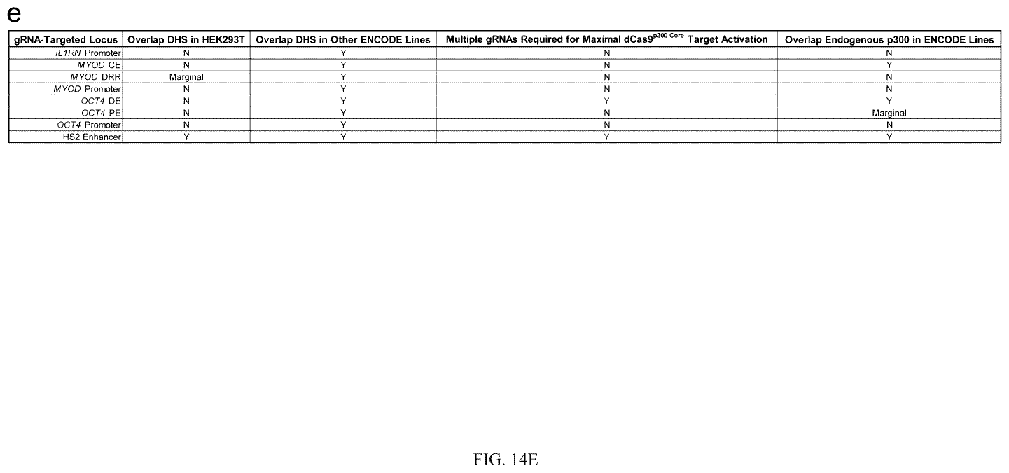

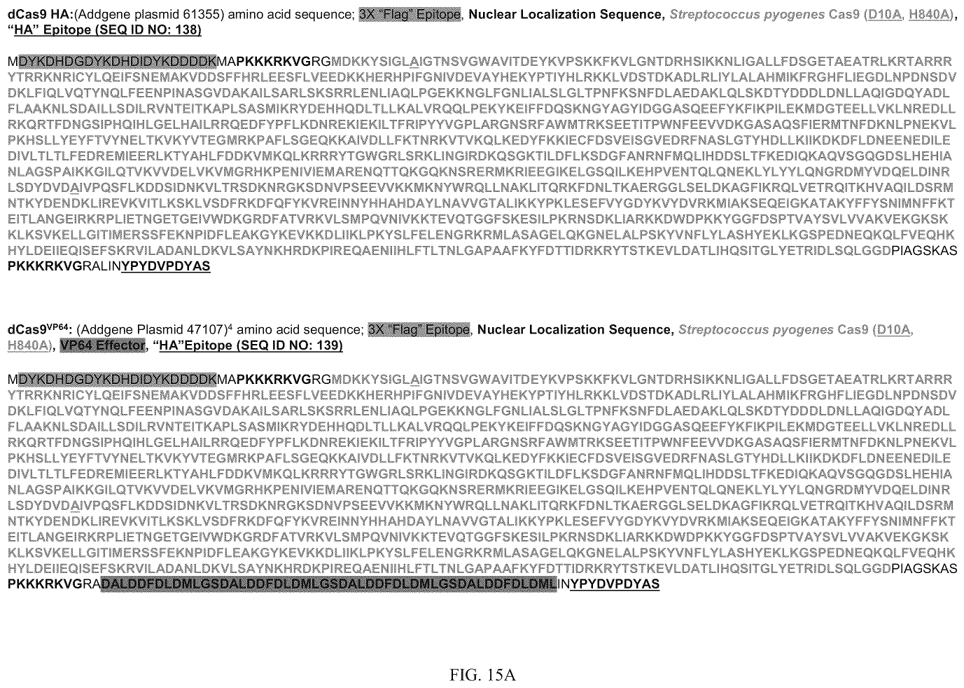

The dCas9/gRNA system from Streptococcus pyogenes has been widely adopted due to its robust, versatile, and easily programmable properties. However, several other programmable DNA-binding proteins are also under development for various applications and may be preferable for particular applications, including orthogonal dCas9 systems from other species, TALEs, and zinc finger proteins. To determine if the p300 Core HAT domain was portable to these other systems, fusions were created to dCas9 from Neisseria meningitidis (Nm-dCas9), four different TALEs targeting the IL1RN promoter, and a zinc finger protein targeting ICAM1 (FIGS. 6A-6H). Co-transfection of Nm-dCas9^{p300 Core} and five Nm-gRNAs targeted to the HBE or the HBG promoters led to significant gene induction compared to mock-transfected controls (P-value 0.038 and 0.0141 for HBE and HBG respectively) (FIG. 6B). When co-transfected with five Nm-gRNAs targeted to the HS2 enhancer, Nm-dCas9^{p300 Core} also significantly activated the distal HBE and HBG, globin genes compared to mock-transfected controls (p=0.0192 and p=0.0393, respectively) (FIGS. 6C-6D). Similar to dCas9^{p300 Core}, Nm-dCas9^{p300 Core} activated gene expression from promoters and the HS2 enhancer via a single gRNA. Nm-dCas9^{VP64} displayed negligible capacity to transactivate HBE or HBG regardless of localization to promoter regions or to the HS2 enhancer either with single or multiple gRNAs (FIGS. 6B-6D). Transfection of the expression plasmids for a combination of four TALE^{p300 Core} fusion proteins targeted to the IL1RN promoter (IL1RN TALE^{p300 Core}) also activated downstream gene expression, although to a lesser extent than four corresponding TALE^{VP64} fusions (IL1RN TALE^{VP64}) (FIGS. 6E-6F). However, single p300 Core effectors were much more potent than single VP64 domains when fused to IL1RN TALEs. Interestingly, dCas9^{p300 Core} directed to any single binding site generated comparable IL1RN expression relative to single or pooled IL1RN TALE effectors and direct comparisons suggest that dCas9 may be a more robust activator than TALEs when fused to the larger p300 Core fusion domain (FIGS. 11A-11C). The p300 Core effector domain did not display synergy with either additional gRNAs or TALEs (see FIGS. 5A-5G, 6A-6H, 9A-9E, and 11A-11C) or in combination with VP64 (see FIGS. 13A-13B). The underlying chromatin context of the dCas9p300 Core target loci is shown in FIGS. 14A-14E.

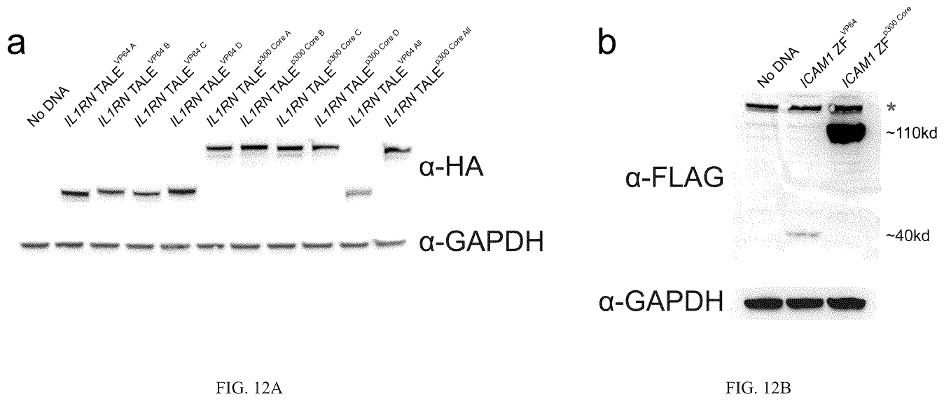

The ZF^{p300 Core} fusion targeted to the ICAM1 promoter (ICAM1 ZF^{p300 Core}) also activated its target gene relative to control and at a similar level as ZF^{VP64} (ICAM1 ZF^{VP64}) (FIGS. 6G-6H). The versatility of the p300 Core fusion with multiple targeting domains is evidence that this is a robust approach for targeted acetylation and gene regulation. The various p300 core fusion proteins were expressed well, as determined by western blot (FIGS. 12A-12B), but differences in p300 Core activity between different fusion proteins could be attributable to binding affinity or protein folding.

### Example 8

### Myocardin

36 gRNAs were designed to span −2000 bp to +250 bp (coordinates relative to TSS) region of the MYOCD gene (Table 7).

TABLE 7
Myocd gRNAs Information

| | | | SEQ | | SEQ | | | Coordinates | |
|---|---|---|---|---|---|---|---|---|---|
| Target | gRNA | | ID | | ID | | | Relative to | |
| Name | Name | Protospacer (N20) | NO | PAM | NO | +/− | Length | TSS | |
| Myocd | Cr1 | cctggtcttcaatgagaaga | 152 | NGG | 188 | − | 20 | −1991 | −1971 |
| Myocd | Cr2 | gattaggacatgaacatggg | 153 | NGG | 189 | − | 20 | −1897 | −1877 |
| Myocd | Cr3 | cctcttctacattaacctta | 154 | NGG | 190 | − | 20 | −1771 | −1751 |
| Myocd | Cr4 | tttttgaagccagcaatcgt | 155 | NGG | 191 | − | 20 | −1693 | −1673 |
| Myocd | Cr5 | cgttagtttctggaggctct | 156 | NGG | 192 | − | 20 | −1597 | −1577 |
| Myocd | Cr6 | acaaattaccacgaatgtag | 157 | NGG | 193 | − | 20 | −1480 | −1460 |
| Myocd | Cr7 | tggcctgggcgcctgtctat | 158 | NGG | 194 | − | 20 | −1395 | −1375 |
| Myocd | Cr8 | attttgtaaataaggtcttc | 159 | NGG | 195 | − | 20 | −1297 | −1277 |
| Myocd | Cr9 | agcaacaggggatggggcag | 160 | NGG | 196 | + | 20 | −1221 | −1201 |
| Myocd | Cr10 | aggactcgtagtatgcaggc | 161 | NGG | 197 | + | 20 | −1120 | −1100 |
| Myocd | Cr11 | ctgagccaccaactatttaa | 162 | NGG | 198 | + | 20 | −1005 | −985 |
| Myocd | Cr12 | ctgagccaccaactatttaa | 163 | NGG | 199 | + | 20 | −945 | −925 |
| Myocd | Cr13 | actctgggtcggttacggaa | 164 | NGG | 200 | + | 20 | −907 | −887 |
| Myocd | Cr14 | gggctgggcttagcttggga | 165 | NGG | 201 | − | 20 | −837 | −817 |
| Myocd | Cr15 | atagggaggggctctggagc | 166 | NGG | 202 | − | 20 | −798 | −778 |
| Myocd | Cr16 | atgggaaaagatacctgagt | 167 | NGG | 203 | − | 20 | −751 | −731 |
| Myocd | Cr17 | tgggagcgttgtgtcgcagc | 168 | NGG | 204 | + | 20 | −713 | −693 |
| Myocd | Cr18 | tggaaaggctttcattttct | 169 | NGG | 205 | − | 20 | −642 | −622 |
| Myocd | Cr19 | gtatctcgcagctccaatac | 170 | NGG | 206 | − | 20 | −594 | −574 |
| Myocd | Cr20 | acgcattcccctcggtttga | 171 | NGG | 207 | − | 20 | −544 | −524 |
| Myocd | Cr21 | tcggaagcttttcttctcag | 172 | NGG | 208 | + | 20 | −511 | −491 |
| Myocd | Cr22 | cgaaagggcgtgcgcgcccg | 173 | NGG | 209 | − | 20 | −449 | −429 |
| Myocd | Cr23 | ccggcgaaagggaagcggcc | 174 | NGG | 210 | − | 20 | −396 | −376 |
| Myocd | Cr24 | ggctgcgcacgcccatcccc | 175 | NGG | 211 | + | 20 | −352 | −332 |
| Myocd | Cr25 | ggggcttgcaggtggttcgc | 176 | NGG | 212 | − | 20 | −297 | −277 |
| Myocd | Cr26 | cgagctaaagagcggatgcc | 177 | NGG | 213 | − | 20 | −246 | −226 |
| Myocd | Cr27 | agagggcgggagcagggcca | 178 | NGG | 214 | − | 20 | −200 | −180 |
| Myocd | Cr28 | aaccggctcttaactctttg | 179 | NGG | 215 | − | 20 | −153 | −133 |
| Myocd | Cr29 | caggagcggcgagcggggtc | 180 | NGG | 216 | − | 20 | −101 | −81 |
| Myocd | Cr30 | gggtatcagatggcaaagtt | 181 | NGG | 217 | + | 20 | −54 | −34 |
| Myocd | Cr31 | tcataggctgccggcgattg | 182 | NGG | 218 | − | 20 | 0 | 20 |
| Myocd | Cr32 | gaggttggccaggagcagcg | 183 | NGG | 219 | − | 20 | 47 | 67 |
| Myocd | Cr33 | aattagccccgcacggcgag | 184 | NGG | 220 | + | 20 | 100 | 120 |
| Myocd | Cr34 | tcccctgggtaggagtacag | 185 | NGG | 221 | − | 20 | 157 | 177 |
| Myocd | Cr35 | ggttgttagctgcggtcagc | 186 | NGG | 222 | + | 20 | 203 | 223 |
| Myocd | Cr36 | ggtggagaacagggggcgcc | 187 | NGG | 223 | + | 20 | 246 | 266 |

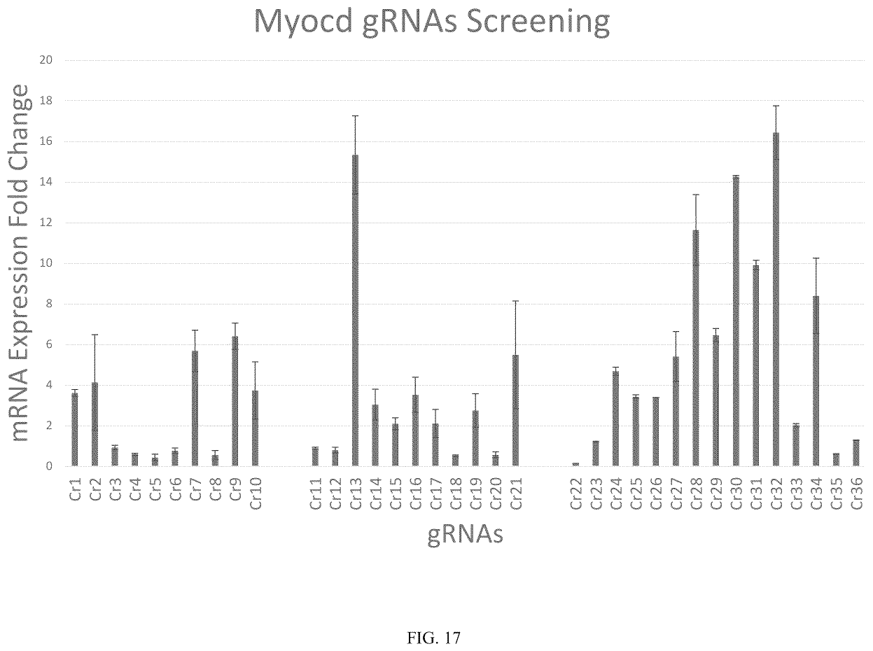

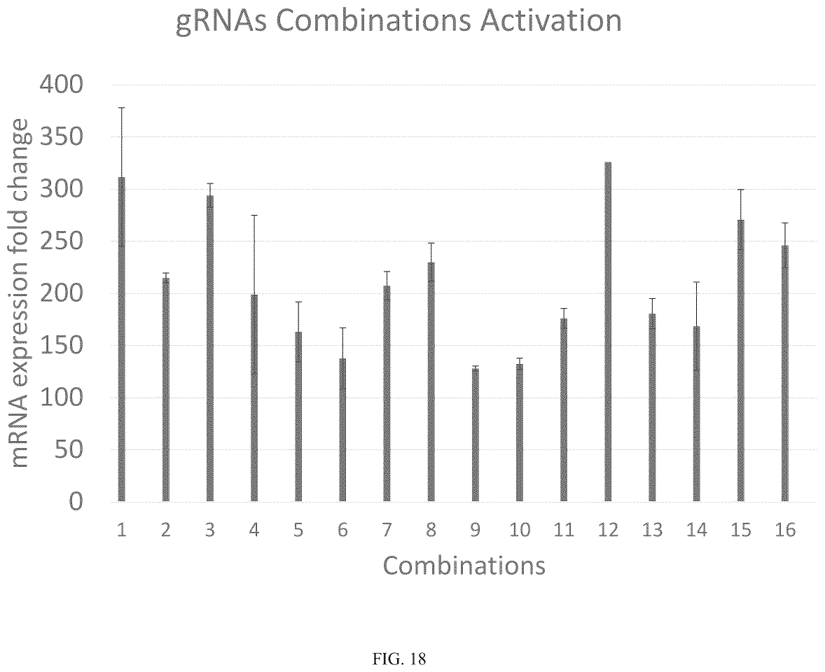

The gRNAs were cloned into a spCas9 gRNA expression vector containing hU6 promoter and BbsI restriction site. The gRNAs were transiently co-transfected with dCas9^{p300 Core} into BTEK293T cells. The resulting mRNA production for myocardin was assayed in samples harvested three days post-transfection (FIG. 17). Combinations of Cr32, Cr13, Cr30, Cr28, Cr31, and Cr34 were analyzed with dCas9^{p300 Core} (Table 8; FIG. 18).

TABLE 8

| Condition | Cr32 | Cr13 | Cr30 | Cr28 | Cr31 | Cr34 |
|---|---|---|---|---|---|---|
| 1 | X | X | X | X | X | X |
| 2 | X | X | X | X | | |
| 3 | X | X | X | | X | |
| 4 | X | X | X | | | X |
| 5 | X | X | | X | X | |
| 6 | X | X | | | X | X |
| 7 | X | X | | X | | X |
| 8 | X | | X | X | X | |
| 9 | | X | X | X | | X |
| 10 | | X | | X | X | X |
| 11 | | X | X | X | X | X |
| 12 | X | | X | X | X | X |
| 13 | X | X | | X | X | X |
| 14 | X | X | X | | X | X |
| 15 | X | X | X | X | | X |
| 16 | X | X | X | X | X | |

### Example 9

### Pax7

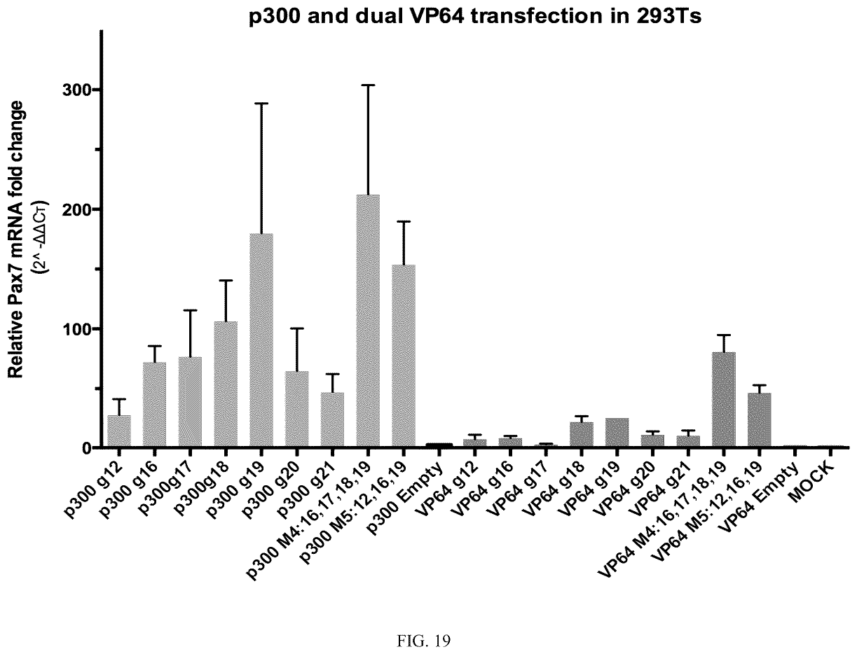

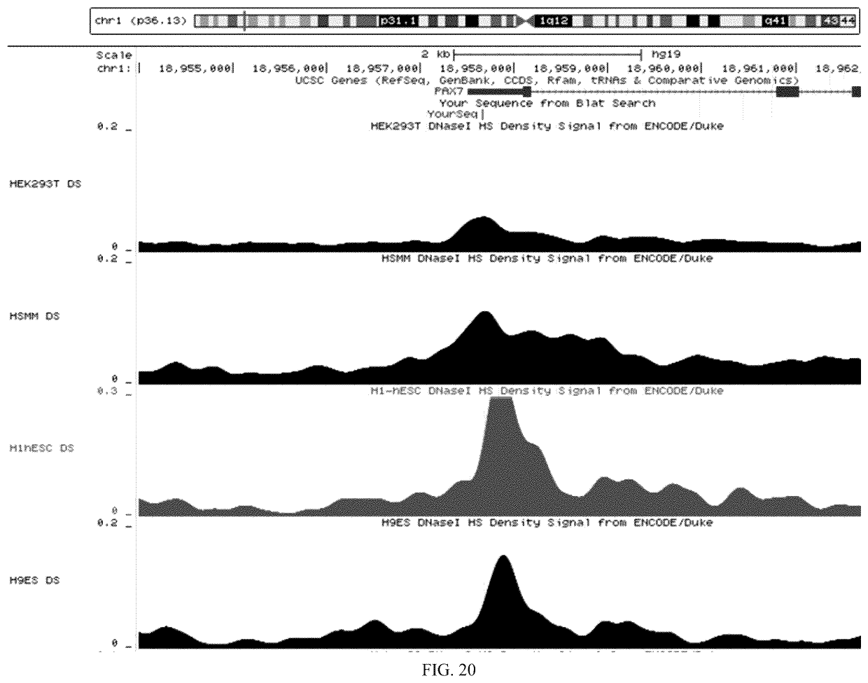

gRNAs were designed to span the region surrounding the PAX7 gene (Table 9). The gRNAs were cloned into a spCas9 gRNA expression vector containing hU6 promoter and BbsI restriction site. The gRNAs were transiently co-transfected with dCas9^{p300 Core} or dCas9^{VP64} into HEK293T cells. The resulting mRNA production for Pax7 was assayed in samples harvested three days post-transfection (FIG. 19). The gRNA19 (“g19”) was used in further experiments and shown to localize to a DNase hypersensitive site (DHS) (FIG. 20).

TABLE 9
Pax7 gRNAs

| TSS Target | | Target | | SEQ ID |
|---|---|---|---|---|
| position | Strand | name | Oligo in sense strand | NO |
| 138 | AS | JK12 | GGGGGCGCGAGTGATCAGCT | 224 |
| 27 | S | JK16 | CCCGGGTCTCCTAGGGGACG | 225 |
| +95 | S | JK17 | TGGTCCGGAGAAAGAAGGCG | 226 |
| +187 | S | JK18 | GTCTCCGGGCTCGGAAACTT | 227 |
| +223 | S | JK19 | AGCGCCAGAGCGCGAGAGCG | 228 |
| +273 | S | JK20 | CGATTCCGGCCGCGTTCCCC | 229 |
| +335 | AS | JK21 | GTTGTGCGGGCTGATGCGCC | 230 |

### Example 10

### FGF1

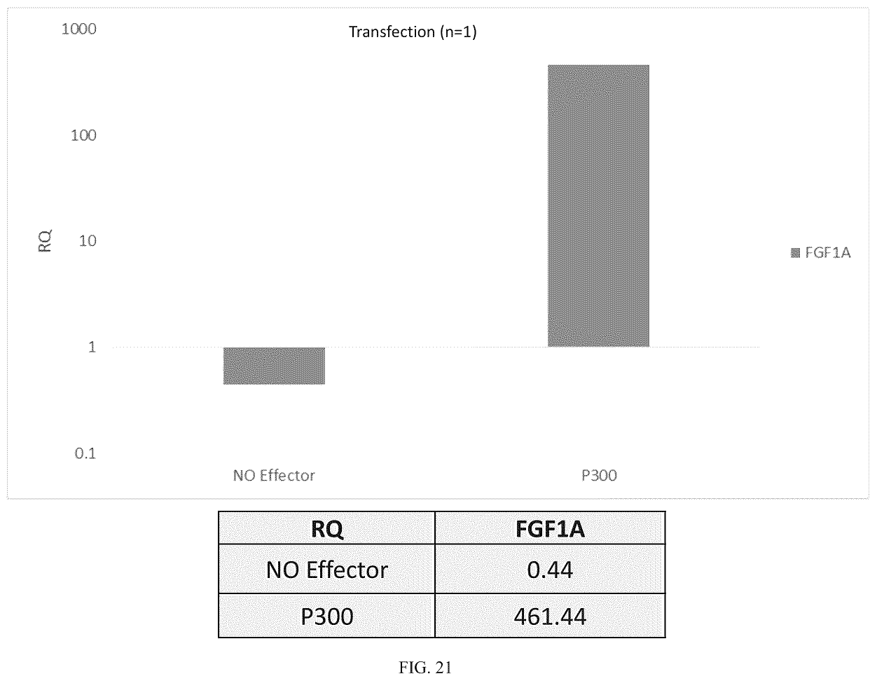

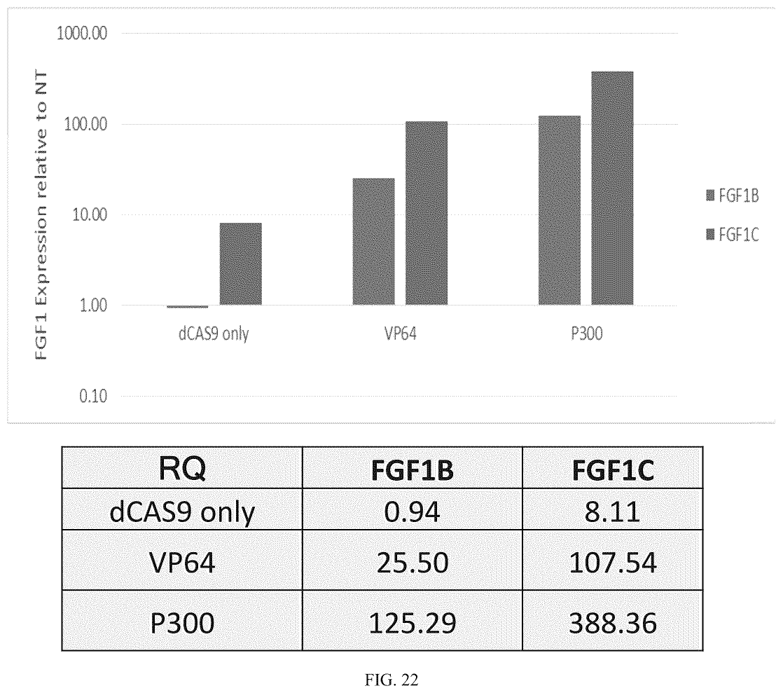

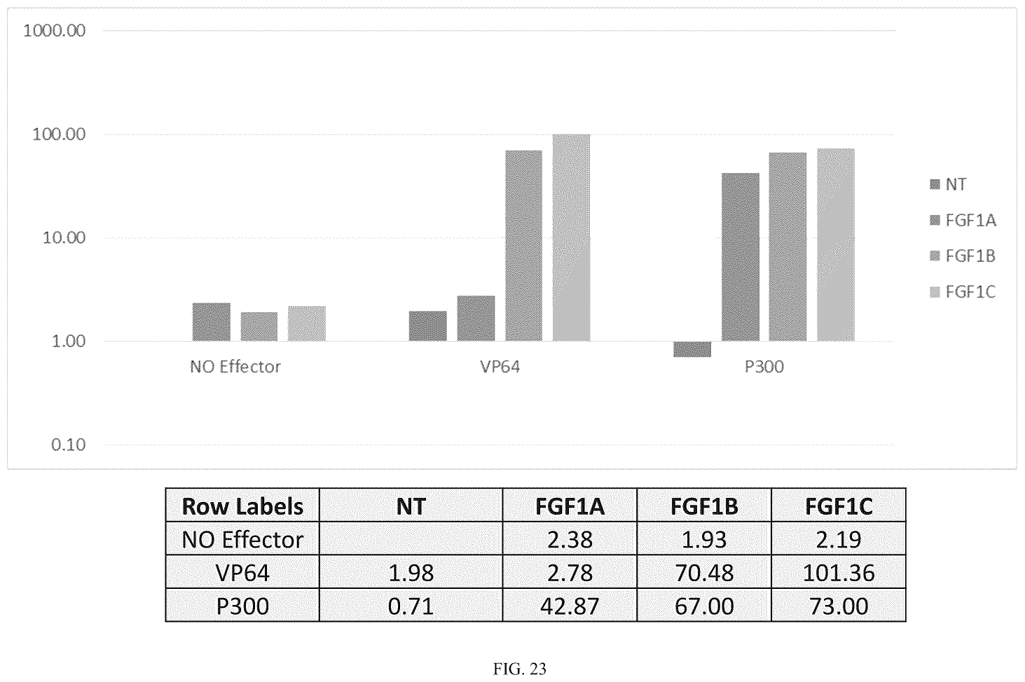

gRNAs were designed for the FGF1A, FGF1B, and FGF1C genes (Tables 10 and 11). The 25 nM of gRNAs were transiently co-transfected with dCas9^{p300 Core} or dCas9^{VP64} into HEK293T cells. The resulting mRNA production for FGF1 expression was determined (FIGS. 21-23). In FIG. 23, the number of stable cell-lines transfected with the lentivirus vector was 2, except for FGF1A where n=1.

TABLE 10

| gRNA | Gene | Type | Name |
|---|---|---|---|
| 1 | FGF1A | F_7sk | 1FGF1AF_7sk |
| 2 | FGF1A | F_h1 | 2FGF1AF_h1 |
| 3 | FGF1A | F_hU6 | 3FGF1AF_hU6 |
| 4 | FGF1A | F_mU6 | 4FGF1AF_mU6 |
| 1 | FGF1A | R_7sk | 1FGF1AR_7sk |
| 2 | FGF1A | R_h1 | 2FGF1AR_h1 |
| 3 | FGF1A | R_hU6 | 3FGF1AR_hU6 |
| 4 | FGF1A | R_mU6 | 4FGF1AR_mU6 |
| 1 | FGF1B | F_7sk | 1FGF1BF_7sk |
| 2 | FGF1B | F_h1 | 2FGF1BF_h1 |
| 3 | FGF1B | F_hU6 | 3FGF1BF_hU6 |
| 4 | FGF1B | F_mU6 | 4FGF1BF_mU6 |
| 1 | FGF1B | R_7sk | 1FGF1BR_7sk |
| 2 | FGF1B | R_h1 | 2FGF1BR_h1 |
| 3 | FGF1B | R_hU6 | 3FGF1BR_hU6 |
| 4 | FGF1B | R_mU6 | 4FGF1BR_mU6 |
| 1 | FGF1C | F_7sk | 1FGF1CF_7sk |
| 2 | FGF1C | F_h1 | 2FGF1CF_h1 |
| 3 | FGF1C | F_hU6 | 3FGF1CF_hU6 |
| 4 | FGF1C | F_mU6 | 4FGF1CF_mU6 |
| 1 | FGF1C | R_7sk | 1FGF1CR_7sk |
| 2 | FGF1C | R_h1 | 2FGF1CR_h1 |
| 3 | FGF1C | R_hU6 | 3FGF1CR_hU6 |
| 4 | FGF1C | R_mU6 | 4FGF1CR_mU6 |

TABLE 11
FGF1 gRNAs Information

| | | SEQ | | | SEQ | |
|---|---|---|---|---|---|---|
| | | ID | 1st | | ID | 2nd |
| gRNA | Final Sequence | NO | addition | Sequence | NO | addition |
| 1 | CCTCGTGTGTTCCTGGGC | 231 | CCTCG | TGTGTTCCTGGGCCTG | 255 | |
| | CTGCTGC | | | CTGC | | |
| 2 | TCCCATAAACAGGATTC | 232 | TCCCA | TAAACAGGATTCTGCT | 256 | |
| | TGCTCAGA | | | CAGA | | |
| 3 | CACCGGCCAGATGACAG | 233 | CACCG | GCCAGATGACAGAAC | 257 | |
| | AACAGAAA | | | AGAAA | | |
| 4 | TTGTTTGAAAATGCCATT | 234 | TTGTTTG | AAAATGCCATTTGTA | 258 | |
| | TGTAGGGCT | | | GGGCT | | |
| 1 | AAACGCAGCAGGCCCAG | 235 | AAAC | GCAGCAGGCCCAGGA | 259 | C |
| | GAACACAC | | | ACACA | | |
| 2 | AAACTCTGAGCAGAATC | 236 | AAAC | TCTGAGCAGAATCCT | 260 | T |
| | CTGTTTAT | | | GTTTA | | |
| 3 | AAACTTTCTGTTCTGTCA | 237 | AAAC | TTTCTGTTCTGTCATC | 261 | C |
| | TCTGGCC | | | TGGC | | |
| 4 | AAACAGCCCTACAAATG | 238 | AAAC | AGCCCTACAAATGGC | 262 | CAA |
| | GCATTTTCAA | | | ATTTT | | |
| 1 | CCTCGtctgcttctgccgaacctca | 239 | CCTCG | tctgcttctgccgaacctca | 263 | |
| 2 | TCCCAcctaaagagcttgtaggccg | 240 | TCCCA | cctaaagagcttgtaggccg | 264 | |
| 3 | CACCGagagctggctacccgtccct | 241 | CACCG | agagctggctacccgtccct | 265 | |
| 4 | TTGTTTGcggtccttgtttatcagta | 242 | TTGTTTG | cggtccttgtttatcagtag | 266 | |
| | g | | | | | |
| 1 | AAACtgaggttcggcagaagcaga | 243 | AAAC | tgaggttcggcagaagcaga | 267 | C |
| | C | | | | | |
| 2 | AAACcggcctacaagctctttaggT | 244 | AAAC | cggcctacaagctctttagg | 268 | T |
| 3 | AAACagggacgggtagccagctct | 245 | AAAC | agggacgggtagccagctct | 269 | C |
| | C | | | | | |
| 4 | AAACctactgataaacaaggaccgC | 246 | AAAC | ctactgataaacaaggaccg | 270 | CAA |
| | AA | | | | | |
| 1 | CCTCGGAGCTGGCTACC | 247 | CCTCG | GAGCTGGCTACCCGT | 271 | |
| | CGTCCCTA | | | CCCTA | | |
| 2 | TCCCACTTTGGCTGGGTT | 248 | TCCCA | CTTTGGCTGGGTTTAA | 272 | |
| | TAAACCA | | | ACCA | | |
| 3 | CACCGGTCAGCTCAGGG | 249 | CACCG | GTCAGCTCAGGGTTTT | 273 | |
| | TTTTGGTA | | | GGTA | | |
| 4 | TTGTTTGGAGTTAGCTCC | 250 | TTGTTTG | GAGTTAGCTCCCCGA | 274 | |
| | CCGACCCAG | | | CCCAG | | |
| 1 | AAACTAGGGACGGGTAG | 251 | AAAC | TAGGGACGGGTAGCC | 275 | C |
| | CCAGCTCC | | | AGCTC | | |
| 2 | AAACTGGTTTAAACCCA | 252 | AAAC | TGGTTTAAACCCAGCC | 276 | T |
| | GCCAAAGT | | | AAAG | | |
| 3 | AAACTACCAAAACCCTG | 253 | AAAC | TACCAAAACCCTGAG | 277 | C |
| | AGCTGACC | | | CTGAC | | |
| 4 | AAACCTGGGTCGGGGAG | 254 | AAAC | CTGGGTCGGGGAGCT | 278 | CAA |
| | CTAACTCCAA | | | AACTC | | |

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, various aspects of the invention are set out in the following numbered clauses:

Clause 1. A fusion protein comprising two heterologous polypeptide domains, wherein the first polypeptide domain comprises a Clustered Regularly Interspaced Short Palindromic Repeats associated (Cas) protein and the second polypeptide domain comprises a peptide having histone acetyltransferase activity.

Clause 2. The fusion protein of clause 1, wherein the fusion protein activates transcription of a target gene.

Clause 3. The fusion protein of clause 1 or 2, wherein the Cas protein comprises Cas9.

Clause 4. The fusion protein of clause 3, wherein the Cas9 comprises at least one amino acid mutation which knocks out nuclease activity of Cas9.

Clause 5. The fusion protein of clause 4, wherein the Cas protein comprises SEQ ID NO: 1 or SEQ ID NO: 10.

Clause 6. The fusion protein of any one of clauses 1-5, wherein the second polypeptide domain comprises a histone acetyltransferase effector domain.

Clause 7. The fusion protein of clause 6, wherein the histone acetyltransferase effector domain is a p300 histone acetyltransferase effector domain.

Clause 8. The fusion protein of any one of clauses 1-7, wherein the second polypeptide domain comprises SEQ ID NO: 2 or SEQ ID NO: 3.

Clause 9. The fusion protein of any one of clauses 1-8, wherein the first polypeptide domain comprises SEQ ID NO: 1 or SEQ ID NO: 10 and the second polypeptide domain comprises SEQ ID NO: 2 or SEQ ID NO. 3.

Clause 10. The fusion protein of any one of clauses 1-9, wherein the first polypeptide domain comprises SEQ ID NO: 1 and the second polypeptide domain comprises SEQ ID NO. 3, or the first polypeptide domain comprises SEQ ID NO: 10 and the second polypeptide domain comprises SEQ ID NO. 3.

Clause 11. The fusion protein of any one of clauses 1-10, further comprising a linker connecting the first polypeptide domain to the second polypeptide domain.

Clause 12. The fusion protein of any one of clauses 1-11, wherein the fusion protein comprises an amino acid sequence of SEQ ID NO: 140, 141, or 149.

Clause 13. A DNA targeting system comprising the fusion protein of any one of clauses 1-12 and at least one guide RNA (gRNA).

Clause 14. The DNA targeting system of clause 13, wherein the at least one gRNA comprises a 12-22 base pair complementary polynucleotide sequence of the target DNA sequence followed by a protospacer-adjacent motif.

Clause 15. The DNA targeting system of clause 13 or 14, wherein the at least one gRNA targets a target region, the target region comprises a target enhancer, target regulatory element, a cis-regulatory region of a target gene, or a trans-regulatory region of a target gene.

Clause 16. The DNA targeting system of clause 15, wherein the target region is a distal or proximal cis-regulatory region of the target gene.

Clause 17. The DNA targeting system of clause 15 or 16, wherein the target region is an enhancer region or a promoter region of the target gene.

Clause 18. The DNA targeting system of any one of clauses 15-17, wherein the target gene is an endogenous gene or a transgene.

Clause 19. The DNA targeting system of clause 15, wherein the target region comprises a target enhancer or a target regulatory element.

Clause 20. The DNA targeting system of clause 19, wherein the target enhancer or target regulatory element control the gene expression of more than one target gene.

Clause 21. The DNA targeting system of any one of clauses 15-20, wherein the DNA targeting system comprises between one and ten different gRNAs.

Clause 22. The DNA targeting system of any one of clauses 15-21, wherein the DNA targeting system comprises one gRNA.

Clause 23. The DNA targeting system of any one of clauses 15-22, wherein the target region is located on the same chromosome as the target gene.

Clause 24. The DNA targeting system of clause 23, wherein the target region is located about 1 base pair to about 100,000 base pairs upstream of a transcription start site of the target gene.

Clause 25. The DNA targeting system of clause 24, wherein the target region is located about 1000 base pairs to about 50,000 base pairs upstream of the transcription start site of the target gene.

Clause 26. The DNA targeting system of any one of clauses 15-22, wherein the target region is located on a different chromosome as the target gene.

Clause 27. The DNA targeting system of any one of clauses 15-28, wherein the different gRNAs bind to different target regions.

Clause 28. The DNA targeting system of clause 27, wherein the different gRNAs bind to target regions of different target genes.

Clause 29. The DNA targeting system of clause 27, wherein the expression of two or more target genes are activated.

Clause 30. The DNA targeting system of any one of clauses 15-29, wherein the target gene is selected from the group consisting of IL1RN, MYOD1, OCT4, HBE, HBG, HBD, HBB, MYOCD, PAX7, FGF1A, FGF1B, and FGF1C.

Clause 31. The DNA targeting system of clause 30, wherein the target region is at least one of HS2 enhancer of the human β-globin locus, distal regulatory region (DRR) of the MYOD gene, core enhancer (CE) of the MYOD gene, proximal (PE) enhancer region of the OCT4 gene, or distal (DE) enhancer region of the OCT4 gene.

Clause 32. The DNA targeting system of any one of clauses 13-31, wherein the gRNA comprises at least one of SEQ ID NOs: 23-73, 188-223, or 224-254.

Clause 33. An isolated polynucleotide encoding the fusion protein of any one of clauses 1-12 or the DNA targeting system of any one of clauses 13-32.

Clause 34. A vector comprising the isolated polynucleotide of clause 33.

Clause 35. A cell comprising the isolated polynucleotide of clause 33 or the vector of clause 34.

Clause 36. A kit comprising the fusion protein of any one of clauses 1-12, the DNA targeting system of clauses 13-32, the isolated polynucleotide of clause 33, the vector of clause 34, or the cell of clause 35.

Clause 37. A method of activating gene expression of a target gene in a cell, the method comprising contacting the cell with the fusion protein of any one of clauses 1-12, the DNA targeting system of clauses 13-32, the isolated polynucleotide of clause 33, or the vector of clause 34.

Clause 38. A method of activating gene expression of a target gene in a cell, the method comprising contacting the cell with a polynucleotide encoding a DNA targeting system, wherein the DNA targeting system comprises the fusion protein of any one of clauses 1-12 and at least one guide RNA (gRNA).

Clause 39. The method of clause 38, wherein the at least one gRNA comprises a 12-22 base pair complementary polynucleotide sequence of the target DNA sequence followed by a protospacer-adjacent motif.

Clause 40. The method of clause 38 or 39, wherein the at least one gRNA targets a target region, the target region is a cis-regulatory region or a trans-regulatory region of a target gene.

Clause 41. The method of clause 40, wherein the target region is a distal or proximal cis-regulatory region of the target gene.

Clause 42. The method of clause 40 or 41, wherein the target region is an enhancer region or a promoter region of the target gene.

Clause 43. The method of clause 40-42, wherein the target gene is an endogenous gene or a transgene.

Clause 44. The method of clause 43, wherein the DNA targeting system comprises between one and ten different gRNAs.

Clause 45. The method of clause 43, wherein the DNA targeting system comprises one gRNA.

Clause 46. The method of clause 40-45, wherein the target region is located on the same chromosome as the target gene.

Clause 47. The method of clause 46, wherein the target region is located about 1 base pair to about 100,000 base pairs upstream of a transcription start site of the target gene.

Clause 48. The method of clause 46, wherein the target region is located about 1000 base pairs to about 50,000 base pairs upstream of the transcription start site of the target gene.

Clause 49. The method of clause 40-45, wherein the target region is located on a different chromosome as the target gene.

Clause 50. The method of clause 40-45, wherein the different gRNAs bind to different target regions.

Clause 51. The method of clause 50, wherein the different gRNAs bind to target regions of different target genes.

Clause 52. The method of clause 51, wherein the expression of two or more target genes are activated.

Clause 53. The method of clause 40-52, wherein the target gene is selected from the group consisting of IL1RN, MYOD1, OCT4, HBE, HBG, HBD, HBB, MYOCD, PAX7, FGF1A, FGF1B, and FGF1C.

Clause 54. The method of clause 53, wherein the target region is at least one of HS2 enhancer of the human β-globin locus, distal regulatory region (DRR) of the MYOD gene, core enhancer (CE) of the MYOD gene, proximal (PE) enhancer region of the OCT4 gene, or distal (DE) enhancer region of the OCT4 gene.

Clause 55. The method of clause 37-54, wherein the gRNA comprises at least one of SEQ ID NOs: 23-73, 188-223, or 224-254.

Clause 56. The method of any one of clauses 37-55, wherein the DNA targeting system is delivered to the cell virally or non-virally.

Clause 57. The method of any one of clauses 37-56, wherein the cell is a mammalian cell.

Appendix-Sequences

Streptococcus pyogenes Cas 9 (with D10A, H849A) (SEQ ID NO: 1)
MDKKYSIGLAIGTNSVGWAVITDEYKVPSKKFKVLGNTDRHSIKKNLIGALLFDSGETA
EATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGY
AGYIDGGASQEEFYKFIKPILEKMDGTEELLVKLNREDLLRKQRTFDNGSIPHQIHLGEL
HAILRRQEDFYPFLKDNREKIEKILTFRIPYYVGPLARGNSRFAWMTRKSEETITPWNFEE
VVDKGASAQSFIERMTNFDKNLPNEKVLPKHSLLYEYFTVYNELTKVKYVTEGMRKPA
FLSGEQKKAIVDLLFKTNRKVTVKQLKEDYFKKIECFDSVEISGVEDRFNASLGTYHDLL
KIIKDKDFLDNEENEDILEDIVLTLTLFEDREMIEERLKTYAHLFDDKVMKQLKRRRYTG
WGRLSRKLINGIRDKQSGKTILDFLKSDGFANRNFMQLIHDDSLTFKEDIQKAQVSGQG
DSLHEHIANLAGSPAIKKGILQTVKVVDELVKVMGRHKPENIVIEMARENQTTQKGQKN
SRERMKRIEEGIKELGSQILKEHPVENTQLQNEKLYLYYLQNGRDMYVDQELDINRLSD
YDVDAIVPQSFLKDDSIDNKVLTRSDKNRGKSDNVPSEEVVKKMKNYWRQLLNAKLIT
QRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYG
DYKVYDVRKMIAKSEQEIGKATAKYFFYSNIMNFFKTEITLANGEIRKRPLIETNGETGEI
VWDKGRDFATVRKVLSMPQVNIVKKTEVQTGGFSKESILPKRNSDKLIARKKDWDPKK
YGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGS
PEDNEQKQLFVEQHKHYLDEIIEQISEFSKRVILADANLDKVLSAYNKHRDKPIREQAENI
IHLFTLTNLGAPAAFKYFDTTIDRKRYTSTKEVLDATLIHQSITGLYETRIDLSQLGGD
Human p300 (with L553M mutation) (SEQ ID NO: 2)
MAENVVEPGPPSAKRPKLSSPALSASASDGTDFGSLFDLEHDLPDELINSTELGLTNGGDI
NQLQTSLGMVQDAASKHKQLSELLRSGSSPNLNMGVGGPGQVMASQAQQSSPGLGLIN
SMVKSPMTQAGLTSPNMGMGTSGPNQGPTQSTGMMNSPVNQPAMGMNTGMNAGMN
PGMLAAGNGQGIMPNQVMNGSIGAGRGRQNMQYPNPGMGSAGNLLTEPLQQGSPQM
GGQTGLRGPQPLKMGMMNNPNPYGSPYTQNPGQQIGASGLGLQIQTKTVLSNNLSPFA
MDKKAVPGGGMPNMGQQPAPQVQQPGLVTPVAQGMGSGAHTADPEKRKLIQQQLVL
LLHAHKCQRREQANGEVRQCNLPHCRTMKNVLNHMTHCQSGKSCQVAHCASSRQIISH
WKNCTRHDCPVCLPLKNAGDKRNQQPILTGAPVGLGNPSSLGVGQQSAPNLSTVSQIDP
SSIERAYAALGLPYQVNQMPTQPQVQAKNQQNQQPGQSPQGMRPMSNMSASPMGVNG
GVGVQTPSLLSDSMLHSAINSQNPMMSENASVPSMGPMPTAAQPSTTGIRKQWHEDITQ
DLRNHLVHKLVQAIFPTPDPAALKDRRMENLVAYARKVEGDMYESANNRAEYYHLLA
EKIYKIQKELEEKRRTRLQKQNMLPNAAGMVPVSMNPGPNMGQPQPGMTSNGPLPDPS
MIRGSVPNQMMPRITPQSGLNQFGQMSMAQPPIVPRQTPPLQHHGQLAQPGALNPPMG
YGPRMQQPSNQGQFLPQTQFPSQGMNVTNIPLAPSSGQAPVSQAQMSSSSCPVNSPIMPP
GSQGSHIHCPQLPQPALHQNSPSPVPSRTPTPHHTPPSIGAQQPPATTIPAPVPTPPAMPPG
PQSQALHPPPRQTPTPPTTQLPQQVQPSLPAAPSADQPQQQPRSQQSTAASVPTPTAPLLP
PQPATPLSQPAVSIEGQVSNPPSTSSTEVNSQAIAEKQPSQEVKMEAKMEVDQPEPADTQ
PEDISESKVEDCKMESTETEERSTELKTEIKEEEDQPSTSATQSSPAPGQSKKKIFKPEELR
QALMPTLEALYRQDPESLPFRQPVDPQLLGIPDYFDIVKSPMDLSTIKRKLDTGQYQEPW
QYVDDIWLMFNNAWLYNRKTSRVYKYCSKLSEVFEQEIDPVMQSLGYCCGRKLEFSPQ
TLCCYGKQLCTIPRDATYYSYQNRYHFCEKCFNEIQGESVSLGDDPSQPQTTINKEQFSK
RKNDTLDPELFVECTECGRKMHQICVLHHEIIWPAGFVCDGCLKKSARTRKENKFSAKR
LPSTRLGTFLENRVNDFLRRQNHPESGEVTVRVVHASDKTVEVKPGMKARFVDSGEMA
ESFPYRTKALFAFEEIDGVDLCFFGMHVQEYGSDCPPPNQRRVYISYLDSVHFFRPKCLR
TAVYHEILIGYLEYVKKLGYTTGHIWACPPSEGDDYIFHCHPPDQKIPKPKRLQEWYKK
MLDKAVSERIVHDYKDIFKQATEDRLTSAKELPYFEGDFWPNVLEESIKELEQEEEERKR
EENTSNESTDVTKGDSKNAKKKNNKKTSKNKSSLSRGNKKKPGMPNVSNDLSQKLYAT
MEKHKEVFFVIRLIAGPAANSLPPIVDPDPLIPCDLMDGRDAFLTLARDKHLEFSSLRRA
QWSTMCMLVELHTQSQDRFVYTCNECKHHVETRWHCTVCEDYDLCITCYNTKNHDHK
MEKLGLGLDDESNNQQAAATQSPGDSRRLSIQRCIQSLVHACQCRNANCSLPSCQKMK
RVVQHTKGCKRKTNGGCPICKQLIALCCYHAKHCQENKCPVPFCLNIKQKLRQQQLQH
RLQQAQMLRRRMASMQRTGVVGQQQGLPSPTPATPTTPTGQQPTTPQTPQPTSQPQPTP
PNSMPPYLPRTQAAGPVSQGKAAGQVTPPTPPQTAQPPLPGPPPAAVEMAMQIQRAAET
QRQMAHVQIFQRPIQHQMPPMTPMAPMGMNPPPMTRGPSGHLEPGMGPTGMQQQPPW
SQGGLPQPQQLQSGMPRPAMMSVAQHGQPLNMAPQPGLGQVGISPLKPGTVSQQALQ
NLLRTLRSPSSPLQQQQVLSILHANPQLLAAFIKQRAAKYANSNPQPIPGQPGMPQGQPG
LQPPTMPGQQGVHSNPAMQNMNPMQAGVQRAGLPQQQPQQQLQPPMGGMSPQAQQ
MNMNHNTMPSQFRDILRRQQMMQQQQQQGAGPGIGPGMANHNQFQQPQGVGYPPQQ
QQRMQHHMQQMQQGNMGQIGQLPQALGAEAGASLQAYQQRLLQQQMGSPVQPNPM
SPQQHMLPNQAQSPHLQGQQIPNSLSNQVRSPQPVPSPRPQSQPPHSSPSPRMQPQPSPH
HVSPQTSSPHPGLVAAQANPMEQGHFASPDQNSMLSQLASNPGMANLHGASATDLGLS
TDNSDLNSNLSQSTLDIH
p300 Core Effector (aa 1048-1664 of SEQ ID NO: 2) (SEQ ID NO: 3)
IFKPEELRQALMPTLEALYRQDPESLPFRQPVDPQLLGIPDYFDIVKSPMDLSTIKRKLDT
GQYQEPWQYVDDIWLMFNNAWLYNRKTSRVYKYCSKLSEVFEQEIDPVMQSLGYCCG
RKLEFSPQTLCCYGKQLCTIPRDATYYSYQNRYHFCEKCFNEIQGESVSLGDDPSQPQTT
INKEQFSKRKNDTLDPELFVECTECGRKMHQICVLHHEIIWPAGFVCDGCLKKSARTRK
ENKFSAKRLPSTRLGTFLENRVNDFLRRQNHPESGEVTVRVVHASDKTVEVKPGMKAR
FVDSGEMAESFPYRTKALFAFEEIDGVDLCFFGMHVQEYGSDCPPPNQRRVYISYLDSV
HFFRPKCLRTAVYHEILIGYLEYVKKLGYTTGHIWACPPSEGDDYIFHCHPPDQKIPKPK
RLQEWYKKMLDKAVSERIVHDYKDIFKQATEDRLTSAKELPYFEGDFWPNVLEESIKEL
EQEEEERKREENTSNESTDVTKGDSKNAKKKNNKKTSKNKSSLSRGNKKKPGMPNVSN
DLSQKLYATMEKHKEVFFVIRLIAGPAANSLPPIVDPDPLIPCDLMDGRDAFLTLARDKH
LEFSSLRRAQWSTMCMLVELHTQSQD
p300 Core Effector (aa 1048-1664 of SEQ ID NO: 2 with D1399Y
mutation) (SEQ ID NO: 4)
IFKPEELRQALMPTLEALYRQDPESLPFRQPVDPQLLGIPDYFDIVKSPMDLSTIKRKLDT
GQYQEPWQYVDDIWLMFNNAWLYNRKTSRVYKYCSKLSEVFEQEIDPVMQSLGYCCG
RKLEFSPQTLCCYGKQLCTIPRDATYYSYQNRYHFCEKCFNEIQGESVSLGDDPSQPQTT
INKEQFSKRKNDTLDPELFVECTECGRKMHQICVLHHEIIWPAGFVCDGCLKKSARTRK
ENKFSAKRLPSTRLGTFLENRVNDFLRRQNHPESGEVTVRVVHASDKTVEVKPGMKAR
FVDSGEMAESFPYRTKALFAFEEIDGVDLCFFGMHVQEYGSDCPPPNQRRVYISYLYSV
HFFRPKCLRTAVYHEILIGYLEYVKKLGYTTGHIWACPPSEGDDYIFHCHPPDQKIPKPK
RLQEWYKKMLDKAVSERIVHDYKDIFKQATEDRLTSAKELPYFEGDFWPNVLEESIKEL
EQEEEERKREENTSNESTDVTKGDSKNAKKKNNKKTSKNKSSLSRGNKKKPGMPNVSN
DLSQKLYATMEKHKEVFFVIRLIAGPAANSLPPIVDPDPLIPCDLMDGRDAFLTLARDKH
LEFSSLRRAQWSTMCMLVELHTQSQD
p300 Core Effector (aa 1048-1664 of SEQ ID NO: 2 with 1645/1646
RR/EE mutations) (SEQ ID NO: 5)
IFKPEELRQALMPTLEALYRQDPESLPFRQPVDPQLLGIPDYFDIVKSPMDLSTIKRKLDT
GQYQEPWQYVDDIWLMFNNAWLYNRKTSRVYKYCSKLSEVFEQEIDPVMQSLGYCCG
RKLEFSPQTLCCYGKQLCTIPRDATYYSYQNRYHFCEKCFNEIQGESVSLGDDPSQPQTT
INKEQFSKRKNDTLDPELFVECTECGRKMHQICVLHHEIIWPAGFVCDGCLKKSARTRK
ENKFSAKRLPSTRLGTFLENRVNDFLRRQNHPESGEVTVRVVHASDKTVEVKPGMKAR
FVDSGEMAESFPYRTKALFAFEEIDGVDLCFFGMHVQEYGSDCPPPNQRRVYISYLDSV
HFFRPKCLRTAVYHEILIGYLEYVKKLGYTTGHIWACPPSEGDDYIFHCHPPDQKIPKPK
RLQEWYKKMLDKAVSERIVHDYKDIFKQATEDRLTSAKELPYFEGDFWPNVLEESIKEL
EQEEEERKREENTSNESTDVTKGDSKNAKKKNNKKTSKNKSSLSRGNKKKPGMPNVSN
DLSQKLYATMEKHKEVFFVIRLIAGPAANSLPPIVDPDPLIPCDLMDGRDAFLTLARDKH
LEFSSLEEAQWSTMCMLVELHTQSQD
p300 Core Effector (aa 1048-1664 of SEQ ID NO: 2 with C1204R
mutation) (SEQ ID NO: 6)
IFKPEELRQALMPTLEALYRQDPESLPFRQPVDPQLLGIPDYFDIVKSPMDLSTIKRKLDT
GQYQEPWQYVDDIWLMFNNAWLYNRKTSRVYKYCSKLSEVFEQEIDPVMQSLGYCCG
RKLEFSPQTLCCYGKQLCTIPRDATYYSYQNRYHFCEKRFNEIQGESVSLGDDPSQPQTT
INKEQFSKRKNDTLDPELFVECTECGRKMHQICVLHHEIIWPAGFVCDGCLKKSARTRK
ENKFSAKRLPSTRLGTFLENRVNDFLRRQNHPESGEVTVRVVHASDKTVEVKPGMKAR
FVDSGEMAESFPYRTKALFAFEEIDGVDLCFFGMHVQEYGSDCPPPNQRRVYISYLDSV
HFFRPKCLRTAVYHEILIGYLEYVKKLGYTTGHIWACPPSEGDDYIFHCHPPDQKIPKPK
RLQEWYKKMLDKAVSERIVHDYKDIFKQATEDRLTSAKELPYFEGDFWPNVLEESIKEL
EQEEEERKREENTSNESTDVTKGDSKNAKKKNNKKTSKNKSSLSRGNKKKPGMPNVSN
DLSQKLYATMEKHKEVFFVIRLIAGPAANSLPPIVDPDPLIPCDLMDGRDAFLTLARDKH
LEFSSLRRAQWSTMCMLVELHTQSQD
p300 Core Effector (aa 1048-1664 of SEQ ID NO: 2 with Y1467F
mutation) (SEQ ID NO: 7)
IFKPEELRQALMPTLEALYRQDPESLPFRQPVDPQLLGIPDYFDIVKSPMDLSTIKRKLDT
GQYQEPWQYVDDIWLMFNNAWLYNRKTSRVYKYCSKLSEVFEQEIDPVMQSLGYCCG
RKLEFSPQTLCCYGKQLCTIPRDATYYSYQNRYHFCEKCFNEIQGESVSLGDDPSQPQTT
INKEQFSKRKNDTLDPELFVECTECGRKMHQICVLHHEIIWPAGFVCDGCLKKSARTRK
ENKFSAKRLPSTRLGTFLENRVNDFLRRQNHPESGEVTVRVVHASDKTVEVKPGMKAR
FVDSGEMAESFPYRTKALFAFEEIDGVDLCFFGMHVQEYGSDCPPPNQRRVYISYLDSV
HFFRPKCLRTAVYHEILIGYLEYVKKLGYTTGHIWACPPSEGDDYIFHCHPPDQKIPKPK
RLQEWFKKMLDKAVSERIVHDYKDIFKQATEDRLTSAKELPYFEGDFWPNVLEESIKEL
EQEEEERKREENTSNESTDVTKGDSKNAKKKNNKKTSKNKSSLSRGNKKKPGMPNVSN
DLSQKLYATMEKHKEVFFVIRLIAGPAANSLPPIVDPDPLIPCDLMDGRDAFLTLARDKH
LEFSSLRRAQWSTMCMLVELHTQSQD
p300 Core Effector (aa 1048-1664 of SEQ ID NO: 2 with 1396/1397
SY/WW mutations) (SEQ ID NO: 8)
IFKPEELRQALMPTLEALYRQDPESLPFRQPVDPQLLGIPDYFDIVKSPMDLSTIKRKLDT
GQYQEPWQYVDDIWLMFNNAWLYNRKTSRVYKYCSKLSEVFEQEIDPVMQSLGYCCG
RKLEFSPQTLCCYGKQLCTIPRDATYYSYQNRYHFCEKCFNEIQGESVSLGDDPSQPQTT
INKEQFSKRKNDTLDPELFVECTECGRKMHQICVLHHEIIWPAGFVCDGCLKKSARTRK
ENKFSAKRLPSTRLGTFLENRVNDFLRRQNHPESGEVTVRVVHASDKTVEVKPGMKAR
FVDSGEMAESFPYRTKALFAFEEIDGVDLCFFGMHVQEYGSDCPPPNQRRVYIWWLDS
VHFFRPKCLRTAVYHEILIGYLEYVKKLGYTTGHIWACPPSEGDDYIFHCHPPDQKIPKP
KRLQEWYKKMLDKAVSERIVHDYKDIFKQATEDRLTSAKELPYFEGDFWPNVLEESIKE
LEQEEEERKREENTSNESTDVTKGDSKNAKKKNNKKTSKNKSSLSRGNKKKPGMPNVS
NDLSQKLYATMEKHKEVFFVIRLIAGPAANSLPPIVDPDPLIPCDLMDGRDAFLTLARDK
HLEFSSLRRAQWSTMCMLVELHTQSQD
p300 Core Effector (aa 1048-1664 of SEQ ID NO: 2 with H1415A,
E1423A, Y1424A, L14285, Y1430A, and H1434A mutations)
(SEQ ID NO: 9)
IFKPEELRQALMPTLEALYRQDPESLPFRQPVDPQLLGIPDYFDIVKSPMDLSTIKRKLDT
GQYQEPWQYVDDIWLMFNNAWLYNRKTSRVYKYCSKLSEVFEQEIDPVMQSLGYCCG
RKLEFSPQTLCCYGKQLCTIPRDATYYSYQNRYHFCEKCFNEIQGESVSLGDDPSQPQTT
INKEQFSKRKNDTLDPELFVECTECGRKMHQICVLHHEIIWPAGFVCDGCLKKSARTRK
ENKFSAKRLPSTRLGTFLENRVNDFLRRQNHPESGEVTVRVVHASDKTVEVKPGMKAR
FVDSGEMAESFPYRTKALFAFEEIDGVDLCFFGMHVQEYGSDCPPPNQRRVYISYLDSV
HFFRPKCLRTAVYAEILIGYLAAVKKSGATTGAIWACPPSEGDDYIFHCHPPDQKIPKPK
RLQEWYKKMLDKAVSERIVHDYKDIFKQATEDRLTSAKELPYFEGDFWPNVLEESIKEL
EQEEEERKREENTSNESTDVTKGDSKNAKKKNNKKTSKNKSSLSRGNKKKPGMPNVSN
DLSQKLYATMEKHKEVFFVIRLIAGPAANSLPPIVDPDPLIPCDLMDGRDAFLTLARDKH
LEFSSLRRAQWSTMCMLVELHTQSQD
Neisseria meningitidis Cas9 (with D16A, D587A, H588A, and N611A
mutations) (SEQ ID NO: 10)
MAAFKPNPINYILGLAIGIASVGWAMVEIDEDENPICLIDLGVRVFERAEVPKTGDSLAM
ARRLARSVRRLTRRRAHRLLRARRLLKREGVLQAADFDENGLIKSLPNTPWQLRAAAL
DRKLTPLEWSAVLLHLIKHRGYLSQRKNEGETADKELGALLKGVADNAHALQTGDFRT
PAELALNKFEKESGHIRNQRGDYSHTFSRKDLQAELILLFEKQKEFGNPHVSGGLKEGIE
TLLMTQRPALSGDAVQKMLGHCTFEPAEPKAAKNTYTAERFIWLTKLNNLRILEQGSER
PLTDTERATLMDEPYRKSKLTYAQARKLLGLEDTAFFKGLRYGKDNAEASTLMEMKA
YHAISRALEKEGLKDKKSPLNLSPELQDEIGTAFSLFKTDEDITGRLKDRIQPEILEALLKH
ISFDKFVQISLKALRRIVPLMEQGKRYDEACAEIYGDHYGKKNTEEKIYLPPIPADEIRNP
VVLRALSQARKVINGVVRRYGSPARIHIETAREVGKSFKDRKEIEKRQEENRKDREKAA
AKFREYFPNFVGEPKSKDILKLRLYEQQHGKCLYSGKEINLGRLNEKGYVEIAAALPFSR
TWDDSFNNKVLVLGSEAQNKGNQTPYEYFNGKDNSREWQEFKARVETSRFPRSKKQRI
LLQKFDEDGFKERNLNDTRYVNRFLCQFVADRMRLTGKGKKRVFASNGQITNLLRGFW
GLRKVRAENDRHHALDAVVVACSTVAMQQKITRFVRYKEMNAFDGKTIDKETGEVLH
QKTHFPQPWEFFAQEVMIRVFGKPDGKPEFEEADTPEKLRTLLAEKLSSRPEAVHEYVTP
LFVSRAPNRKMSGQGHMETVKSAKRLDEGVSVLRVPLTQLKLKDLEKMVNREREPKL
YEALKARLEAHKDDPAKAFAEPFYKYDKAGNRTQQVKAVRVEQVQKTGVWVRNHNG
IADNATMVRVDVFEKGDKYYLVPIYSWQVAKGILPDRAVVQGKDEEDWQLIDDSFNFK
FSLHPNDLVEVITKKARMFGYFASCHRGTGNINIRIHDLDHKIGKNGILEGIGVKTALSFQ
KYQIDELGKEIRPCRLKKRPPVR
3X “Flag” Epitope (SEQ ID NO: 11)
DYKDHDGDYKDHDIDYKDDDDK
Nuclear Localization Sequence (SEQ ID NO: 12)
PKKKRKVG
HA Epitope (SEQ ID NO: 13)
YPYDVPDYAS
VP64 Effector (SEQ ID NO: 14)
DALDDFDLDMLGSDALDDFDLDMLGSDALDDFDLDMLGSDALDDFDLDML

1-57. (canceled)

58. A DNA targeting system comprising:
(1) a fusion protein comprising a first polypeptide domain and a second polypeptide domain, wherein the first polypeptide domain comprises a Clustered Regularly Interspaced Short Palindromic Repeats associated (Cas) protein, wherein the second polypeptide domain comprises a p300 histone acetyltransferase effector domain, and wherein the fusion protein activates transcription of a target gene; and
(2) at least one guide RNA (gRNA).

59. The DNA targeting system of claim 58, wherein the first polypeptide domain comprises the sequence of SEQ ID NO: 1 or SEQ ID NO: 10.

60. The DNA targeting system of claim 58, wherein the second polypeptide domain comprises the sequence of SEQ ID NO: 2 or SEQ ID NO: 3.

61. The DNA targeting system of claim 58, wherein the first polypeptide domain comprises the polypeptide sequence of SEQ ID NO: 10 and the second polypeptide domain comprises the polypeptide sequence of SEQ ID NO: 3, or the first polypeptide domain comprises the polypeptide sequence of SEQ ID NO: 1 and the second polypeptide domain comprises the polypeptide sequence of SEQ ID NO: 2.

62. The DNA targeting system of claim 58, wherein the fusion protein further comprises a linker connecting the first polypeptide domain to the second polypeptide domain

63. The DNA targeting system of claim 58, wherein the fusion protein comprises the polypeptide sequence of SEQ ID NO: 140, SEQ ID NO: 141, or SEQ ID NO: 149.

64. The DNA targeting system of claim 58, wherein the at least one gRNA targets a target region of the target gene.

65. The DNA targeting system of claim 64, wherein the target region comprises a target enhancer, target regulatory element, a cis-regulatory region of a target gene, or a trans-regulatory region of the target gene.

66. The DNA targeting system of claim 64, wherein the target region is a distal or proximal cis-regulatory region of the target gene.

67. The DNA targeting system of claim 64, wherein the target region is a promoter region of the target gene.

68. The DNA targeting system of claim 64, wherein the target region is located on the same chromosome as the target gene.

69. The DNA targeting system of claim 64, wherein the target region is located about 1 base pair to about 100,000 base pairs upstream of a transcription start site of the target gene.

70. The DNA targeting system of claim 64, wherein the target region is located on a different chromosome as the target gene.

71. The DNA targeting system of claim 58, wherein the target gene is selected from the group consisting of IL1RN, MYOD1, OCT4, HBE, HBG, HBD, HBB, MYOCD, PAX7, FGF1A, FGF1B, and FGF1C.

72. The DNA targeting system of claim 58, wherein the target region is at least one of HS2 enhancer of the human β-globin locus, distal regulatory region (DRR) of the MYOD gene, core enhancer (CE) of the MYOD gene, proximal (PE) enhancer region of the OCT4 gene, or distal (DE) enhancer region of the OCT4 gene.

73. The DNA targeting system of claim 58, wherein the gRNA comprises a polynucleotide encoded by a sequence selected from SEQ ID NO: 23-27.

74. A method of activating gene expression of a target gene in an isolated cell, the method comprising contacting the isolated cell with one or more polynucleotides encoding the DNA targeting system of claim 58.

75. The method of claim 74, wherein the DNA targeting system is delivered to the isolated cell virally.

76. The method of claim 74, wherein the DNA targeting system is delivered to the isolated cell non-virally.

77. The method of claim 74, wherein the isolated cell is a mammalian cell.


SEQUENCE LISTING (278 sequences)

SEQ ID NO: 1 - protein, 1368 aa, Streptococcus pyogenes
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 2 - protein, 2414 aa, Homo sapiens
MAENVVEPGPPSAKRPKLSSPALSASASDGTDFGSLFDLEHDLPDELINSTELGLTNGGDINQLQTSLGMVQDAASKHKQLSELLRSGSSPNLNMGVGGPGQVMASQAQQSSPGLGLINSMVKSPMTQAGLTSPNMGMGTSGPNQGPTQSTGMMNSPVNQPAMGMNTGMNAGMNPGMLAAGNGQGIMPNQVMNGSIGAGRGRQNMQYPNPGMGSAGNLLTEPLQQGSPQMGGQTGLRGPQPLKMGMMNNPNPYGSPYTQNPGQQIGASGLGLQIQTKTVLSNNLSPFAMDKKAVPGGGMPNMGQQPAPQVQQPGLVTPVAQGMGSGAHTADPEKRKLIQQQLVLLLHAHKCQRREQANGEVRQCNLPHCRTMKNVLNHMTHCQSGKSCQVAHCASSRQIISHWKNCTRHDCPVCLPLKNAGDKRNQQPILTGAPVGLGNPSSLGVGQQSAPNLSTVSQIDPSSIERAYAALGLPYQVNQMPTQPQVQAKNQQNQQPGQSPQGMRPMSNMSASPMGVNGGVGVQTPSLLSDSMLHSAINSQNPMMSENASVPSMGPMPTAAQPSTTGIRKQWHEDITQDLRNHLVHKLVQAIFPTPDPAALKDRRMENLVAYARKVEGDMYESANNRAEYYHLLAEKIYKIQKELEEKRRTRLQKQNMLPNAAGMVPVSMNPGPNMGQPQPGMTSNGPLPDPSMIRGSVPNQMMPRITPQSGLNQFGQMSMAQPPIVPRQTPPLQHHGQLAQPGALNPPMGYGPRMQQPSNQGQFLPQTQFPSQGMNVTNIPLAPSSGQAPVSQAQMSSSSCPVNSPIMPPGSQGSHIHCPQLPQPALHQNSPSPVPSRTPTPHHTPPSIGAQQPPATTIPAPVPTPPAMPPGPQSQALHPPPRQTPTPPTTQLPQQVQPSLPAAPSADQPQQQPRSQQSTAASVPTPTAPLLPPQPATPLSQPAVSIEGQVSNPPSTSSTEVNSQAIAEKQPSQEVKMEAKMEVDQPEPADTQPEDISESKVEDCKMESTETEERSTELKTEIKEEEDQPSTSATQSSPAPGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQAAGPVSQGKAAGQVTPPTPPQTAQPPLPGPPPAAVEMAMQIQRAAETQRQMAHVQIFQRPIQHQMPPMTPMAPMGMNPPPMTRGPSGHLEPGMGPTGMQQQPPWSQGGLPQPQQLQSGMPRPAMMSVAQHGQPLNMAPQPGLGQVGISPLKPGTVSQQALQNLLRTLRSPSSPLQQQQVLSILHANPQLLAAFIKQRAAKYANSNPQPIPGQPGMPQGQPGLQPPTMPGQQGVHSNPAMQNMNPMQAGVQRAGLPQQQPQQQLQPPMGGMSPQAQQMNMNHNTMPSQFRDILRRQQMMQQQQQQGAGPGIGPGMANHNQFQQPQGVGYPPQQQQRMQHHMQQMQQGNMGQIGQLPQALGAEAGASLQAYQQRLLQQQMGSPVQPNPMSPQQHMLPNQAQSPHLQGQQIPNSLSNQVRSPQPVPSPRPQSQPPHSSPSPRMQPQPSPHHVSPQTSSPHPGLVAAQANPMEQGHFASPDQNSMLSQLASNPGMANLHGASATDLGLSTDNSDLNSNLSQSTLDIH

SEQ ID NO: 3 - protein, 617 aa, Homo sapiens
IFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 4 - protein, 617 aa, Homo sapiens
IFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLYSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 5 - protein, 617 aa, Homo sapiens
IFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLEEAQWSTMCMLVELHTQSQD

SEQ ID NO: 6 - protein, 617 aa, Homo sapiens
IFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKRFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 7 - protein, 617 aa, Homo sapiens
IFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWFKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 8 - protein, 617 aa, Homo sapiens
IFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYIWWLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 9 - protein, 617 aa, Homo sapiens
IFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYAEILIGYLAAVKKSGATTGAIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 10 - protein, 1082 aa, Neisseria meningitidis
MAAFKPNPINYILGLAIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIAAALPFSRTWDDSFNNKVLVLGSEAQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 11 - protein, 22 aa, synthetic construct
  REGION 1..22: note = Description of Artificial Sequence: Synthetic peptide
DYKDHDGDYKDHDIDYKDDDDK

SEQ ID NO: 12 - protein, 8 aa, synthetic construct
  REGION 1..8: note = Description of Artificial Sequence: Synthetic peptide
PKKKRKVG

SEQ ID NO: 13 - protein, 10 aa, synthetic construct
  REGION 1..10: note = Description of Artificial Sequence: Synthetic peptide
YPYDVPDYAS

SEQ ID NO: 14 - protein, 50 aa, synthetic construct
  REGION 1..50: note = Description of Artificial Sequence: Synthetic polypeptide
DALDDFDLDMLGSDALDDFDLDMLGSDALDDFDLDMLGSDALDDFDLDML

SEQ ID NO: 15 - DNA, 17 bp, synthetic construct
  misc_feature 1..17: note = Description of Artificial Sequence: Synthetic oligonucleotide
  source 1..17: mol_type = other DNA
gggctcctccttgtact

SEQ ID NO: 16 - DNA, 18 bp, synthetic construct
  misc_feature 1..18: note = Description of Artificial Sequence: Synthetic oligonucleotide
  source 1..18: mol_type = other DNA
acgcagataagaaccagt

SEQ ID NO: 17 - DNA, 17 bp, synthetic construct
  misc_feature 1..17: note = Description of Artificial Sequence: Synthetic oligonucleotide
  source 1..17: mol_type = other DNA
ggcatcaagtcagccat

SEQ ID NO: 18 - DNA, 18 bp, synthetic construct
  misc_feature 1..18: note = Description of Artificial Sequence: Synthetic oligonucleotide
  source 1..18: mol_type = other DNA
agcctgagtcaccctcct

SEQ ID NO: 19 - DNA, 17 bp, synthetic construct
  misc_feature 1..17: note = Description of Artificial Sequence: Synthetic oligonucleotide
  source 1..17: mol_type = other DNA
gggctcctccttgtact

SEQ ID NO: 20 - DNA, 18 bp, synthetic construct
  misc_feature 1..18: note = Description of Artificial Sequence: Synthetic oligonucleotide
  source 1..18: mol_type = other DNA
acgcagataagaaccagt

SEQ ID NO: 21 - DNA, 17 bp, synthetic construct
  misc_feature 1..17: note = Description of Artificial Sequence: Synthetic oligonucleotide
  source 1..17: mol_type = other DNA
ggcatcaagtcagccat

SEQ ID NO: 22 - DNA, 18 bp, synthetic construct
  misc_feature 1..18: note = Description of Artificial Sequence: Synthetic oligonucleotide
  source 1..18: mol_type = other DNA
agcctgagtcaccctcct

SEQ ID NO: 23 - DNA, 19 bp, synthetic construct
  misc_feature 1..19: note = Description of Artificial Sequence: Synthetic oligonucleotide
  source 1..19: mol_type = other DNA
tgtactctctgaggtgctc

SEQ ID NO: 24 - DNA, 19 bp, synthetic construct
  misc_feature 1..19: note = Description of Artificial Sequence: Synthetic oligonucleotide
  source 1..19: mol_type = other DNA
acgcagataagaaccagtt

SEQ ID NO: 25 - DNA, 19 bp, synthetic construct
  misc_feature 1..19: note = Description of Artificial Sequence: Synthetic oligonucleotide
  source 1..19: mol_type = other DNA
catcaagtcagccatcagc

SEQ ID NO: 26 - DNA, 19 bp, synthetic construct
  misc_feature 1..19: note = Description of Artificial Sequence: Synthetic oligonucleotide
  source 1..19: mol_type = other DNA
gagtcaccctcctggaaac

SEQ ID NO: 27 - DNA, 19 bp, synthetic construct
  misc_feature 1..19: note = Description of Artificial Sequence: Synthetic oligonucleotide
  source 1..19: mol_type = other DNA
cctgggctccggggcgttt

SEQ ID NO: 28 - DNA, 19 bp, synthetic construct
  misc_feature 1..19: note = Description of Artificial Sequence: Synthetic oligonucleotide
  source 1..19: mol_type = other DNA
ggcccctgcggccaccccg

SEQ ID NO: 29 - DNA, 19 bp, synthetic construct
  misc_feature 1..19: note = Description of Artificial Sequence: Synthetic oligonucleotide
  source 1..19: mol_type = other DNA
ctccctccctgcccggtag

SEQ ID NO: 30 - DNA, 19 bp, synthetic construct
  misc_feature 1..19: note = Description of Artificial Sequence: Synthetic oligonucleotide
  source 1..19: mol_type = other DNA
aggtttggaaagggcgtgc

SEQ ID NO: 31 - DNA, 20 bp, synthetic construct
  misc_feature 1..20: note = Description of Artificial Sequence: Synthetic oligonucleotide
  source 1..20: mol_type = other DNA
actccactgcactccagtct

SEQ ID NO: 32 - DNA, 20 bp, synthetic construct
  misc_feature 1..20: note = Description of Artificial Sequence: Synthetic oligonucleotide
  source 1..20: mol_type = other DNA
tctgtgggggacctgcactg

SEQ ID NO: 33 - DNA, 20 bp, synthetic construct
  misc_feature 1..20: note = Description of Artificial Sequence: Synthetic oligonucleotide
  source 1..20: mol_type = other DNA
ggggcgccagttgtgtctcc

SEQ ID NO: 34 - DNA, 20 bp, synthetic construct
  misc_feature 1..20: note = Description of Artificial Sequence: Synthetic oligonucleotide
  source 1..20: mol_type = other DNA
acaccattgccaccaccatt

SEQ ID NO: 35 - DNA, 19 bp, synthetic construct
  misc_feature 1..19: note = Description of Artificial Sequence: Synthetic oligonucleotide
  source 1..19: mol_type = other DNA
tgttttcagcttccaaact

SEQ ID NO: 36 - DNA, 19 bp, synthetic construct
  misc_feature 1..19: note = Description of Artificial Sequence: Synthetic oligonucleotide
  source 1..19: mol_type = other DNA
catgaagacagcagaagcc

SEQ ID NO: 37 - DNA, 19 bp, synthetic construct
  misc_feature 1..19: note = Description of Artificial Sequence: Synthetic oligonucleotide
  source 1..19: mol_type = other DNA
ggcccacattcctttccag

SEQ ID NO: 38 - DNA, 19 bp, synthetic construct
  misc_feature 1..19: note = Description of Artificial Sequence: Synthetic oligonucleotide
  source 1..19: mol_type = other DNA
ggctggattgggtttccag

SEQ ID NO: 39 - DNA, 19 bp, synthetic construct
  misc_feature 1..19: note = Description of Artificial Sequence: Synthetic oligonucleotide
  source 1..19: mol_type = other DNA
caactgagtcctgaggttt

SEQ ID NO: 40 - DNA, 19 bp, synthetic construct
  misc_feature 1..19: note = Description of Artificial Sequence: Synthetic oligonucleotide
  source 1..19: mol_type = other DNA
ctcacagcacagccagtgt

SEQ ID NO: 41 - DNA, 19 bp, synthetic construct
  misc_feature 1..19: note = Description of Artificial Sequence: Synthetic oligonucleotide
  source 1..19: mol_type = other DNA
cagcagctggtcacaaagc

SEQ ID NO: 42 - DNA, 19 bp, synthetic construct
  misc_feature 1..19: note = Description of Artificial Sequence: Synthetic oligonucleotide
  source 1..19: mol_type = other DNA
cttcctataaacttctgag

SEQ ID NO: 43 - DNA, 20 bp, synthetic construct
  misc_feature 1..20: note = Description of Artificial Sequence: Synthetic oligonucleotide
  source 1..20: mol_type = other DNA
agtgataagacacccgcttt

SEQ ID NO: 44 - DNA, 20 bp, synthetic construct
  misc_feature 1..20: note = Description of Artificial Sequence: Synthetic oligonucleotide
  source 1..20: mol_type = other DNA
cagacatctaataccacggt

SEQ ID NO: 45 - DNA, 20 bp, synthetic construct
  misc_feature 1..20: note = Description of Artificial Sequence: Synthetic oligonucleotide
  source 1..20: mol_type = other DNA
agggagaacggggcctaccg

SEQ ID NO: 46 - DNA, 20 bp, synthetic construct
  misc_feature 1..20: note = Description of Artificial Sequence: Synthetic oligonucleotide
  source 1..20: mol_type = other DNA
acttcaggttcaaagaagcc

SEQ ID NO: 47 - DNA, 20 bp, synthetic construct
  misc_feature 1..20: note = Description of Artificial Sequence: Synthetic oligonucleotide
  source 1..20: mol_type = other DNA
ttttccccacccagggccta

SEQ ID NO: 48 - DNA, 20 bp, synthetic construct
  misc_feature 1..20: note = Description of Artificial Sequence: Synthetic oligonucleotide
  source 1..20: mol_type = other DNA
ccctgggtggggaaaaccag

SEQ ID NO: 49 - DNA, 20 bp, synthetic construct
  misc_feature 1..20: note = Description of Artificial Sequence: Synthetic oligonucleotide
  source 1..20: mol_type = other DNA
ggaggaacatgcttcggaac

SEQ ID NO: 50 - DNA, 20 bp, synthetic construct
  misc_feature 1..20: note = Description of Artificial Sequence: Synthetic oligonucleotide
  source 1..20: mol_type = other DNA
gtgccgtgatggttctgtcc

SEQ ID NO: 51 - DNA, 20 bp, synthetic construct
  misc_feature 1..20: note = Description of Artificial Sequence: Synthetic oligonucleotide
  source 1..20: mol_type = other DNA
ggtctgccggaaggtctaca

SEQ ID NO: 52 - DNA, 20 bp, synthetic construct
  misc_feature 1..20: note = Description of Artificial Sequence: Synthetic oligonucleotide
  source 1..20: mol_type = other DNA
tcggcctttaactgcccaaa

SEQ ID NO: 53 - DNA, 20 bp, synthetic construct
  misc_feature 1..20: note = Description of Artificial Sequence: Synthetic oligonucleotide
  source 1..20: mol_type = other DNA
gcatgacaaaggtgccgtga

SEQ ID NO: 54 - DNA, 20 bp, synthetic construct
  misc_feature 1..20: note = Description of Artificial Sequence: Synthetic oligonucleotide
  source 1..20: mol_type = other DNA
cctgccttttgggcagttaa

SEQ ID NO: 55 - DNA, 20 bp, synthetic construct
  misc_feature 1..20: note = Description of Artificial Sequence: Synthetic oligonucleotide
  source 1..20: mol_type = other DNA
aatatgtcacattctgtctc

SEQ ID NO: 56 - DNA, 20 bp, synthetic construct
  misc_feature 1..20: note = Description of Artificial Sequence: Synthetic oligonucleotide
  source 1..20: mol_type = other DNA
ggactatgggaggtcactaa

SEQ ID NO: 57 - DNA, 20 bp, synthetic construct
  misc_feature 1..20: note = Description of Artificial Sequence: Synthetic oligonucleotide
  source 1..20: mol_type = other DNA
gaaggttacacagaaccaga

SEQ ID NO: 58 - DNA, 20 bp, synthetic construct
  misc_feature 1..20: note = Description of Artificial Sequence: Synthetic oligonucleotide
  source 1..20: mol_type = other DNA
gccctgtaagcatcctgctg

SEQ ID NO: 59 - DNA, 20 bp, synthetic construct
  misc_feature 1..20: note = Description of Artificial Sequence: Synthetic oligonucleotide
  source 1..20: mol_type = other DNA
ccactgctaactgaaagaga

SEQ ID NO: 60 - DNA, 20 bp, synthetic construct
  misc_feature 1..20: note = Description of Artificial Sequence: Synthetic oligonucleotide
  source 1..20: mol_type = other DNA
agccacagtttcagcgcagt

SEQ ID NO: 61 - DNA, 20 bp, synthetic construct
  misc_feature 1..20: note = Description of Artificial Sequence: Synthetic oligonucleotide
  source 1..20: mol_type = other DNA
ctgtttcatcttagaaaaat

SEQ ID NO: 62 - DNA, 20 bp, synthetic construct
  misc_feature 1..20: note = Description of Artificial Sequence: Synthetic oligonucleotide
  source 1..20: mol_type = other DNA
gaatgttctttggcaggtac

SEQ ID NO: 63 - DNA, 20 bp, synthetic construct
  misc_feature 1..20: note = Description of Artificial Sequence: Synthetic oligonucleotide
  source 1..20: mol_type = other DNA
cgcacatcttatgtcttaga

SEQ ID NO: 64 - DNA, 20 bp, synthetic construct
  misc_feature 1..20: note = Description of Artificial Sequence: Synthetic oligonucleotide
  source 1..20: mol_type = other DNA
cttaagagagctagaactgg

SEQ ID NO: 65 - DNA, 20 bp, synthetic construct
  misc_feature 1..20: note = Description of Artificial Sequence: Synthetic oligonucleotide
  source 1..20: mol_type = other DNA
tcccaaagtacagtaccttg

SEQ ID NO: 66 - DNA, 20 bp, synthetic construct
  misc_feature 1..20: note = Description of Artificial Sequence: Synthetic oligonucleotide
  source 1..20: mol_type = other DNA
tccctagagaggacagacag

SEQ ID NO: 67 - DNA, 20 bp, synthetic construct
  misc_feature 1..20: note = Description of Artificial Sequence: Synthetic oligonucleotide
  source 1..20: mol_type = other DNA
tcatagagaaatgaaaagag

SEQ ID NO: 68 - DNA, 20 bp, synthetic construct
  misc_feature 1..20: note = Description of Artificial Sequence: Synthetic oligonucleotide
  source 1..20: mol_type = other DNA
ataatataccctgactccta

SEQ ID NO: 69 - DNA, 20 bp, synthetic construct
  misc_feature 1..20: note = Description of Artificial Sequence: Synthetic oligonucleotide
  source 1..20: mol_type = other DNA
aggccacctgcaagataaat

SEQ ID NO: 70 - DNA, 20 bp, synthetic construct
  misc_feature 1..20: note = Description of Artificial Sequence: Synthetic oligonucleotide
  source 1..20: mol_type = other DNA
tgttgttatcaattgccata

SEQ ID NO: 71 - DNA, 20 bp, synthetic construct
  misc_feature 1..20: note = Description of Artificial Sequence: Synthetic oligonucleotide
  source 1..20: mol_type = other DNA
atcccttccagcatcctcat

SEQ ID NO: 72 - DNA, 20 bp, synthetic construct
  misc_feature 1..20: note = Description of Artificial Sequence: Synthetic oligonucleotide
  source 1..20: mol_type = other DNA
gtgcttcaaaaccatttgct

SEQ ID NO: 73 - DNA, 20 bp, synthetic construct
  misc_feature 1..20: note = Description of Artificial Sequence: Synthetic oligonucleotide
  source 1..20: mol_type = other DNA
gatacatgttttattcttat

SEQ ID NO: 74 - DNA, 20 bp, synthetic construct
  misc_feature 1..20: note = Description of Artificial Sequence: Synthetic primer
  source 1..20: mol_type = other DNA
caatgaccccttcattgacc

SEQ ID NO: 75 - DNA, 20 bp, synthetic construct
  misc_feature 1..20: note = Description of Artificial Sequence: Synthetic primer
  source 1..20: mol_type = other DNA
ttgattttggagggatctcg

SEQ ID NO: 76 - DNA, 20 bp, synthetic construct
  misc_feature 1..20: note = Description of Artificial Sequence: Synthetic primer
  source 1..20: mol_type = other DNA
ggaatccatggagggaagat

SEQ ID NO: 77 - DNA, 20 bp, synthetic construct
  misc_feature 1..20: note = Description of Artificial Sequence: Synthetic primer
  source 1..20: mol_type = other DNA
tgttctcgctcaggtcagtg

SEQ ID NO: 78 - DNA, 20 bp, synthetic construct
  misc_feature 1..20: note = Description of Artificial Sequence: Synthetic primer
  source 1..20: mol_type = other DNA
tccctctttcacggtctcac

SEQ ID NO: 79 - DNA, 20 bp, synthetic construct
  misc_feature 1..20: note = Description of Artificial Sequence: Synthetic primer
  source 1..20: mol_type = other DNA
aacacccgactgctgtatcc

SEQ ID NO: 80 - DNA, 30 bp, synthetic construct
  misc_feature 1..30: note = Description of Artificial Sequence: Synthetic primer
  source 1..30: mol_type = other DNA
cgaaagagaaagcgaaccagtatcgagaac

SEQ ID NO: 81 - DNA, 27 bp, synthetic construct
  misc_feature 1..27: note = Description of Artificial Sequence: Synthetic primer
  source 1..27: mol_type = other DNA
cgttgtgcatagtcgctgcttgatcgc

SEQ ID NO: 82 - DNA, 20 bp, synthetic construct
  misc_feature 1..20: note = Description of Artificial Sequence: Synthetic primer
  source 1..20: mol_type = other DNA
gcacgtggatcctgagaact

SEQ ID NO: 83 - DNA, 21 bp, synthetic construct
  misc_feature 1..21: note = Description of Artificial Sequence: Synthetic primer
  source 1..21: mol_type = other DNA
attggacagcaagaaagcgag

SEQ ID NO: 84 - DNA, 20 bp, synthetic construct
  misc_feature 1..20: note = Description of Artificial Sequence: Synthetic primer
  source 1..20: mol_type = other DNA
gcacgtggatcctgagaact

SEQ ID NO: 85 - DNA, 22 bp, synthetic construct
  misc_feature 1..22: note = Description of Artificial Sequence: Synthetic primer
  source 1..22: mol_type = other DNA
caggaaacagtccaggatctca

SEQ ID NO: 86 - DNA, 21 bp, synthetic construct
  misc_feature 1..21: note = Description of Artificial Sequence: Synthetic primer
  source 1..21: mol_type = other DNA
gctgagtgaactgcactgtga

SEQ ID NO: 87 - DNA, 20 bp, synthetic construct
  misc_feature 1..20: note = Description of Artificial Sequence: Synthetic primer
  source 1..20: mol_type = other DNA
gaattctttgccgaaatgga

SEQ ID NO: 88 - DNA, 20 bp, synthetic construct
  misc_feature 1..20: note = Description of Artificial Sequence: Synthetic primer
  source 1..20: mol_type = other DNA
tcactagcaagctctcaggc

SEQ ID NO: 89 - DNA, 19 bp, synthetic construct
  misc_feature 1..19: note = Description of Artificial Sequence: Synthetic primer
  source 1..19: mol_type = other DNA
aacaacgaggagtctgccc

SEQ ID NO: 90 - DNA, 24 bp, synthetic construct
  misc_feature 1..24: note = Description of Artificial Sequence: Synthetic primer
  source 1..24: mol_type = other DNA
gcagacagtgaccatctacagctt

SEQ ID NO: 91 - DNA, 20 bp, synthetic construct
  misc_feature 1..20: note = Description of Artificial Sequence: Synthetic primer
  source 1..20: mol_type = other DNA
caatccctctcgtccagtcg

SEQ ID NO: 92 - DNA, 20 bp, synthetic construct
  misc_feature 1..20: note = Description of Artificial Sequence: Synthetic primer
  source 1..20: mol_type = other DNA
tgcttggactatgggaggtc

SEQ ID NO: 93 - DNA, 20 bp, synthetic construct
  misc_feature 1..20: note = Description of Artificial Sequence: Synthetic primer
  source 1..20: mol_type = other DNA
gcaggtgcttcaaaaccatt

SEQ ID NO: 94 - DNA, 20 bp, synthetic construct
  misc_feature 1..20: note = Description of Artificial Sequence: Synthetic primer
  source 1..20: mol_type = other DNA
tcaggtggtcagcttctcct

SEQ ID NO: 95 - DNA, 20 bp, synthetic construct
  misc_feature 1..20: note = Description of Artificial Sequence: Synthetic primer
  source 1..20: mol_type = other DNA
aagcaaaccttctggctcaa

SEQ ID NO: 96 - DNA, 20 bp, synthetic construct
  misc_feature 1..20: note = Description of Artificial Sequence: Synthetic primer
  source 1..20: mol_type = other DNA
ccacacaggtgaaccctttt

SEQ ID NO: 97 - DNA, 20 bp, synthetic construct
  misc_feature 1..20: note = Description of Artificial Sequence: Synthetic primer
  source 1..20: mol_type = other DNA
ggacacatgctcacatacgg

SEQ ID NO: 98 - DNA, 19 bp, synthetic construct
  misc_feature 1..19: note = Description of Artificial Sequence: Synthetic primer
  source 1..19: mol_type = other DNA
attcgatccatgtgcctga

SEQ ID NO: 99 - DNA, 20 bp, synthetic construct
  misc_feature 1..20: note = Description of Artificial Sequence: Synthetic primer
  source 1..20: mol_type = other DNA
caatgctggaatttgtggaa

SEQ ID NO: 100 - DNA, 20 bp, synthetic construct
  misc_feature 1..20: note = Description of Artificial Sequence: Synthetic primer
  source 1..20: mol_type = other DNA
ggggtgattccctagagagg

SEQ ID NO: 101 - DNA, 20 bp, synthetic construct
  misc_feature 1..20: note = Description of Artificial Sequence: Synthetic primer
  source 1..20: mol_type = other DNA
aagcaggacagacaggcaag

SEQ ID NO: 102 - DNA, 20 bp, synthetic construct
  misc_feature 1..20: note = Description of Artificial Sequence: Synthetic primer
  source 1..20: mol_type = other DNA
gagggtcagcagtgatggat

SEQ ID NO: 103 - DNA, 20 bp, synthetic construct
  misc_feature 1..20: note = Description of Artificial Sequence: Synthetic primer
  source 1..20: mol_type = other DNA
tggaaaaggagaatgggaga

SEQ ID NO: 104 - DNA, 20 bp, synthetic construct
  misc_feature 1..20: note = Description of Artificial Sequence: Synthetic primer
  source 1..20: mol_type = other DNA
tggtcaagtttgccttgtca

SEQ ID NO: 105 - DNA, 21 bp, synthetic construct
  misc_feature 1..21: note = Description of Artificial Sequence: Synthetic primer
  source 1..21: mol_type = other DNA
ggaatgactgaatcggaacaa

SEQ ID NO: 106 - DNA, 20 bp, synthetic construct
  misc_feature 1..20: note = Description of Artificial Sequence: Synthetic primer
  source 1..20: mol_type = other DNA
cctccagcatcttccacatt

SEQ ID NO: 107 - DNA, 20 bp, synthetic construct
  misc_feature 1..20: note = Description of Artificial Sequence: Synthetic primer
  source 1..20: mol_type = other DNA
gaagcacccttcagcagttc

SEQ ID NO: 108 - DNA, 22 bp, synthetic construct
  misc_feature 1..22: note = Description of Artificial Sequence: Synthetic primer
  source 1..22: mol_type = other DNA
ccacagtttcagcgcagtaata

SEQ ID NO: 109 - DNA, 20 bp, synthetic construct
  misc_feature 1..20: note = Description of Artificial Sequence: Synthetic primer
  source 1..20: mol_type = other DNA
atcagccagcacacacactt

SEQ ID NO: 110 - DNA, 20 bp, synthetic construct
  misc_feature 1..20: note = Description of Artificial Sequence: Synthetic primer
  source 1..20: mol_type = other DNA
ccctgtcaggagggacagat

SEQ ID NO: 111 - DNA, 20 bp, synthetic construct
  misc_feature 1..20: note = Description of Artificial Sequence: Synthetic primer
  source 1..20: mol_type = other DNA
ggctcaccggaagcatgaat

SEQ ID NO: 112 - DNA, 20 bp, synthetic construct
  misc_feature 1..20: note = Description of Artificial Sequence: Synthetic primer
  source 1..20: mol_type = other DNA
aagctacaagcaggttcgct

SEQ ID NO: 113 - DNA, 20 bp, synthetic construct
  misc_feature 1..20: note = Description of Artificial Sequence: Synthetic primer
  source 1..20: mol_type = other DNA
aataacagggtccatcccgc

SEQ ID NO: 114 - DNA, 20 bp, synthetic construct
  misc_feature 1..20: note = Description of Artificial Sequence: Synthetic primer
  source 1..20: mol_type = other DNA
tgttccctccacctggaata

SEQ ID NO: 115 - DNA, 20 bp, synthetic construct
  misc_feature 1..20: note = Description of Artificial Sequence: Synthetic primer
  source 1..20: mol_type = other DNA
gggaaaatccaaagcaggat

SEQ ID NO: 116 - DNA, 20 bp, synthetic construct
  misc_feature 1..20: note = Description of Artificial Sequence: Synthetic primer
  source 1..20: mol_type = other DNA
tcctaggtccctcaaaagca

SEQ ID NO: 117 - DNA, 20 bp, synthetic construct
  misc_feature 1..20: note = Description of Artificial Sequence: Synthetic primer
  source 1..20: mol_type = other DNA
gtccccaacgctctaacaaa

SEQ ID NO: 118 - DNA, 20 bp, synthetic construct
  misc_feature 1..20: note = Description of Artificial Sequence: Synthetic primer
  source 1..20: mol_type = other DNA
gttagagcgttggggacctt

SEQ ID NO: 119 - DNA, 21 bp, synthetic construct
  misc_feature 1..21: note = Description of Artificial Sequence: Synthetic primer
  source 1..21: mol_type = other DNA
cacatgcagagaactgagctg

SEQ ID NO: 120 - DNA, 19 bp, synthetic construct
  misc_feature 1..19: note = Description of Artificial Sequence: Synthetic primer
  source 1..19: mol_type = other DNA
gttggggtaagcacgaagg

SEQ ID NO: 121 - DNA, 20 bp, synthetic construct
  misc_feature 1..20: note = Description of Artificial Sequence: Synthetic primer
  source 1..20: mol_type = other DNA
tttccaggagggtgactcag

SEQ ID NO: 122 - DNA, 20 bp, synthetic construct
  misc_feature 1..20: note = Description of Artificial Sequence: Synthetic primer
  source 1..20: mol_type = other DNA
ttctctgcatgtgacctccc

SEQ ID NO: 123 - DNA, 20 bp, synthetic construct
  misc_feature 1..20: note = Description of Artificial Sequence: Synthetic primer
  source 1..20: mol_type = other DNA
acacactcacagagggttgg

SEQ ID NO: 124 - DNA, 20 bp, synthetic construct
  misc_feature 1..20: note = Description of Artificial Sequence: Synthetic primer
  source 1..20: mol_type = other DNA
tgagtcaccctcctggaaac

SEQ ID NO: 125 - DNA, 20 bp, synthetic construct
  misc_feature 1..20: note = Description of Artificial Sequence: Synthetic primer
  source 1..20: mol_type = other DNA
ctccttccagagcacctcag

SEQ ID NO: 126 - DNA, 20 bp, synthetic construct
  misc_feature 1..20: note = Description of Artificial Sequence: Synthetic primer
  source 1..20: mol_type = other DNA
gctgggctcctccttgtact

SEQ ID NO: 127 - DNA, 20 bp, synthetic construct
  misc_feature 1..20: note = Description of Artificial Sequence: Synthetic primer
  source 1..20: mol_type = other DNA
gctgctgcccataaagtagc

SEQ ID NO: 128 - DNA, 19 bp, synthetic construct
  misc_feature 1..19: note = Description of Artificial Sequence: Synthetic primer
  source 1..19: mol_type = other DNA
ggactgtggcccaggtact

SEQ ID NO: 129 - DNA, 20 bp, synthetic construct
  misc_feature 1..20: note = Description of Artificial Sequence: Synthetic primer
  source 1..20: mol_type = other DNA
ggcctcataggacaggaggt

SEQ ID NO: 130 - DNA, 20 bp, synthetic construct
  misc_feature 1..20: note = Description of Artificial Sequence: Synthetic primer
  source 1..20: mol_type = other DNA
ttatgggcagcagctcagtt

SEQ ID NO: 131 - DNA, 20 bp, synthetic construct
  misc_feature 1..20: note = Description of Artificial Sequence: Synthetic primer
  source 1..20: mol_type = other DNA
gacattttcctggacgcttg

SEQ ID NO: 132 - DNA, 20 bp, synthetic construct
  misc_feature 1..20: note = Description of Artificial Sequence: Synthetic primer
  source 1..20: mol_type = other DNA
ccctccccatggctttaggt

SEQ ID NO: 133 - DNA, 20 bp, synthetic construct
  misc_feature 1..20: note = Description of Artificial Sequence: Synthetic primer
  source 1..20: mol_type = other DNA
agctccatgcgcttgacatt

SEQ ID NO: 134 - DNA, 20 bp, synthetic construct
  misc_feature 1..20: note = Description of Artificial Sequence: Synthetic primer
  source 1..20: mol_type = other DNA
agcgtccaggaaaatgtcaa

SEQ ID NO: 135 - DNA, 20 bp, synthetic construct
  misc_feature 1..20: note = Description of Artificial Sequence: Synthetic primer
  source 1..20: mol_type = other DNA
atgaccctcacactccaagg

SEQ ID NO: 136 - DNA, 20 bp, synthetic construct
  misc_feature 1..20: note = Description of Artificial Sequence: Synthetic primer
  source 1..20: mol_type = other DNA
gttgggtgctccagctttta

SEQ ID NO: 137 - DNA, 20 bp, synthetic construct
  misc_feature 1..20: note = Description of Artificial Sequence: Synthetic primer
  source 1..20: mol_type = other DNA
cctcaaaactcctggactcg

SEQ ID NO: 138 - protein, 1434 aa, synthetic construct
  REGION 1..1434: note = Description of Artificial Sequence: Synthetic polypeptide
MDYKDHDGDYKDHDIDYKDDDDKMAPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPIAGSKASPKKKRKVGRALINYPYDVPDYAS

SEQ ID NO: 139 - protein, 1483 aa, synthetic construct
  REGION 1..1483: note = Description of Artificial Sequence: Synthetic polypeptide
MDYKDHDGDYKDHDIDYKDDDDKMAPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPIAGSKASPKKKRKVGRADALDDFDLDMLGSDALDDFDLDMLGSDALDDFDLDMLGSDALDDFDLDMLINYPYDVPDYAS

SEQ ID NO: 140 - protein, 3844 aa, synthetic construct
  REGION 1..3844: note = Description of Artificial Sequence: Synthetic polypeptide
MDYKDHDGDYKDHDIDYKDDDDKMAPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPIAGSKASPKKKRKVGRAAENVVEPGPPSAKRPKLSSPALSASASDGTDFGSLFDLEHDLPDELINSTELGLTNGGDINQLQTSLGMVQDAASKHKQLSELLRSGSSPNLNMGVGGPGQVMASQAQQSSPGLGLINSMVKSPMTQAGLTSPNMGMGTSGPNQGPTQSTGMMNSPVNQPAMGMNTGMNAGMNPGMLAAGNGQGIMPNQVMNGSIGAGRGRQNMQYPNPGMGSAGNLLTEPLQQGSPQMGGQTGLRGPQPLKMGMMNNPNPYGSPYTQNPGQQIGASGLGLQIQTKTVLSNNLSPFAMDKKAVPGGGMPNMGQQPAPQVQQPGLVTPVAQGMGSGAHTADPEKRKLIQQQLVLLLHAHKCQRREQANGEVRQCNLPHCRTMKNVLNHMTHCQSGKSCQVAHCASSRQIISHWKNCTRHDCPVCLPLKNAGDKRNQQPILTGAPVGLGNPSSLGVGQQSAPNLSTVSQIDPSSIERAYAALGLPYQVNQMPTQPQVQAKNQQNQQPGQSPQGMRPMSNMSASPMGVNGGVGVQTPSLLSDSMLHSAINSQNPMMSENASVPSMGPMPTAAQPSTTGIRKQWHEDITQDLRNHLVHKLVQAIFPTPDPAALKDRRMENLVAYARKVEGDMYESANNRAEYYHLLAEKIYKIQKELEEKRRTRLQKQNMLPNAAGMVPVSMNPGPNMGQPQPGMTSNGPLPDPSMIRGSVPNQMMPRITPQSGLNQFGQMSMAQPPIVPRQTPPLQHHGQLAQPGALNPPMGYGPRMQQPSNQGQFLPQTQFPSQGMNVTNIPLAPSSGQAPVSQAQMSSSSCPVNSPIMPPGSQGSHIHCPQLPQPALHQNSPSPVPSRTPTPHHTPPSIGAQQPPATTIPAPVPTPPAMPPGPQSQALHPPPRQTPTPPTTQLPQQVQPSLPAAPSADQPQQQPRSQQSTAASVPTPTAPLLPPQPATPLSQPAVSIEGQVSNPPSTSSTEVNSQAIAEKQPSQEVKMEAKMEVDQPEPADTQPEDISESKVEDCKMESTETEERSTELKTEIKEEEDQPSTSATQSSPAPGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQAAGPVSQGKAAGQVTPPTPPQTAQPPLPGPPPAAVEMAMQIQRAAETQRQMAHVQIFQRPIQHQMPPMTPMAPMGMNPPPMTRGPSGHLEPGMGPTGMQQQPPWSQGGLPQPQQLQSGMPRPAMMSVAQHGQPLNMAPQPGLGQVGISPLKPGTVSQQALQNLLRTLRSPSSPLQQQQVLSILHANPQLLAAFIKQRAAKYANSNPQPIPGQPGMPQGQPGLQPPTMPGQQGVHSNPAMQNMNPMQAGVQRAGLPQQQPQQQLQPPMGGMSPQAQQMNMNHNTMPSQFRDILRRQQMMQQQQQQGAGPGIGPGMANHNQFQQPQGVGYPPQQQQRMQHHMQQMQQGNMGQIGQLPQALGAEAGASLQAYQQRLLQQQMGSPVQPNPMSPQQHMLPNQAQSPHLQGQQIPNSLSNQVRSPQPVPSPRPQSQPPHSSPSPRMQPQPSPHHVSPQTSSPHPGLVAAQANPMEQGHFASPDQNSMLSQLASNPGMANLHGASATDLGLSTDNSDLNSNLSQSTLDIHYPYDVPDYAS

SEQ ID NO: 141 - protein, 2048 aa, synthetic construct
  REGION 1..2048: note = Description of Artificial Sequence: Synthetic polypeptide
MDYKDHDGDYKDHDIDYKDDDDKMAPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPIAGSKASPKKKRKVGRAIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDYPYDVPDYAS

SEQ ID NO: 142 - protein, 2048 aa, synthetic construct
  REGION 1..2048: note = Description of Artificial Sequence: Synthetic polypeptide
MDYKDHDGDYKDHDIDYKDDDDKMAPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPIAGSKASPKKKRKVGRAIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLYSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDYPYDVPDYAS

SEQ ID NO: 143 - protein, 2048 aa, synthetic construct
  REGION 1..2048: note = Description of Artificial Sequence: Synthetic polypeptide
MDYKDHDGDYKDHDIDYKDDDDKMAPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPIAGSKASPKKKRKVGRAIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLEEAQWSTMCMLVELHTQSQDYPYDVPDYAS

SEQ ID NO: 144 - protein, 2048 aa, synthetic construct
  REGION 1..2048: note = Description of Artificial Sequence: Synthetic polypeptide
MDYKDHDGDYKDHDIDYKDDDDKMAPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPIAGSKASPKKKRKVGRAIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKRFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDYPYDVPDYAS

SEQ ID NO: 145 - protein, 2048 aa, synthetic construct
  REGION 1..2048: note = Description of Artificial Sequence: Synthetic polypeptide
MDYKDHDGDYKDHDIDYKDDDDKMAPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPIAGSKASPKKKRKVGRAIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWFKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDYPYDVPDYAS

SEQ ID NO: 146 - protein, 2048 aa, synthetic construct
  REGION 1..2048: note = Description of Artificial Sequence: Synthetic polypeptide
MDYKDHDGDYKDHDIDYKDDDDKMAPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPIAGSKASPKKKRKVGRAIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYIWWLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDYPYDVPDYAS

SEQ ID NO: 147 - protein, 2048 aa, synthetic construct
  REGION 1..2048: note = Description of Artificial Sequence: Synthetic polypeptide
MDYKDHDGDYKDHDIDYKDDDDKMAPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPIAGSKASPKKKRKVGRAIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYAEILIGYLAAVKKSGATTGAIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDYPYDVPDYAS

SEQ ID NO: 148 - protein, 1155 aa, synthetic construct
  REGION 1..1155: note = Description of Artificial Sequence: Synthetic polypeptide
MAAFKPNPINYILGLAIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIAAALPFSRTWDDSFNNKVLVLGSEAQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVRSRADPKKKRKVEASGSGRADALDDFDLDMLGSDALDDFDLDMLGSDALDDFDLDMLGSDALDDFDLDMLINSR

SEQ ID NO: 149 - protein, 1726 aa, synthetic construct
  REGION 1..1726: note = Description of Artificial Sequence: Synthetic polypeptide
MAAFKPNPINYILGLAIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIAAALPFSRTWDDSFNNKVLVLGSEAQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVRSRADPKKKRKVEASGRAIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDYPYDVPDYAS

SEQ ID NO: 150 - protein, 295 aa, synthetic construct
  REGION 1..295: note = Description of Artificial Sequence: Synthetic polypeptide
MDYKDHDGDYKDHDIDYKDDDDKMAPKKKRKVGRGMAQAALEPGEKPYACPECGKSFSDCRDLARHQRTHTGEKPYKCPECGKSFSRSDDLVRHQRTHTGEKPYKCPECGKSFSQSSNLVRHQRTHTGEKPYACPECGKSFSTSGELVRHQRTHTGEKPYKCPECGKSFSQRAHLERHQRTHTGEKPYKCPECGKSFSQAGHLASHQRTHTGKKTSGQAGQASPKKKRKVGRADALDDFDLDMLGSDALDDFDLDMLGSDALDDFDLDMLGSDALDDFDLDMLINYPYDVPDYAS

SEQ ID NO: 151 - protein, 860 aa, synthetic construct
  REGION 1..860: note = Description of Artificial Sequence: Synthetic polypeptide
MDYKDHDGDYKDHDIDYKDDDDKMAPKKKRKVGRGMAQAALEPGEKPYACPECGKSFSDCRDLARHQRTHTGEKPYKCPECGKSFSRSDDLVRHQRTHTGEKPYKCPECGKSFSQSSNLVRHQRTHTGEKPYACPECGKSFSTSGELVRHQRTHTGEKPYKCPECGKSFSQRAHLERHQRTHTGEKPYKCPECGKSFSQAGHLASHQRTHTGKKTSGQAGQASPKKKRKVGRAIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDYPYDVPDYAS

SEQ ID NO: 152 - DNA, 20 bp, synthetic construct
  misc_feature 1..20: note = Description of Artificial Sequence: Synthetic oligonucleotide
  source 1..20: mol_type = other DNA
cctggtcttcaatgagaaga

SEQ ID NO: 153 - DNA, 20 bp, synthetic construct
  misc_feature 1..20: note = Description of Artificial Sequence: Synthetic oligonucleotide
  source 1..20: mol_type = other DNA
gattaggacatgaacatggg

SEQ ID NO: 154 - DNA, 20 bp, synthetic construct
  misc_feature 1..20: note = Description of Artificial Sequence: Synthetic oligonucleotide
  source 1..20: mol_type = other DNA
cctcttctacattaacctta

SEQ ID NO: 155 - DNA, 20 bp, synthetic construct
  misc_feature 1..20: note = Description of Artificial Sequence: Synthetic oligonucleotide
  source 1..20: mol_type = other DNA
tttttgaagccagcaatcgt

SEQ ID NO: 156 - DNA, 20 bp, synthetic construct
  misc_feature 1..20: note = Description of Artificial Sequence: Synthetic oligonucleotide
  source 1..20: mol_type = other DNA
cgttagtttctggaggctct

SEQ ID NO: 157 - DNA, 20 bp, synthetic construct
  misc_feature 1..20: note = Description of Artificial Sequence: Synthetic oligonucleotide
  source 1..20: mol_type = other DNA
acaaattaccacgaatgtag

SEQ ID NO: 158 - DNA, 20 bp, synthetic construct
  misc_feature 1..20: note = Description of Artificial Sequence: Synthetic oligonucleotide
  source 1..20: mol_type = other DNA
tggcctgggcgcctgtctat

SEQ ID NO: 159 - DNA, 20 bp, synthetic construct
  misc_feature 1..20: note = Description of Artificial Sequence: Synthetic oligonucleotide
  source 1..20: mol_type = other DNA
attttgtaaataaggtcttc

SEQ ID NO: 160 - DNA, 20 bp, synthetic construct
  misc_feature 1..20: note = Description of Artificial Sequence: Synthetic oligonucleotide
  source 1..20: mol_type = other DNA
agcaacaggggatggggcag

SEQ ID NO: 161 - DNA, 20 bp, synthetic construct
  misc_feature 1..20: note = Description of Artificial Sequence: Synthetic oligonucleotide
  source 1..20: mol_type = other DNA
aggactcgtagtatgcaggc

SEQ ID NO: 162 - DNA, 20 bp, synthetic construct
  misc_feature 1..20: note = Description of Artificial Sequence: Synthetic oligonucleotide
  source 1..20: mol_type = other DNA
ctgagccaccaactatttaa

SEQ ID NO: 163 - DNA, 20 bp, synthetic construct
  misc_feature 1..20: note = Description of Artificial Sequence: Synthetic oligonucleotide
  source 1..20: mol_type = other DNA
ctgagccaccaactatttaa

SEQ ID NO: 164 - DNA, 20 bp, synthetic construct
  misc_feature 1..20: note = Description of Artificial Sequence: Synthetic oligonucleotide
  source 1..20: mol_type = other DNA
actctgggtcggttacggaa

SEQ ID NO: 165 - DNA, 20 bp, synthetic construct
  misc_feature 1..20: note = Description of Artificial Sequence: Synthetic oligonucleotide
  source 1..20: mol_type = other DNA
gggctgggcttagcttggga

SEQ ID NO: 166 - DNA, 20 bp, synthetic construct
  misc_feature 1..20: note = Description of Artificial Sequence: Synthetic oligonucleotide
  source 1..20: mol_type = other DNA
atagggaggggctctggagc

SEQ ID NO: 167 - DNA, 20 bp, synthetic construct
  misc_feature 1..20: note = Description of Artificial Sequence: Synthetic oligonucleotide
  source 1..20: mol_type = other DNA
atgggaaaagatacctgagt

SEQ ID NO: 168 - DNA, 20 bp, synthetic construct
  misc_feature 1..20: note = Description of Artificial Sequence: Synthetic oligonucleotide
  source 1..20: mol_type = other DNA
tgggagcgttgtgtcgcagc

SEQ ID NO: 169 - DNA, 20 bp, synthetic construct
  misc_feature 1..20: note = Description of Artificial Sequence: Synthetic oligonucleotide
  source 1..20: mol_type = other DNA
tggaaaggctttcattttct

SEQ ID NO: 170 - DNA, 20 bp, synthetic construct
  misc_feature 1..20: note = Description of Artificial Sequence: Synthetic oligonucleotide
  source 1..20: mol_type = other DNA
gtatctcgcagctccaatac

SEQ ID NO: 171 - DNA, 20 bp, synthetic construct
  misc_feature 1..20: note = Description of Artificial Sequence: Synthetic oligonucleotide
  source 1..20: mol_type = other DNA
acgcattcccctcggtttga

SEQ ID NO: 172 - DNA, 20 bp, synthetic construct
  misc_feature 1..20: note = Description of Artificial Sequence: Synthetic oligonucleotide
  source 1..20: mol_type = other DNA
tcggaagcttttcttctcag

SEQ ID NO: 173 - DNA, 20 bp, synthetic construct
  misc_feature 1..20: note = Description of Artificial Sequence: Synthetic oligonucleotide
  source 1..20: mol_type = other DNA
cgaaagggcgtgcgcgcccg

SEQ ID NO: 174 - DNA, 20 bp, synthetic construct
  misc_feature 1..20: note = Description of Artificial Sequence: Synthetic oligonucleotide
  source 1..20: mol_type = other DNA
ccggcgaaagggaagcggcc

SEQ ID NO: 175 - DNA, 20 bp, synthetic construct
  misc_feature 1..20: note = Description of Artificial Sequence: Synthetic oligonucleotide
  source 1..20: mol_type = other DNA
ggctgcgcacgcccatcccc

SEQ ID NO: 176 - DNA, 20 bp, synthetic construct
  misc_feature 1..20: note = Description of Artificial Sequence: Synthetic oligonucleotide
  source 1..20: mol_type = other DNA
ggggcttgcaggtggttcgc

SEQ ID NO: 177 - DNA, 20 bp, synthetic construct
  misc_feature 1..20: note = Description of Artificial Sequence: Synthetic oligonucleotide
  source 1..20: mol_type = other DNA
cgagctaaagagcggatgcc

SEQ ID NO: 178 - DNA, 20 bp, synthetic construct
  misc_feature 1..20: note = Description of Artificial Sequence: Synthetic oligonucleotide
  source 1..20: mol_type = other DNA
agagggcgggagcagggcca

SEQ ID NO: 179 - DNA, 20 bp, synthetic construct
  misc_feature 1..20: note = Description of Artificial Sequence: Synthetic oligonucleotide
  source 1..20: mol_type = other DNA
aaccggctcttaactctttg

SEQ ID NO: 180 - DNA, 20 bp, synthetic construct
  misc_feature 1..20: note = Description of Artificial Sequence: Synthetic oligonucleotide
  source 1..20: mol_type = other DNA
caggagcggcgagcggggtc

SEQ ID NO: 181 - DNA, 20 bp, synthetic construct
  misc_feature 1..20: note = Description of Artificial Sequence: Synthetic oligonucleotide
  source 1..20: mol_type = other DNA
gggtatcagatggcaaagtt

SEQ ID NO: 182 - DNA, 20 bp, synthetic construct
  misc_feature 1..20: note = Description of Artificial Sequence: Synthetic oligonucleotide
  source 1..20: mol_type = other DNA
tcataggctgccggcgattg

SEQ ID NO: 183 - DNA, 20 bp, synthetic construct
  misc_feature 1..20: note = Description of Artificial Sequence: Synthetic oligonucleotide
  source 1..20: mol_type = other DNA
gaggttggccaggagcagcg

SEQ ID NO: 184 - DNA, 20 bp, synthetic construct
  misc_feature 1..20: note = Description of Artificial Sequence: Synthetic oligonucleotide
  source 1..20: mol_type = other DNA
aattagccccgcacggcgag

SEQ ID NO: 185 - DNA, 20 bp, synthetic construct
  misc_feature 1..20: note = Description of Artificial Sequence: Synthetic oligonucleotide
  source 1..20: mol_type = other DNA
tcccctgggtaggagtacag

SEQ ID NO: 186 - DNA, 20 bp, synthetic construct
  misc_feature 1..20: note = Description of Artificial Sequence: Synthetic oligonucleotide
  source 1..20: mol_type = other DNA
ggttgttagctgcggtcagc

SEQ ID NO: 187 - DNA, 20 bp, synthetic construct
  misc_feature 1..20: note = Description of Artificial Sequence: Synthetic oligonucleotide
  source 1..20: mol_type = other DNA
ggtggagaacagggggcgcc

SEQ ID NO: 188 - DNA, 23 bp, synthetic construct
  misc_feature 1..23: note = Description of Artificial Sequence: Synthetic oligonucleotide
  source 1..23: mol_type = other DNA
  variation 21: note = any nucleotide residue A, G, C, or T
cctggtcttcaatgagaagangg

SEQ ID NO: 189 - DNA, 23 bp, synthetic construct
  misc_feature 1..23: note = Description of Artificial Sequence: Synthetic oligonucleotide
  variation 21: note = any nucleotide residue A, G, C, or T
  source 1..23: mol_type = other DNA
gattaggacatgaacatgggngg

SEQ ID NO: 190 - DNA, 23 bp, synthetic construct
  misc_feature 1..23: note = Description of Artificial Sequence: Synthetic oligonucleotide
  variation 21: note = any nucleotide residue A, G, C, or T
  source 1..23: mol_type = other DNA
cctcttctacattaaccttangg

SEQ ID NO: 191 - DNA, 23 bp, synthetic construct
  misc_feature 1..23: note = Description of Artificial Sequence: Synthetic oligonucleotide
  variation 21: note = any nucleotide residue A, G, C, or T
  source 1..23: mol_type = other DNA
tttttgaagccagcaatcgtngg

SEQ ID NO: 192 - DNA, 23 bp, synthetic construct
  misc_feature 1..23: note = Description of Artificial Sequence: Synthetic oligonucleotide
  variation 21: note = any nucleotide residue A, G, C, or T
  source 1..23: mol_type = other DNA
cgttagtttctggaggctctngg

SEQ ID NO: 193 - DNA, 23 bp, synthetic construct
  misc_feature 1..23: note = Description of Artificial Sequence: Synthetic oligonucleotide
  variation 21: note = any nucleotide residue A, G, C, or T
  source 1..23: mol_type = other DNA
acaaattaccacgaatgtagngg

SEQ ID NO: 194 - DNA, 23 bp, synthetic construct
  misc_feature 1..23: note = Description of Artificial Sequence: Synthetic oligonucleotide
  variation 21: note = any nucleotide residue A, G, C, or T
  source 1..23: mol_type = other DNA
tggcctgggcgcctgtctatngg

SEQ ID NO: 195 - DNA, 23 bp, synthetic construct
  misc_feature 1..23: note = Description of Artificial Sequence: Synthetic oligonucleotide
  variation 21: note = any nucleotide residue A, G, C, or T
  source 1..23: mol_type = other DNA
attttgtaaataaggtcttcngg

SEQ ID NO: 196 - DNA, 23 bp, synthetic construct
  misc_feature 1..23: note = Description of Artificial Sequence: Synthetic oligonucleotide
  variation 21: note = any nucleotide residue A, G, C, or T
  source 1..23: mol_type = other DNA
agcaacaggggatggggcagngg

SEQ ID NO: 197 - DNA, 23 bp, synthetic construct
  misc_feature 1..23: note = Description of Artificial Sequence: Synthetic oligonucleotide
  variation 21: note = any nucleotide residue A, G, C, or T
  source 1..23: mol_type = other DNA
aggactcgtagtatgcaggcngg

SEQ ID NO: 198 - DNA, 23 bp, synthetic construct
  misc_feature 1..23: note = Description of Artificial Sequence: Synthetic oligonucleotide
  variation 21: note = any nucleotide residue A, G, C, or T
  source 1..23: mol_type = other DNA
ctgagccaccaactatttaangg

SEQ ID NO: 199 - DNA, 23 bp, synthetic construct
  misc_feature 1..23: note = Description of Artificial Sequence: Synthetic oligonucleotide
  variation 21: note = any nucleotide residue A, G, C, or T
  source 1..23: mol_type = other DNA
ctgagccaccaactatttaangg

SEQ ID NO: 200 - DNA, 23 bp, synthetic construct
  misc_feature 1..23: note = Description of Artificial Sequence: Synthetic oligonucleotide
  variation 21: note = any nucleotide residue A, G, C, or T
  source 1..23: mol_type = other DNA
actctgggtcggttacggaangg

SEQ ID NO: 201 - DNA, 23 bp, synthetic construct
  misc_feature 1..23: note = Description of Artificial Sequence: Synthetic oligonucleotide
  variation 21: note = any nucleotide residue A, G, C, or T
  source 1..23: mol_type = other DNA
gggctgggcttagcttgggangg

SEQ ID NO: 202 - DNA, 23 bp, synthetic construct
  misc_feature 1..23: note = Description of Artificial Sequence: Synthetic oligonucleotide
  variation 21: note = any nucleotide residue A, G, C, or T
  source 1..23: mol_type = other DNA
atagggaggggctctggagcngg

SEQ ID NO: 203 - DNA, 23 bp, synthetic construct
  misc_feature 1..23: note = Description of Artificial Sequence: Synthetic oligonucleotide
  variation 21: note = any nucleotide residue A, G, C, or T
  source 1..23: mol_type = other DNA
atgggaaaagatacctgagtngg

SEQ ID NO: 204 - DNA, 23 bp, synthetic construct
  misc_feature 1..23: note = Description of Artificial Sequence: Synthetic oligonucleotide
  variation 21: note = any nucleotide residue A, G, C, or T
  source 1..23: mol_type = other DNA
tgggagcgttgtgtcgcagcngg

SEQ ID NO: 205 - DNA, 23 bp, synthetic construct
  misc_feature 1..23: note = Description of Artificial Sequence: Synthetic oligonucleotide
  variation 21: note = any nucleotide residue A, G, C, or T
  source 1..23: mol_type = other DNA
tggaaaggctttcattttctngg

SEQ ID NO: 206 - DNA, 23 bp, synthetic construct
  misc_feature 1..23: note = Description of Artificial Sequence: Synthetic oligonucleotide
  variation 21: note = any nucleotide residue A, G, C, or T
  source 1..23: mol_type = other DNA
gtatctcgcagctccaatacngg

SEQ ID NO: 207 - DNA, 23 bp, synthetic construct
  misc_feature 1..23: note = Description of Artificial Sequence: Synthetic oligonucleotide
  variation 21: note = any nucleotide residue A, G, C, or T
  source 1..23: mol_type = other DNA
acgcattcccctcggtttgangg

SEQ ID NO: 208 - DNA, 23 bp, synthetic construct
  misc_feature 1..23: note = Description of Artificial Sequence: Synthetic oligonucleotide
  variation 21: note = any nucleotide residue A, G, C, or T
  source 1..23: mol_type = other DNA
tcggaagcttttcttctcagngg

SEQ ID NO: 209 - DNA, 23 bp, synthetic construct
  misc_feature 1..23: note = Description of Artificial Sequence: Synthetic oligonucleotide
  variation 21: note = any nucleotide residue A, G, C, or T
  source 1..23: mol_type = other DNA
cgaaagggcgtgcgcgcccgngg

SEQ ID NO: 210 - DNA, 23 bp, synthetic construct
  misc_feature 1..23: note = Description of Artificial Sequence: Synthetic oligonucleotide
  variation 21: note = any nucleotide residue A, G, C, or T
  source 1..23: mol_type = other DNA
ccggcgaaagggaagcggccngg

SEQ ID NO: 211 - DNA, 23 bp, synthetic construct
  misc_feature 1..23: note = Description of Artificial Sequence: Synthetic oligonucleotide
  variation 21: note = any nucleotide residue A, G, C, or T
  source 1..23: mol_type = other DNA
ggctgcgcacgcccatccccngg

SEQ ID NO: 212 - DNA, 23 bp, synthetic construct
  misc_feature 1..23: note = Description of Artificial Sequence: Synthetic oligonucleotide
  variation 21: note = any nucleotide residue A, G, C, or T
  source 1..23: mol_type = other DNA
ggggcttgcaggtggttcgcngg

SEQ ID NO: 213 - DNA, 23 bp, synthetic construct
  misc_feature 1..23: note = Description of Artificial Sequence: Synthetic oligonucleotide
  variation 21: note = any nucleotide residue A, G, C, or T
  source 1..23: mol_type = other DNA
cgagctaaagagcggatgccngg

SEQ ID NO: 214 - DNA, 23 bp, synthetic construct
  misc_feature 1..23: note = Description of Artificial Sequence: Synthetic oligonucleotide
  variation 21: note = any nucleotide residue A, G, C, or T
  source 1..23: mol_type = other DNA
agagggcgggagcagggccangg

SEQ ID NO: 215 - DNA, 23 bp, synthetic construct
  misc_feature 1..23: note = Description of Artificial Sequence: Synthetic oligonucleotide
  variation 21: note = any nucleotide residue A, G, C, or T
  source 1..23: mol_type = other DNA
aaccggctcttaactctttgngg

SEQ ID NO: 216 - DNA, 23 bp, synthetic construct
  misc_feature 1..23: note = Description of Artificial Sequence: Synthetic oligonucleotide
  variation 21: note = any nucleotide residue A, G, C, or T
  source 1..23: mol_type = other DNA
caggagcggcgagcggggtcngg

SEQ ID NO: 217 - DNA, 23 bp, synthetic construct
  misc_feature 1..23: note = Description of Artificial Sequence: Synthetic oligonucleotide
  variation 21: note = any nucleotide residue A, G, C, or T
  source 1..23: mol_type = other DNA
gggtatcagatggcaaagttngg

SEQ ID NO: 218 - DNA, 23 bp, synthetic construct
  misc_feature 1..23: note = Description of Artificial Sequence: Synthetic oligonucleotide
  variation 21: note = any nucleotide residue A, G, C, or T
  source 1..23: mol_type = other DNA
tcataggctgccggcgattgngg

SEQ ID NO: 219 - DNA, 23 bp, synthetic construct
  misc_feature 1..23: note = Description of Artificial Sequence: Synthetic oligonucleotide
  variation 21: note = any nucleotide residue A, G, C, or T
  source 1..23: mol_type = other DNA
gaggttggccaggagcagcgngg

SEQ ID NO: 220 - DNA, 23 bp, synthetic construct
  misc_feature 1..23: note = Description of Artificial Sequence: Synthetic oligonucleotide
  variation 21: note = any nucleotide residue A, G, C, or T
  source 1..23: mol_type = other DNA
aattagccccgcacggcgagngg

SEQ ID NO: 221 - DNA, 23 bp, synthetic construct
  misc_feature 1..23: note = Description of Artificial Sequence: Synthetic oligonucleotide
  variation 21: note = any nucleotide residue A, G, C, or T
  source 1..23: mol_type = other DNA
tcccctgggtaggagtacagngg

SEQ ID NO: 222 - DNA, 23 bp, synthetic construct
  misc_feature 1..23: note = Description of Artificial Sequence: Synthetic oligonucleotide
  variation 21: note = any nucleotide residue A, G, C, or T
  source 1..23: mol_type = other DNA
ggttgttagctgcggtcagcngg

SEQ ID NO: 223 - DNA, 23 bp, synthetic construct
  misc_feature 1..23: note = Description of Artificial Sequence: Synthetic oligonucleotide
  variation 21: note = any nucleotide residue A, G, C, or T
  source 1..23: mol_type = other DNA
ggtggagaacagggggcgccngg

SEQ ID NO: 224 - DNA, 20 bp, synthetic construct
  misc_feature 1..20: note = Description of Artificial Sequence: Synthetic oligonucleotide
  source 1..20: mol_type = other DNA
gggggcgcgagtgatcagct

SEQ ID NO: 225 - DNA, 20 bp, synthetic construct
  misc_feature 1..20: note = Description of Artificial Sequence: Synthetic oligonucleotide
  source 1..20: mol_type = other DNA
cccgggtctcctaggggacg

SEQ ID NO: 226 - DNA, 20 bp, synthetic construct
  misc_feature 1..20: note = Description of Artificial Sequence: Synthetic oligonucleotide
  source 1..20: mol_type = other DNA
tggtccggagaaagaaggcg

SEQ ID NO: 227 - DNA, 20 bp, synthetic construct
  misc_feature 1..20: note = Description of Artificial Sequence: Synthetic oligonucleotide
  source 1..20: mol_type = other DNA
gtctccgggctcggaaactt

SEQ ID NO: 228 - DNA, 20 bp, synthetic construct
  misc_feature 1..20: note = Description of Artificial Sequence: Synthetic oligonucleotide
  source 1..20: mol_type = other DNA
agcgccagagcgcgagagcg

SEQ ID NO: 229 - DNA, 20 bp, synthetic construct
  misc_feature 1..20: note = Description of Artificial Sequence: Synthetic oligonucleotide
  source 1..20: mol_type = other DNA
cgattccggccgcgttcccc

SEQ ID NO: 230 - DNA, 20 bp, synthetic construct
  misc_feature 1..20: note = Description of Artificial Sequence: Synthetic oligonucleotide
  source 1..20: mol_type = other DNA
gttgtgcgggctgatgcgcc

SEQ ID NO: 231 - DNA, 25 bp, synthetic construct
  misc_feature 1..25: note = Description of Artificial Sequence: Synthetic oligonucleotide
  source 1..25: mol_type = other DNA
cctcgtgtgttcctgggcctgctgc

SEQ ID NO: 232 - DNA, 25 bp, synthetic construct
  misc_feature 1..25: note = Description of Artificial Sequence: Synthetic oligonucleotide
  source 1..25: mol_type = other DNA
tcccataaacaggattctgctcaga

SEQ ID NO: 233 - DNA, 25 bp, synthetic construct
  misc_feature 1..25: note = Description of Artificial Sequence: Synthetic oligonucleotide
  source 1..25: mol_type = other DNA
caccggccagatgacagaacagaaa

SEQ ID NO: 234 - DNA, 27 bp, synthetic construct
  misc_feature 1..27: note = Description of Artificial Sequence: Synthetic oligonucleotide
  source 1..27: mol_type = other DNA
ttgtttgaaaatgccatttgtagggct

SEQ ID NO: 235 - DNA, 25 bp, synthetic construct
  misc_feature 1..25: note = Description of Artificial Sequence: Synthetic oligonucleotide
  source 1..25: mol_type = other DNA
aaacgcagcaggcccaggaacacac

SEQ ID NO: 236 - DNA, 25 bp, synthetic construct
  misc_feature 1..25: note = Description of Artificial Sequence: Synthetic oligonucleotide
  source 1..25: mol_type = other DNA
aaactctgagcagaatcctgtttat

SEQ ID NO: 237 - DNA, 25 bp, synthetic construct
  misc_feature 1..25: note = Description of Artificial Sequence: Synthetic oligonucleotide
  source 1..25: mol_type = other DNA
aaactttctgttctgtcatctggcc

SEQ ID NO: 238 - DNA, 27 bp, synthetic construct
  misc_feature 1..27: note = Description of Artificial Sequence: Synthetic oligonucleotide
  source 1..27: mol_type = other DNA
aaacagccctacaaatggcattttcaa

SEQ ID NO: 239 - DNA, 25 bp, synthetic construct
  misc_feature 1..25: note = Description of Artificial Sequence: Synthetic oligonucleotide
  source 1..25: mol_type = other DNA
cctcgtctgcttctgccgaacctca

SEQ ID NO: 240 - DNA, 25 bp, synthetic construct
  misc_feature 1..25: note = Description of Artificial Sequence: Synthetic oligonucleotide
  source 1..25: mol_type = other DNA
tcccacctaaagagcttgtaggccg

SEQ ID NO: 241 - DNA, 25 bp, synthetic construct
  misc_feature 1..25: note = Description of Artificial Sequence: Synthetic oligonucleotide
  source 1..25: mol_type = other DNA
caccgagagctggctacccgtccct

SEQ ID NO: 242 - DNA, 27 bp, synthetic construct
  misc_feature 1..27: note = Description of Artificial Sequence: Synthetic oligonucleotide
  source 1..27: mol_type = other DNA
ttgtttgcggtccttgtttatcagtag

SEQ ID NO: 243 - DNA, 25 bp, synthetic construct
  misc_feature 1..25: note = Description of Artificial Sequence: Synthetic oligonucleotide
  source 1..25: mol_type = other DNA
aaactgaggttcggcagaagcagac

SEQ ID NO: 244 - DNA, 25 bp, synthetic construct
  misc_feature 1..25: note = Description of Artificial Sequence: Synthetic oligonucleotide
  source 1..25: mol_type = other DNA
aaaccggcctacaagctctttaggt

SEQ ID NO: 245 - DNA, 25 bp, synthetic construct
  misc_feature 1..25: note = Description of Artificial Sequence: Synthetic oligonucleotide
  source 1..25: mol_type = other DNA
aaacagggacgggtagccagctctc

SEQ ID NO: 246 - DNA, 27 bp, synthetic construct
  misc_feature 1..27: note = Description of Artificial Sequence: Synthetic oligonucleotide
  source 1..27: mol_type = other DNA
aaacctactgataaacaaggaccgcaa

SEQ ID NO: 247 - DNA, 25 bp, synthetic construct
  misc_feature 1..25: note = Description of Artificial Sequence: Synthetic oligonucleotide
  source 1..25: mol_type = other DNA
cctcggagctggctacccgtcccta

SEQ ID NO: 248 - DNA, 25 bp, synthetic construct
  misc_feature 1..25: note = Description of Artificial Sequence: Synthetic oligonucleotide
  source 1..25: mol_type = other DNA
tcccactttggctgggtttaaacca

SEQ ID NO: 249 - DNA, 25 bp, synthetic construct
  misc_feature 1..25: note = Description of Artificial Sequence: Synthetic oligonucleotide
  source 1..25: mol_type = other DNA
caccggtcagctcagggttttggta

SEQ ID NO: 250 - DNA, 27 bp, synthetic construct
  misc_feature 1..27: note = Description of Artificial Sequence: Synthetic oligonucleotide
  source 1..27: mol_type = other DNA
ttgtttggagttagctccccgacccag

SEQ ID NO: 251 - DNA, 25 bp, synthetic construct
  misc_feature 1..25: note = Description of Artificial Sequence: Synthetic oligonucleotide
  source 1..25: mol_type = other DNA
aaactagggacgggtagccagctcc

SEQ ID NO: 252 - DNA, 25 bp, synthetic construct
  misc_feature 1..25: note = Description of Artificial Sequence: Synthetic oligonucleotide
  source 1..25: mol_type = other DNA
aaactggtttaaacccagccaaagt

SEQ ID NO: 253 - DNA, 25 bp, synthetic construct
  misc_feature 1..25: note = Description of Artificial Sequence: Synthetic oligonucleotide
  source 1..25: mol_type = other DNA
aaactaccaaaaccctgagctgacc

SEQ ID NO: 254 - DNA, 27 bp, synthetic construct
  misc_feature 1..27: note = Description of Artificial Sequence: Synthetic oligonucleotide
  source 1..27: mol_type = other DNA
aaacctgggtcggggagctaactccaa

SEQ ID NO: 255 - DNA, 20 bp, synthetic construct
  misc_feature 1..20: note = Description of Artificial Sequence: Synthetic oligonucleotide
  source 1..20: mol_type = other DNA
tgtgttcctgggcctgctgc

SEQ ID NO: 256 - DNA, 20 bp, synthetic construct
  misc_feature 1..20: note = Description of Artificial Sequence: Synthetic oligonucleotide
  source 1..20: mol_type = other DNA
taaacaggattctgctcaga

SEQ ID NO: 257 - DNA, 20 bp, synthetic construct
  misc_feature 1..20: note = Description of Artificial Sequence: Synthetic oligonucleotide
  source 1..20: mol_type = other DNA
gccagatgacagaacagaaa

SEQ ID NO: 258 - DNA, 20 bp, synthetic construct
  misc_feature 1..20: note = Description of Artificial Sequence: Synthetic oligonucleotide
  source 1..20: mol_type = other DNA
aaaatgccatttgtagggct

SEQ ID NO: 259 - DNA, 20 bp, synthetic construct
  misc_feature 1..20: note = Description of Artificial Sequence: Synthetic oligonucleotide
  source 1..20: mol_type = other DNA
gcagcaggcccaggaacaca

SEQ ID NO: 260 - DNA, 20 bp, synthetic construct
  misc_feature 1..20: note = Description of Artificial Sequence: Synthetic oligonucleotide
  source 1..20: mol_type = other DNA
tctgagcagaatcctgttta

SEQ ID NO: 261 - DNA, 20 bp, synthetic construct
  misc_feature 1..20: note = Description of Artificial Sequence: Synthetic oligonucleotide
  source 1..20: mol_type = other DNA
tttctgttctgtcatctggc

SEQ ID NO: 262 - DNA, 20 bp, synthetic construct
  misc_feature 1..20: note = Description of Artificial Sequence: Synthetic oligonucleotide
  source 1..20: mol_type = other DNA
agccctacaaatggcatttt

SEQ ID NO: 263 - DNA, 20 bp, synthetic construct
  misc_feature 1..20: note = Description of Artificial Sequence: Synthetic oligonucleotide
  source 1..20: mol_type = other DNA
tctgcttctgccgaacctca

SEQ ID NO: 264 - DNA, 20 bp, synthetic construct
  misc_feature 1..20: note = Description of Artificial Sequence: Synthetic oligonucleotide
  source 1..20: mol_type = other DNA
cctaaagagcttgtaggccg

SEQ ID NO: 265 - DNA, 20 bp, synthetic construct
  misc_feature 1..20: note = Description of Artificial Sequence: Synthetic oligonucleotide
  source 1..20: mol_type = other DNA
agagctggctacccgtccct

SEQ ID NO: 266 - DNA, 20 bp, synthetic construct
  misc_feature 1..20: note = Description of Artificial Sequence: Synthetic oligonucleotide
  source 1..20: mol_type = other DNA
cggtccttgtttatcagtag

SEQ ID NO: 267 - DNA, 20 bp, synthetic construct
  misc_feature 1..20: note = Description of Artificial Sequence: Synthetic oligonucleotide
  source 1..20: mol_type = other DNA
tgaggttcggcagaagcaga

SEQ ID NO: 268 - DNA, 20 bp, synthetic construct
  misc_feature 1..20: note = Description of Artificial Sequence: Synthetic oligonucleotide
  source 1..20: mol_type = other DNA
cggcctacaagctctttagg

SEQ ID NO: 269 - DNA, 20 bp, synthetic construct
  misc_feature 1..20: note = Description of Artificial Sequence: Synthetic oligonucleotide
  source 1..20: mol_type = other DNA
agggacgggtagccagctct

SEQ ID NO: 270 - DNA, 20 bp, synthetic construct
  misc_feature 1..20: note = Description of Artificial Sequence: Synthetic oligonucleotide
  source 1..20: mol_type = other DNA
ctactgataaacaaggaccg

SEQ ID NO: 271 - DNA, 20 bp, synthetic construct
  misc_feature 1..20: note = Description of Artificial Sequence: Synthetic oligonucleotide
  source 1..20: mol_type = other DNA
gagctggctacccgtcccta

SEQ ID NO: 272 - DNA, 20 bp, synthetic construct
  misc_feature 1..20: note = Description of Artificial Sequence: Synthetic oligonucleotide
  source 1..20: mol_type = other DNA
ctttggctgggtttaaacca

SEQ ID NO: 273 - DNA, 20 bp, synthetic construct
  misc_feature 1..20: note = Description of Artificial Sequence: Synthetic oligonucleotide
  source 1..20: mol_type = other DNA
gtcagctcagggttttggta

SEQ ID NO: 274 - DNA, 20 bp, synthetic construct
  misc_feature 1..20: note = Description of Artificial Sequence: Synthetic oligonucleotide
  source 1..20: mol_type = other DNA
gagttagctccccgacccag

SEQ ID NO: 275 - DNA, 20 bp, synthetic construct
  misc_feature 1..20: note = Description of Artificial Sequence: Synthetic oligonucleotide
  source 1..20: mol_type = other DNA
tagggacgggtagccagctc

SEQ ID NO: 276 - DNA, 20 bp, synthetic construct
  misc_feature 1..20: note = Description of Artificial Sequence: Synthetic oligonucleotide
  source 1..20: mol_type = other DNA
tggtttaaacccagccaaag

SEQ ID NO: 277 - DNA, 20 bp, synthetic construct
  misc_feature 1..20: note = Description of Artificial Sequence: Synthetic oligonucleotide
  source 1..20: mol_type = other DNA
taccaaaaccctgagctgac

SEQ ID NO: 278 - DNA, 20 bp, synthetic construct
  misc_feature 1..20: note = Description of Artificial Sequence: Synthetic oligonucleotide
  source 1..20: mol_type = other DNA
ctgggtcggggagctaactc